(19) United States
(12) Patent Application Publication
(10) Pub. No.: US 2018/0104664 A1
(43) Pub. Date: Apr. 19, 2018

(54) TEXTURED SURFACES FOR POLYNUCLEOTIDE SYNTHESIS

(71) Applicant: Twist Bioscience Corporation, San Francisco, CA (US)

(72) Inventors: Andres FERNANDEZ, San Francisco, CA (US); Pierre INDERMUHLE, Berkeley, CA (US); Eugene P. MARSH, El Granada, CA (US); William BANYAI, San Francisco, CA (US); Bill James PECK, Santa Clara, CA (US)

(21) Appl. No.: 15/835,342

(22) Filed: Dec. 7, 2017

Related U.S. Application Data
(63) Continuation of application No. PCT/US17/45105, filed on Aug. 2, 2017.
(60) Provisional application No. 62/370,548, filed on Aug. 3, 2016.

(51) Int. Cl. B01J 19/00; C12N 15/10

(52) U.S. Cl. CPC B01J 19/0046; C12N 15/1093; B01J 2219/00596; B01J 2219/00722; B01J 2219/00587; B01J 2219/00497

(57) ABSTRACT

Methods, devices and systems are provided herein for surfaces for de novo polynucleotide synthesis that provide for increased polynucleotide yield. Surfaces described herein comprise a texture that increases surface area provide for increased polynucleotide yield compared to non-textured surfaces. In addition, the patterned placement of nucleoside coupling reagent spanning such surfaces provides for improved synthesis yield, representation, and a reduction in contamination on the surface between different polynucleotide species.

### CROSS-REFERENCE

This application claims the benefit of U.S. Provisional Application No. 62/370,548, filed Aug. 3, 2016, which application is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Highly efficient chemical gene synthesis with high fidelity and low cost has a central role in biotechnology and medicine, and in basic biomedical research. De novo gene synthesis is a powerful tool for basic biological research and biotechnology applications. While various methods are known for the synthesis of relatively short fragments in a small scale, these techniques suffer from scalability, automation, speed, accuracy, and cost. There is a need for devices for simple, reproducible, scalable, less error-prone and cost-effective methods that guarantee successful synthesis of desired genes and are amenable to automation.

### BRIEF SUMMARY

Provided herein is a device for polynucleotide synthesis, the device comprising: a solid support comprising a surface; a plurality of loci on the surface, wherein each of the loci comprises: an inner region, wherein the inner region comprises a plurality of recesses or protrusions; and an outer region that comprises a plurality of first molecules, wherein the outer region spans and extends beyond the inner region, and wherein each of the first molecules binds to the surface and comprises a reactive group capable of binding to a nucleoside. Further provided herein is a device wherein the plurality of loci are arranged in clusters. Further provided herein is a device wherein each cluster comprises 50 to 500 loci. Further provided herein is a wherein each cluster comprises about 121 loci. Further provided herein is a device wherein the outer region has a diameter of up to 100 um. Further provided herein is a device wherein the outer region has a diameter of about 60 um. Further provided herein is a wherein the inner region has a diameter of about 55 um. Further provided herein is a device wherein the inner region has a diameter 80% to 95% shorter than the diameter of the outer region. Further provided herein is a device wherein the inner region has a diameter 2 um to 20 um shorter than the diameter of the outer region. Further provided herein is a device wherein the inner region has a diameter about 5 um shorter than the diameter of the outer region. Further provided herein is a device wherein each of the recesses or protrusions have an etch depth of 100 um to 1000 nm. Further provided herein is a device wherein each of the recesses or protrusions has an etch depth of 200 um to 500 nm. Further provided herein is a device wherein each of the recesses or protrusions has a width of 100 to 500 um. Further provided herein is a device wherein each of the recesses or protrusions has a width of 300 to 330 um. Further provided herein is a device wherein each of the recesses or protrusions has a pitch length of about 2 to 3 times a width of the recesses or protrusions. Further provided herein is a device wherein each of the recesses or protrusions has a depth of about 60% to 125% of a pitch length. Further provided herein is a wherein each of the recesses or protrusions has a patch of up to 1 um. Further provided herein is device a wherein the solid support has a tensile strength of 1 MPa to 300 MPa. Further provided herein is a device wherein the solid support has a tensile strength of 1 MPa to 10 MPa. Further provided herein is a device wherein the solid support has a stiffness of 1 GPa to 500 GPa. Further provided herein is a device wherein the solid support has a stiffness of 1 GPa to 10 GPa. Further provided herein is a device wherein the solid support comprises nylon, nitrocellulose, or polypropylene. Further provided herein is a device wherein the solid support comprises silicon, silicon dioxide, silicon nitride, polytetrafluoroethylene, polypropylene, polystyrene, polycarbonate, gold, or platinum. Further provided herein is a device wherein each of the first molecules is a silane. Further provided herein is a device wherein the silane is an aminosilane. Further provided herein is a device wherein each of the first molecules is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Further provided herein is a device comprising a plurality of second molecules, wherein plurality of second molecules is located on the surface in a region surrounding the outer region of each of the loci, and wherein each second molecule binds to the surface and lacks a reactive group capable of binding to the nucleoside. Further provided herein is a device wherein the second molecule is a fluorosilane. Further provided herein is a device wherein the fluorosilane is (tridecafluoro-1,1,2,2-tetrahydrooctyl)trichlorosilane, perfluorooctyltrichlorosilane, perfluorooctyltriethoxysilane, or perfluorooctyltrimethoxychloro silane.

Provided herein is a method for polynucleotide synthesis, comprising: providing predetermined sequences for polynucleotides; providing the device of any one of claims 1 to 27; and synthesizing the polynucleotides. Further provided herein is a method wherein the polynucleotides comprise at least 30,000 non-identical polynucleotides. Further provided herein is a method wherein the at least 30,000 non-identical polynucleotides encode for at least 750 genes. Further provided herein is a method wherein the at least 30,000 non-identical polynucleotides have an aggregate error rate of less than 1 in 1000 bases compared to the predetermined sequences for polynucleotides. Further provided herein is a method wherein the at least 30,000 non-identical polynucleotides have an aggregate error rate of less than 1 in 1500 bases compared to the predetermined sequences for the polynucleotides. Further provided herein is a method wherein at least 80% of at least 30,000 non-identical polynucleotides have no errors compared to the predetermined sequences for the polynucleotides. Further provided herein is a method wherein at least 89% of at least 30,000 non-identical polynucleotides have no errors compared to the predetermined sequences for the polynucleotides.

Provided herein is a method for gene synthesis, comprising: providing predetermined sequences for polynucleotides; providing the device of any one of claims 1 to 27; synthesizing the polynucleotides; and assembling the polynucleotides to form a plurality of genes. Further provided herein is a method further comprising releasing the polynucleotides prior to step (d).

Provided herein is a system for polynucleotide synthesis, the system comprising: a material deposition device comprising plurality of reagents for polynucleotide synthesis and a plurality of nozzles for depositing the plurality of reagents for polynucleotide synthesis; a computer for controlling the release of the plurality of reagents for polynucleotide synthesis from the plurality of nozzles; and the device of any one of claims 1 to 27 for synthesis of polynucleotides.

### BRIEF DESCRIPTION OF THE DRAWINGS

FIG. 1A depicts a 16×16 array of clusters of loci.

FIG. 1B depicts an arrangement of clusters of loci.

FIG. 1C depicts an exemplary arrangement of loci within a cluster of loci.

FIG. 2A depicts a cross-section view of an exemplary textured locus comprising an array of raised texture features.

FIG. 2B depicts a cross-section view of an exemplary textured locus comprising an array of recessed texture features.

FIG. 3A depicts a top view of an exemplary array of textured loci.

FIG. 3B depicts a top view an exemplary textured locus.

FIG. 4A depicts an exemplary device with one side polished.

FIG. 4B depicts a process of lithographic printing on an exemplary device.

FIG. 4C recessed features of an exemplary device formed by the lithographic printing.

FIG. 4D illustrates a process of oxidizing an exemplary device.

FIG. 4E illustrates a process of lithographic printing an exemplary device using a photosensitive lack.

FIG. 4F illustrates a process of oxide etching an exemplary device to form a textured silicon surface with fiducial structures.

FIG. 5 illustrates an exemplary device having a patterned surface comprising loci coated with a molecule for coupling to a nucleoside, wherein the loci are surrounding by regions coated with an agent that does not couple a nucleoside.

FIGS. 6A-6F illustrates an exemplary method for generating a surface having a reduction in nucleoside-coupling agent density.

FIG. 6A illustrates a process of cleaning an exemplary device with an oxygen plasma.

FIG. 6B illustrates a process of coated an exemplary device with a photosensitive lack.

FIG. 6C illustrates a process of optically lithographing an exemplary device.

FIG. 6D illustrates a process of depositing a first molecule on the exposed surfaces of an exemplary device.

FIG. 6E illustrates a process of stripping away the photosensitive lack.

FIG. 6F illustrates a process of binding a third molecule to the surface of an exemplary device.

FIG. 7A illustrates a region of a surface of an exemplary device for polynucleotide synthesis that is coated with a silane that binds the surface and couples nucleoside.

FIG. 7B illustrates a region of a surface of an exemplary device for polynucleotide synthesis coated with a mixture of silanes, one silane that binds the surface and couples polynucleotide, and another silane that binds the surface and does not couple to nucleoside.

FIGS. 8A-8G illustrates a method a method for generating a surface having a reduction in nucleoside-coupling agent density.

FIG. 8A illustrates a process of cleaning an exemplary device with an oxygen plasma.

FIG. 8B illustrates a process of coating an exemplary device with a first chemical layer that binds the surface of the device and binds nucleoside.

FIG. 8C illustrates a process of coating an exemplary device with a photosensitive lack.

FIG. 8D illustrates a process of optically lithographing an exemplary device.

FIG. 8E illustrates a process patterning of the first chemical layer.

FIG. 8F illustrates a process of depositing a second chemical layer, comprising a molecule that binds the surface and does not bind nucleoside on the surface of an exemplary device, per an embodiment of the disclosure herein

FIG. 8G illustrates a process of stripping away the photosensitive lack.

FIG. 9 is a diagram demonstrating an exemplary process workflow from oligonucleic synthesis to gene shipment.

FIG. 10 illustrates an outline of an exemplary system for nucleic acid synthesis, including an polynucleotide synthesizer, a device (wafer), schematics outlining the alignment of the system elements in multiple directions, and exemplary setups for reagent flow.

FIG. 11 illustrates an exemplary phosphoramidite chemistry for oligonucleotide synthesis.

FIG. 12 illustrates an exemplary device having fiducial markings.

FIG. 13 illustrates another exemplary device having fiducial markings.

FIG. 14 illustrates an exemplary computer system.

FIG. 15 is a block diagram illustrating a first architecture of an exemplary computer system.

FIG. 16 is a diagram demonstrating an exemplary network configured to incorporate a plurality of computer systems, a plurality of cell phones and personal data assistants, and Network Attached Storage (NAS).

FIG. 17 is a block diagram of an exemplary multiprocessor computer system using a shared virtual address memory space.

FIG. 18 is an image of an exemplary textured microfluidic device.

FIG. 19 is another image of an exemplary textured microfluidic device.

FIG. 20 is a close-up image of an exemplary textured microfluidic device.

FIG. 21 is a side-view of a slice of an exemplary textured microfluidic device.

FIG. 22A is a scanning electron micrograph of an exemplary textured microfluidic device.

FIG. 22B is a scanning electron micrograph of an exemplary textured microfluidic device.

FIG. 23A is a low magnification image of an exemplary cluster of non-textured loci.

FIG. 23B is a low magnification image of an exemplary cluster of textured loci.

FIG. 24A is a high magnification image of an exemplary cluster of non-textured loci.

FIG. 24B is a high magnification image of an exemplary cluster of textured loci.

FIG. 25A illustrates an exemplary arrangement of outer-textured loci.

FIG. 25B illustrates an exemplary outer-textured locus.

FIG. 26A illustrates a cross-section view of an exemplary outer-textured locus comprising arrangement of an array of raised texture features.

FIG. 26B illustrates a cross-section view of an exemplary outer-textured locus comprising an arrangement of an array of recessed texture features.

FIG. 27A is a low magnification image of an exemplary cluster of outer-textured loci.

FIG. 27B is a high magnification image of an exemplary cluster of outer-textured loci.

FIG. 28 illustrates an exemplary textured microfluidic device with a pattern of clusters of textured loci.

FIGS. 29A-29D are images of droplets dispensed onto an exemplary textured microfluidic device patterns of clusters of textured loci consistent with the arrangement of FIG. 28.

FIG. 29A depicts an image of 200 nL droplets dispensed onto an exemplary textured microfluidic device.

FIG. 29B depicts an image of 275 nL droplets dispensed onto an exemplary textured microfluidic device.

FIG. 29C depicts an image of 350 nL droplets dispensed onto an exemplary textured microfluidic device.

FIG. 29D depicts an image of 425 nL droplets dispensed onto an exemplary textured microfluidic device.

FIG. 30A is a chart depicting the dropout rates for a first exemplary device comprising textured loci, outer-textured loci and non-textured loci.

FIG. 30B is a chart depicting the dropout rates for a second exemplary device comprising textured loci, outer-textured loci and non-textured loci.

FIG. 30C is a chart depicting the dropout rates for a third exemplary device comprising textured loci, outer-textured loci and non-textured loci.

FIG. 30D is a chart depicting the dropout rates for a fourth exemplary device comprising textured loci, outer-textured loci and non-textured loci.

FIG. 31A illustrates an exemplary functionalized surface.

FIG. 31B displays BioAnalyzer data at five locations on the exemplary functionalized surface.

FIG. 32 displays BioAnalyzer data of surface extracted 100-mer oligonucleotides synthesized on an exemplary silicon oligonucleotide synthesis device.

FIG. 33 represents an exemplary sequence alignment, where “×” denotes a single base deletion, “star” denotes single base mutation, and “+” denotes low quality spots in Sanger sequencing.

FIG. 34 represents an exemplary sequence alignment, where “×” denotes a single base deletion, “star” denotes single base mutation, and “+” denotes low quality spots in Sanger sequencing.

FIG. 35 is an exemplary histogram for oligonucleotides encoding for 240 genes, with the length of oligonucleotide as the x-axis and number of oligonucleotide as the y-axis.

FIG. 36 is an exemplary histogram for polynucleotides collectively encoding for a gene, with the length of oligonucleotide as the x-axis and number of oligonucleotide as the y-axis.

FIG. 37A illustrates exemplary plots for DNA thickness per device for polynucleotides of 30, 50, and 80-mers when synthesized a surface.

FIG. 37B and DNA mass per device for polynucleotides of 30, 50, and 80-mers when synthesized a surface.

FIG. 38 illustrates the deletion rate at a given index of synthesized oligonucleotides for various silane solutions.

FIG. 39 illustrates average deletion and insertion rates of various textured microfluidic devices at a depth of 500 um.

FIG. 40 illustrates measured and expected yield enhancements of various exemplary textured microfluidic devices.

FIG. 41 illustrates deletion rates of four nucleic acid bases on exemplary textured microfluidic devices with different etch depths.

FIG. 42 illustrates relative deletion rates by texture type of various exemplary textured microfluidic devices with different etch depths.

FIG. 43 illustrates relative deletion rates by texture depth of various exemplary textured microfluidic devices with different etch depths.

FIG. 44 illustrates insertion rates by base of four nucleic acid bases on exemplary textured microfluidic devices with different etch depths.

FIG. 45 illustrates relative insertion rates by base texture type of exemplary textured microfluidic devices when compared to an untextured design.

### DETAILED DESCRIPTION

The present disclosure provides systems, methods, devices for rapid parallel synthesis of polynucleotide libraries with low error rates. The oligonucleotide synthesis steps described herein are “de novo,” meaning that oligonucleotides are built one monomer at a time to form a polymer. During de novo synthesis of polynucleotides, the crowding of single stranded polynucleotides extending from a surface results in an increase in error rates. To reduce the frequency of crowding-related errors, methods are provided herein to reduce the density of nucleoside-coupling agent bound to specific regions of the surface. At the same time, to compensate for the reduced density of polynucleotides extending from a surface, methods are disclosed herein to increase surface area so as to increase the yield of synthesized polynucleotides.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

Provided herein are methods and compositions for production of synthetic (i.e. de novo synthesized or chemically synthesizes) polynucleotides. The term oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude exons. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA.

Provided herein are methods and compositions for production of synthetic (i.e. de novo synthesized) genes. Libraries comprising synthetic genes may be constructed by a variety of methods described in further detail elsewhere herein, such as PCA, non-PCA gene assembly methods or hierarchical gene assembly, combining (“stitching”) two or more double-stranded polynucleotides to produce larger DNA units (i.e., a chassis). Libraries of large constructs may involve polynucleotides that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs can be bounded by an independently selected upper limit of about 5000, 10000, 20000 or 50000 base pairs. The synthesis of any number of polypeptide-segment encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein, may comprise at least one region encoding for exon sequence(s) without an intervening intron sequence found in the corresponding genomic sequence. Alternatively, the corresponding genomic sequence to a cDNA may lack intron sequence in the first place.

Unless otherwise stated, water contact angles mentioned herein correspond to measurements that would be taken on uncurved, smooth, or planar equivalents of the surfaces in question.

### Clusters and Loci

Provided herein is a device comprising a surface, wherein the surface is modified to support polynucleotide synthesis at predetermined locations and with a resulting low error rate, a low dropout rate, a high yield, and a high oligo representation. In some embodiments, the surface comprises a plurality of loci, wherein each locus comprises a plurality of first molecules deposited on the locus, wherein the first molecule binds to the surface and comprises a reactive group capable of binding to a nucleoside.

The terms “locus” and “loci,” as used herein, refer to a single discrete active region, and to a plurality of discrete active regions on the surface of the device, respectively, wherein the plurality of first molecules are deposited on said locus, and wherein the first molecule binds to the surface and comprises a reactive group capable of binding to a nucleoside. In some embodiments, the plurality of first molecule comprises one or a mixture of molecule(s), which binds to the surface and comprises a reactive group capable of binding to a nucleoside.

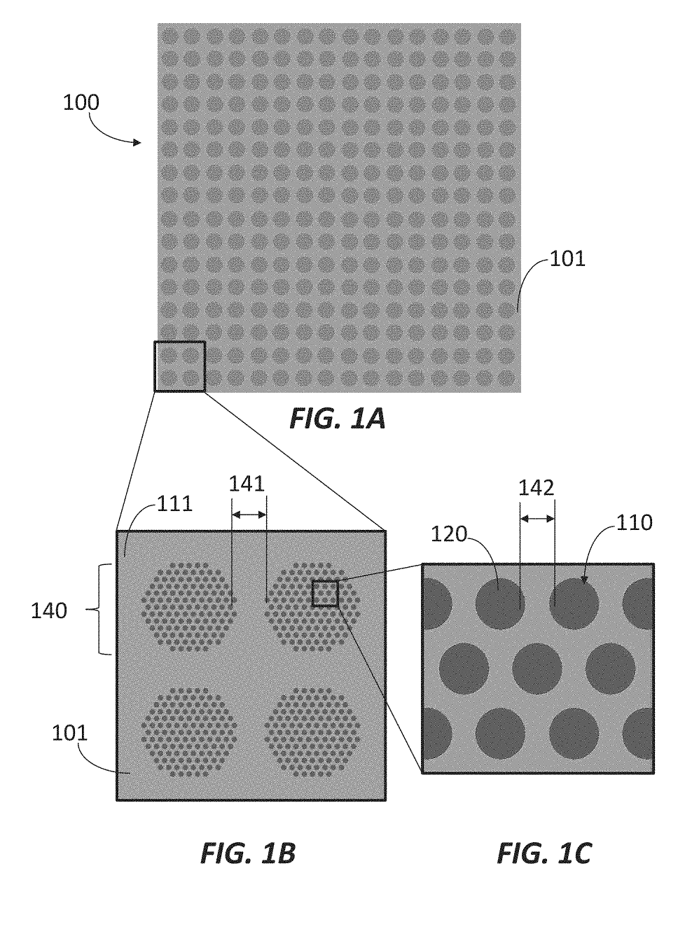

Referring to FIGS. 1A to 1C, an exemplary device 100 provided herein comprises a surface 101, wherein the surface 101 comprises a plurality of loci 110, wherein each locus 110 comprises a plurality of first molecules 120, wherein the plurality of first molecules 120 comprise a high-energy molecule, and wherein the first molecule binds to the surface 101 and comprises a reactive group capable of binding to a nucleoside, to synthesize a single sequence polynucleotide. In this arrangement, the plurality of the first molecules 120 deposited on each locus 110 exhibit a higher surface energy than the surface 101 of the device, and wherein the variation in the surface energy facilitates localization of droplets of a fluid onto the loci 110. In some embodiments, localization of droplets onto the loci 110 is altered by adjusting the pattern and geometry of the loci 110. In some instances, the high-energy molecules 120 on one locus 110 are capable of binding to the surface and comprise a reactive group capable of binding to a certain nucleoside to support the synthesis of a certain population of polynucleotides having a certain sequence, wherein the first molecules on another locus 110 are capable of binding to the surface and comprise a reactive group capable of binding to a different nucleoside to support the synthesis of a different population of polynucleotides having a different sequence.

In some instances, the surface 101 of the device 100 comprises a plurality of loci 110, wherein the plurality of loci 110 are arranged into a plurality of clusters 140, wherein each cluster 140 comprises a plurality of loci 110. Referring to FIGS. 1A to 1C, the surface 101 of the device 100 comprises a rectilinear array of 16 columns and 16 rows of clusters 140, wherein each cluster 140 comprises a hexagonal array of 156 loci 110, wherein each column and each row of clusters 140 are separated by a cluster gap 141. In some embodiments the centers of each of the plurality of loci 110 are positioned in a honeycomb lattice within the cluster 140, wherein each of the loci 110 are separated by a loci gap 142.

The shape of the loci described herein may comprises a circle, a triangle, a square, a rectangle, a hexagon, a polygon, an amorphous shape, a pixelated amorphous shape, or any shape that is known in the art, or any shapes that may be made by methods known in the art. The loci may be shaped to allow liquid to easily flow through without creating air bubbles.

The resolved loci may have a monodisperse size distribution, wherein two or more of the loci have approximately the same width, height, and/or length. In some embodiments, a loci has a limited number of shapes and/or sizes, for example, the resolved loci may be represented in 2, 3, 4, 5, 6, 7, 8, 9, 10, 12, 15, 20, or more distinct shapes, each having a monodisperse size. In some embodiments, a shape of a locus is repeated in multiple monodisperse size distributions, for example, 2, 3, 4, 5, 6, 7, 8, 9, 10, 12, 15, 20, or more monodisperse size distributions.

A monodisperse distribution of loci may be reflected in a unimodular distribution with a standard deviation of less than 25%, 20%, 15%, 10%, 5%, 3%, 2%, 1%, 0.1%, 0.05%, 0.01%, 0.001% of the mode or smaller. A monodisperse distribution of loci may be reflected in a unimodular distribution with a standard deviation of about 0.001% to about 25%. A monodisperse distribution of loci may be reflected in a unimodular distribution with a standard deviation of at least about 0.001%. A monodisperse distribution of loci may be reflected in a unimodular distribution with a standard deviation of at most about 25%. A monodisperse distribution of loci may be reflected in a unimodular distribution with a standard deviation of about 0.001% to about 5%. A monodisperse distribution of loci may be reflected in a unimodular distribution with a standard deviation of about 0.001%, about 0.01%, about 0.05%, about 0.1%, about 1%, about 2%, about 3%, about 5%, about 10%, about 15%, about 20%, or about 25%.

In some instances, the polynucleotides extending from a plurality of loci 110 within a cluster 140 collectively encode for a longer nucleic acid sequence, e.g., a gene. In some cases, a library of polynucleotides are synthesized on a plurality of loci 110, followed by the assembly of the polynucleotides into a large nucleic acid, such as gene.

In further instances, each locus 110 is surrounded by a passive region comprising a second molecule, wherein the second molecule comprises a low-energy molecule, wherein the second molecule binds to the surface and lacks a reactive group capable of coupling a nucleoside. The high-energy molecules in the active region exhibit a high surface energy. The low-energy molecules in the passive region exhibit a lower surface energy than the surface energy of the active region. Each locus 110 is surrounded by one or more passive regions, wherein the one or more passive region(s) comprise a plurality of low-energy molecules 111, and wherein the low-energy molecules 111 are capable of binding with the surface 101 but not with a nucleoside.

### Arrangements of Clusters and Loci

Provided herein is a device comprising a surface, wherein the surface is modified to support polynucleotide synthesis at predetermined locations and with a resulting low error rate, a low dropout rate, a high yield, and a high oligo representation. The surface may comprise a plurality of loci, wherein each locus comprises an active region of the surface, wherein each active region comprises a first molecule comprising a high-energy molecule, and wherein the first molecule binds to the surface and comprises a reactive group capable of binding to a nucleoside. The high-energy molecules in the active region exhibit a high surface energy.

Each locus may be surrounded by a passive region, wherein each passive region comprises a low-energy molecule, and wherein the low energy molecule is capable of coupling to the surface, but does not couple a nucleoside. In some embodiments, the low-energy molecules in the passive region exhibit a lower surface energy than the high-energy molecules in the active region.

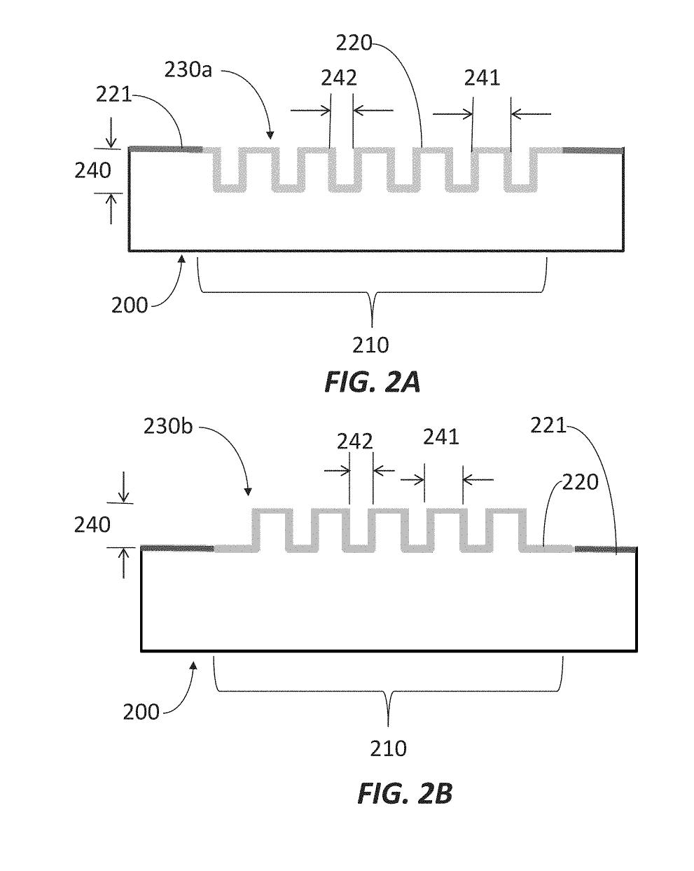

Referring to FIGS. 2A to 2C, the loci 210 are arranged within a cluster 240 in a triangular pattern, a rectilinear pattern, a pentagonal pattern, a honeycomb pattern, an octagonal pattern, polygonal pattern, an irregular array, or any combination thereof. In some embodiments, the shape of the cluster 240 comprises a circle, a triangle, a square, a rectangle, a hexagon, a polygon, an amorphous shape, a pixelated amorphous shape, or a closed zigzag shape. The clusters 240 are arranged in a triangular pattern, a rectilinear pattern, a pentagonal pattern, a honeycomb pattern, an octagonal pattern, polygonal pattern, an irregular array, or any combination thereof. Exemplary ranges for the density of loci is about 1 locus per mm² to about 1,000 loci per mm². In some instances, the number of loci on the surface of the device is about 5,000; 10,000; about 1,000,000, or more.

Provided herein are devices wherein a surface disclosed herein comprises a plurality of clusters, wherein the number of loci in a cluster is about 2 to about 500. The number of loci in a cluster may be about 10 to about 50, 50 to 500, 50 to 1000, or more than 1000. In exemplary arrangements, each cluster includes 109, 121, 130, or 137 loci. In some arrangements, the ratio between the diameter of a locus and the gap distance (pitch) between two loci is about 1:50 to about 50:1. In some arrangements, the ratio between the diameter of a locus and the gap distance between two loci is at least about 1:10. In some arrangements, the ratio between the diameter of a locus and the gap distance between two loci is at most about 10:1. In some embodiments, the ratio between the diameter of a locus and the gap distance between two loci is at about 1:50, about 1:20, about 1:10, about 1:5, about 1:2, about 1:1, about 2:1, about 5:1, about 10:1, about 20:1 or about 50:1.

Loci described herein may have width or diameter of about 5 μm to about 1,000 μm. In some arrangements, a locus has a width or diameter of at most about 1,000 μm. In some arrangements, a locus has a width or diameter of about 5 μm to about 10 μm, about 5 μm to about 50 μm, about 5 μm to about 100 μm, about 5 μm to about 200 μm, about 5 μm to about 300 μm, about 5 μm to about 400 μm, about 5 μm to about 500 μm, about 5 μm to about 600 μm, about 5 μm to about 700 μm, about 5 μm to about 800 μm, about 5 μm to about 1,000 μm, about 10 μm to about 50 μm, about 10 μm to about 100 μm, about 10 μm to about 200 μm, about 10 μm to about 300 μm, about 10 μm to about 400 μm, about 10 μm to about 500 μm, about 10 μm to about 600 μm, about 10 μm to about 700 μm, about 10 μm to about 800 μm, about 10 μm to about 1,000 μm, about 50 μm to about 100 μm, about 50 μm to about 200 μm, about 50 μm to about 300 μm, about 50 μm to about 400 μm, about 50 μm to about 500 μm, about 50 μm to about 600 μm, about 50 μm to about 700 μm, about 50 μm to about 800 μm, about 50 μm to about 1,000 μm, about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 1,000 μm, about 400 μm to about 500 μm, about 400 μm to about 600 μm, about 400 μm to about 700 μm, about 400 μm to about 800 μm, about 400 μm to about 1,000 μm, about 500 μm to about 600 μm, about 500 μm to about 700 μm, about 500 μm to about 800 μm, about 500 μm to about 1,000 μm, about 600 μm to about 700 μm, about 600 μm to about 800 μm, about 600 μm to about 1,000 μm, about 700 μm to about 800 μm, about 700 μm to about 1,000 μm, or about 800 μm to about 1,000 μm. In some embodiments, a locus has a width or diameter of about 5 μm, about 10 μm, about 50 μm, about 100 μm, about 200 μm, about 300 μm, about 400 μm, about 500 μm, about 600 μm, about 700 μm, about 800 μm, or about 1,000 μm. In some instances, loci within a cluster described herein are separated by a gap distance of about 1 μm to about 500 μm.

In some instance, the density of clusters within a region of a surface described herein is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more.

In some embodiments, a cluster has a width or diameter of about 0.05 mm to about 50 mm. In some embodiments, a cluster has a width or diameter of at least about 0.05 mm. In some embodiments, a cluster has a width or diameter of at most about 50 mm. In some embodiments, a cluster has a width or diameter of about 0.05 mm to about 0.1 mm, about 0.05 mm to about 0.25 mm, about 0.05 mm to about 0.5 mm, about 0.05 mm to about 1 mm, about 0.05 mm to about 2 mm, about 0.05 mm to about 5 mm, about 0.05 mm to about 10 mm, about 0.05 mm to about 20 mm, about 0.05 mm to about 50 mm, about 0.1 mm to about 0.25 mm, about 0.1 mm to about 0.5 mm, about 0.1 mm to about 1 mm, about 0.1 mm to about 2 mm, about 0.1 mm to about 5 mm, about 0.1 mm to about 10 mm, about 0.1 mm to about 20 mm, about 0.1 mm to about 50 mm, about 0.25 mm to about 0.5 mm, about 0.25 mm to about 1 mm, about 0.25 mm to about 2 mm, about 0.25 mm to about 5 mm, about 0.25 mm to about 10 mm, about 0.25 mm to about 20 mm, about 0.25 mm to about 50 mm, about 0.5 mm to about 1 mm, about 0.5 mm to about 2 mm, about 0.5 mm to about 5 mm, about 0.5 mm to about 10 mm, about 0.5 mm to about 20 mm, about 0.5 mm to about 50 mm, about 1 mm to about 2 mm, about 1 mm to about 5 mm, about 1 mm to about 10 mm, about 1 mm to about 20 mm, about 1 mm to about 50 mm, about 2 mm to about 5 mm, about 2 mm to about 10 mm, about 2 mm to about 20 mm, about 2 mm to about 50 mm, about 5 mm to about 10 mm, about 5 mm to about 20 mm, about 5 mm to about 50 mm, about 10 mm to about 20 mm, about 10 mm to about 50 mm, or about 20 mm to about 50 mm. In some embodiments, a cluster has a width or diameter of about 0.05 mm, about 0.1 mm, about 0.25 mm, about 0.5 mm, about 1 mm, about 2 mm, about 5 mm, about 10 mm, about 20 mm, or about 50 mm. Provided herein are surfaces which comprise at least 10, 100, 500, 1000, 2000, 3000, 4000, 5000, 6000, 7000, 8000, 9000, 10000, 11000, 12000, 13000, 14000, 15000, 20000, 30000, 40000, 50000 or more clusters,

In some instances, the clusters are arranged on the surface of the device in a triangular pattern, a circular pattern, a rectilinear pattern, a pentagonal pattern, a honeycomb pattern, an octagonal pattern, polygonal pattern, an irregular array, or any combination thereof.

In some instances, the width of a cluster is about 1 μm to about 10,000 μm. Exemplary cluster widths include, about 10 μm to about 25 μm, about 10 μm to about 50 μm, about 10 μm to about 75 μm, about 10 μm to about 100 μm, about 10 μm to about 250 μm, about 10 μm to about 500 μm, about 10 μm to about 750 μm, about 10 μm to about 1,000 μm, about 10 μm to about 2,500 μm, about 10 μm to about 5,000 μm, and about 10 μm to about 10,000 μm.

### Surfaces Comprising Texture Features

Provided herein are devices comprising a surface, wherein the surface is modified to support polynucleotide synthesis at predetermined locations and with a resulting low error rate, a low dropout rate, a high yield, and a high oligo representation. In some embodiments, the surface comprises a plurality of loci, wherein each locus comprises an active region of the surface, wherein each active region comprises a first molecule comprising a high-energy molecule, wherein first molecule the binds to the surface and comprises a reactive group capable of binding to a nucleoside. In some embodiments, the portion of the surface that is covered with the coupling agent comprises a locus, wherein a plurality of loci comprises a cluster, and wherein a plurality of clusters comprises a device. In some embodiments, a locus forms a hydrophilic region with a certain water contact angle.

In some instances, a portion of a surface of a device described herein is not covered with a high-energy molecule that comprises a reactive group capable of binding to a nucleoside reduces the yield of synthesized polynucleotides per area of the surface. As insufficient polynucleotide yields may impair subsequent downstream molecular biology processes, which utilize the synthesized polynucleotides (e.g., as gene assembly), the surface of the devices provided herein comprises a plurality of at least one of a recess and a protrusion, to increase the polynucleotide extension surface area.

In some instance, a surface of a device provided herein comprises a surface comprising a plurality of resolved loci, onto which nucleic acids or other molecules are deposited for polynucleotide synthesis, wherein the surface is smooth and substantially planar, or comprises a texture of raised and/or lowered features (e.g., three-dimensional features). In some instances, the surface of the device provided herein comprises plurality of recesses or protrusions that increase the surface area of the device, and polynucleotide yield. In some embodiments, plurality of recesses or protrusions are arranged in a pattern correspond to the locations of the loci. In some instances, a plurality of recesses or protrusions comprises an array of wells, microwells, channels, posts, or other raised or lowered features. In some instances, the plurality of recesses or protrusions is accessible to reagent deposition via a deposition device such as a polynucleotide synthesizer. In some cases, reagents and/or fluids may collect in a larger well in fluid communication one or of the plurality of recesses or protrusions. In some instances, the plurality of recesses or protrusions comprises a plurality of channels corresponding to a plurality of loci within a cluster, wherein the plurality of channels or wells are in fluid communication with one well of the cluster.

In a first exemplary arrangement, devices described herein comprise loci for polynucleotide extension comprising recesses or protrusions, wherein the loci extends beyond a boundary of the recesses or protrusions. In some embodiments, the surface of the device 200 comprises a plurality of loci 210, wherein each locus 210 comprises a recess 230a, or a protrusion 230b, per FIGS. 2A and 2B, respectively, and an active region 220. In some embodiments, the active region 220 of each locus 210 is surrounded by a passive region 221.

In some embodiments, each recess 230a or protrusion 230b has a depth 240 and a width 241, wherein the recess 230a or the protrusions 230b are separated by a pitch 242. A recess 230a or a protrusion 230b may have the same or different depth 240, width 241, volume, or any combination thereof, as another recess 230b or protrusion 230b within the same locus 210. The recess 230a or protrusions 230b of a locus 210 may have the same or different depth 240, width 241, volume, or any combination thereof, as the recess 230a or the protrusions 230b of another locus 210. The recess 230a or protrusions 230b of the loci 210 within a cluster of loci 210 may have the same or different depth 240, width 241, volume, or any combination thereof, as the recess 230a or the protrusions 230b of the loci 210 within another cluster of loci 210.

The recess 230b and the protrusion 230b may be designed to allow for controlled flow, and to ensure even mass transfer paths for polynucleotide synthesis on a surface of the device 200. The recess 230b and the protrusion 230b, and the methods of employing the device 200 thereby, may be designed for a specific or a range of chemical exposure times, for wash efficacy during polynucleotide synthesis, and/or for increased sweep efficiency. In some cases, sweep efficiency may be increased by ensuring that growing polynucleotide do not take up more than 50, 45, 40, 25, 20, 25, 20, 15, 14, 13, 12, 11, 10, 9, 8, 7, 6, 5, 4, 2, 2, 1%, of the initially available volume within the protrusion or recess 230a 230b. In some instances, standard silicon wafer processes may be employed to form devices 200 comprising protrusions or recesses 230a 230b with a high surface area and sufficient flow channels to allow for rapid chemical exchange and exposure with the surface of the device 200. The pitch 242 between recesses 230b or protrusions 230b may be designed to provide sufficient space for two times the length of the expected polynucleotide.

In some instances, the distance of the pitch is sufficient to accommodate a polynucleotide with a length of about 10 mer to about 2000 mer. In some instances, the distance of the pitch is sufficient to accommodate a polynucleotide with a length of about 10 mer to about 500 mer. In some instances, the distance of the pitch is sufficient to accommodate a polynucleotide with a length of at least about 10 mer.

In an exemplary arrangement, the pitch 242 is at least twice the length of the polynucleotide synthesized on the surface disclosed herein. The recess 230b and the protrusion 230b may be designed such that the depth 240 is sufficient for efficient washing. In one example, the depth 240 of the recess 230b or protrusion 230b is about half to about one and a half times the pitch 242 between recesses 230b or protrusions 230b, and the pitch 242 is about twice the width 241.

The surface area per device, represented herein as “S, ” is measured by the following equation, wherein d=depth; w=width; and pitch:

$$S = {p}^{2} ( 1 + \frac{π d}{2 p} ) . \qquad Equation 1$$

When a ratio of the pitch to the width is about 2, the following equation may be used to calculate the surface area of a device comprising a textured surface, wherein S₀ is the surface area of the untextured surface of the device:

$$S = {S}_{0} ( 1 + \frac{π d}{2 p} ) . \qquad Equation 2$$

In some arrangements the recess and the protrusion have a cross sectional shape comprising a circle, a triangle, a square, a pentagon, a hexagon, an octagon, a polygon, an irregular shape, or any combination thereof. In some embodiments, the recess and the protrusion are tapered inward or outward towards the surface of the device. The recess and the protrusion may have sharp, tapered, chamfered, or rounded edges. In some instances, perimeters of groups of recess and the protrusion may be marked by a different type of structural feature or by differential functionalization. In some cases, the recess or the protrusion varies along its height and depends on the composition of the device. For example, a device comprising a surface composed of a material other than silicon may have a different height than a silicon structure of the same width. The height and/or width of a recess or the protrusion may be determined by the mechanical strength of the material used to ensure that the recess or the protrusion may support its own weight without cracking during handling.

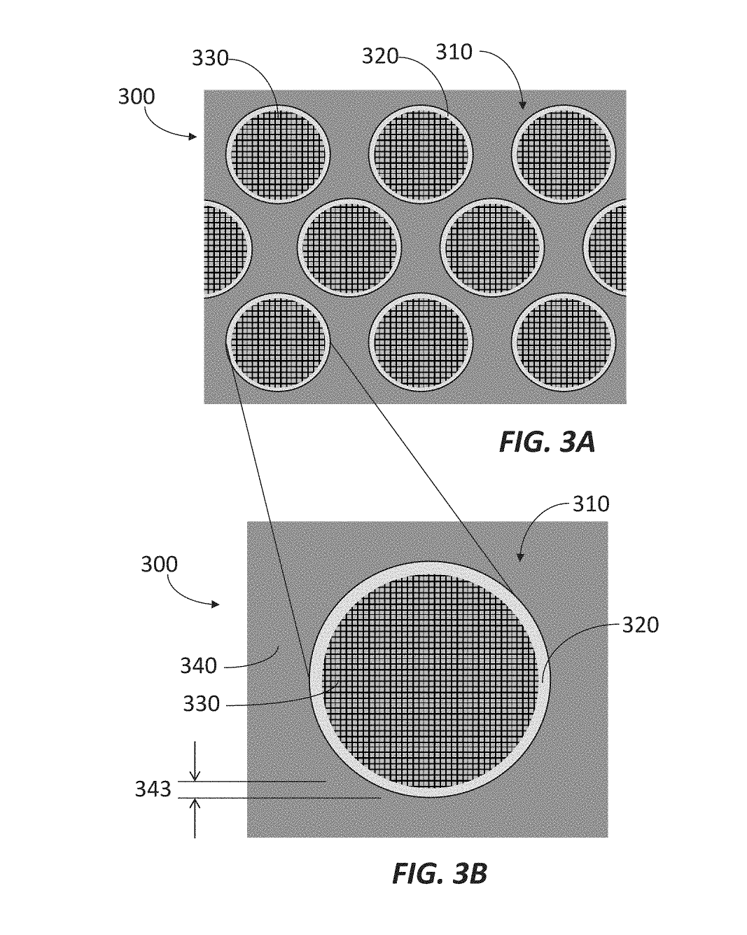

In some embodiments, the surface 340 of the device comprises a textured portion 330, comprising a recess 230b or a protrusion 230b, and an active portion comprising a first molecule 320. In some embodiments, per FIGS. 2A, 2B, 3A, and 3B, the boundary of a textured portion 330 of a locus 310 of a device 200, 300 lies entirely within the boundary of the corresponding locus 210, 310. In some embodiments, the boundary of a textured portion 330 is concentric to or uniformly offset from the boundary of its respective locus 310 by an offset distance 343.

In some instances, a ratio between the offset distance and the diameter of the locus is about 5:1 to about 50:1. In some embodiments, a ratio between the offset distance and the diameter of the loci is at least about 5:1. In some embodiments, a ratio between the offset distance and the diameter of the loci is at most about 50:1. In some embodiments, a ratio between the offset distance and the diameter of the loci is about 5:1 to about 10:1, about 5:1 to about 15:1, about 5:1 to about 20:1, about 5:1 to about 25:1, about 5:1 to about 30:1, about 5:1 to about 35:1, about 5:1 to about 40:1, about 5:1 to about 45:1, about 5:1 to about 50:1, about 10:1 to about 15:1, about 10:1 to about 20:1, about 10:1 to about 25:1, about 10:1 to about 30:1, about 10:1 to about 35:1, about 10:1 to about 40:1, about 10:1 to about 45:1, about 10:1 to about 50:1, about 15:1 to about 20:1, about 15:1 to about 25:1, about 15:1 to about 30:1, about 15:1 to about 35:1, about 15:1 to about 40:1, about 15:1 to about 45:1, about 15:1 to about 50:1, about 20:1 to about 25:1, about 20:1 to about 30:1, about 20:1 to about 35:1, about 20:1 to about 40:1, about 20:1 to about 45:1, about 20:1 to about 50:1, about 25:1 to about 30:1, about 25:1 to about 35:1, about 25:1 to about 40:1, about 25:1 to about 45:1, about 25:1 to about 50:1, about 30:1 to about 35:1, about 30:1 to about 40:1, about 30:1 to about 45:1, about 30:1 to about 50:1, about 35:1 to about 40:1, about 35:1 to about 45:1, about 35:1 to about 50:1, about 40:1 to about 45:1, about 40:1 to about 50:1, or about 45:1 to about 50:1. In some embodiments, a ratio between the offset distance and the diameter of the loci is about 5:1, about 10:1, about 15:1, about 20:1, about 25:1, about 30:1, about 35:1, about 40:1, about 45:1, or about 50:1.

The surface density of texture features on the surface of the device may be about 1 feature/mm² to about 800 features/mm², or greater. The number of texture features per loci may be about 10 to about 50,000. In some embodiments, the number of texture features per loci is about 10, about 100, about 500, about 1,000, about 2,000, about 4,000, about 6,000, about 8,000, about 10,000, about 20,000, about 40,000, or about 50,000.

In some instances, a ratio between the height and the width of a recess or protrusion is about 1:50 to about 50:1. In some instances, the ratio between the height and the width of a recess or protrusion is about 1:50, about 1:20, about 1:10, about 1:5, about 1:2, about 1:1, about 2:1, about 5:1, about 10:1, about 20:1 or about 50:1.

In some instances, the ratio between the height of a recess or a protrusion and the pitch between adjacent recesses or protrusions is about 1:50 to about 50:1. In some instances, the ratio between the height of a recess or a protrusion and the pitch between adjacent recesses or a protrusions is about 1:50, about 1:20, about 1:10, about 1:5, about 1:2, about 1:1, about 2:1, about 5:1, about 10:1, about 20:1 or about 50:1. In some instances, the ratio of height to pitch is about 0.6:1 to about 5:2.

In some instances, the height of a recess or protrusion is about 10 nm to about 1,000,000 nm. In some instances, the height of a recess or protrusion is about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 10 nm to about 50,000 nm, about 10 nm to about 100,000 nm, about 10 nm to about 500,000 nm, about 10 nm to about 1,000,000 nm, about 50 nm to about 100 nm, about 50 nm to about 500 nm, about 50 nm to about 1,000 nm, about 50 nm to about 5,000 nm, about 50 nm to about 10,000 nm, about 50 nm to about 50,000 nm, about 50 nm to about 100,000 nm, or about 50 nm to about 500,000 nm.

In some instances, the width of a recess or protrusion is about 10 nm to about 1,000,000 nm. In some embodiments, the width of a recess or protrusion is at least about 10 nm. In some embodiments, the width of a recess or protrusion is at most about 1,000,000 nm. In some embodiments, the width of a recess or protrusion is about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 10 nm to about 50,000 nm, about 10 nm to about 100,000 nm, about 10 nm to about 500,000 nm, about 10 nm to about 1,000,000 nm, about 50 nm to about 100 nm, about 50 nm to about 500 nm, about 50 nm to about 1,000 nm, about 50 nm to about 5,000 nm, about 50 nm to about 10,000 nm, about 50 nm to about 50,000 nm, about 50 nm to about 100,000 nm, about 50 nm to about 500,000 nm, or about 50 nm to about 1,000,000 nm. In some instances, the width of a recess or protrusion is about 10 nm, about 50 nm, about 100 nm, about 500 nm, about 1,000 nm, about 5,000 nm, about 10,000 nm, about 50,000 nm, about 100,000 nm, about 500,000 nm, or about 1,000,000 nm.

In some instances, the pitch between adjacent recesses or protrusions is about 10 nm to about 1,000,000 nm. In some instances, the pitch between adjacent recesses or protrusions is about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 10 nm to about 50,000 nm, about 10 nm to about 100,000 nm, about 10 nm to about 500,000 nm, about 10 nm to about 1,000,000 nm, about 50 nm to about 100 nm, about 50 nm to about 500 nm, about 50 nm to about 1,000 nm, about 50 nm to about 5,000 nm, about 50 nm to about 10,000 nm, about 50 nm to about 50,000 nm, about 50 nm to about 100,000 nm, about 50 nm to about 500,000 nm, or about 50 nm to about 1,000,000 nm. In some instances, the pitch between adjacent recesses or protrusions is about 10 nm, about 50 nm, about 100 nm, about 500 nm, about 1,000 nm, about 5,000 nm, about 10,000 nm, about 50,000 nm, about 100,000 nm, about 500,000 nm, or about 1,000,000 nm.

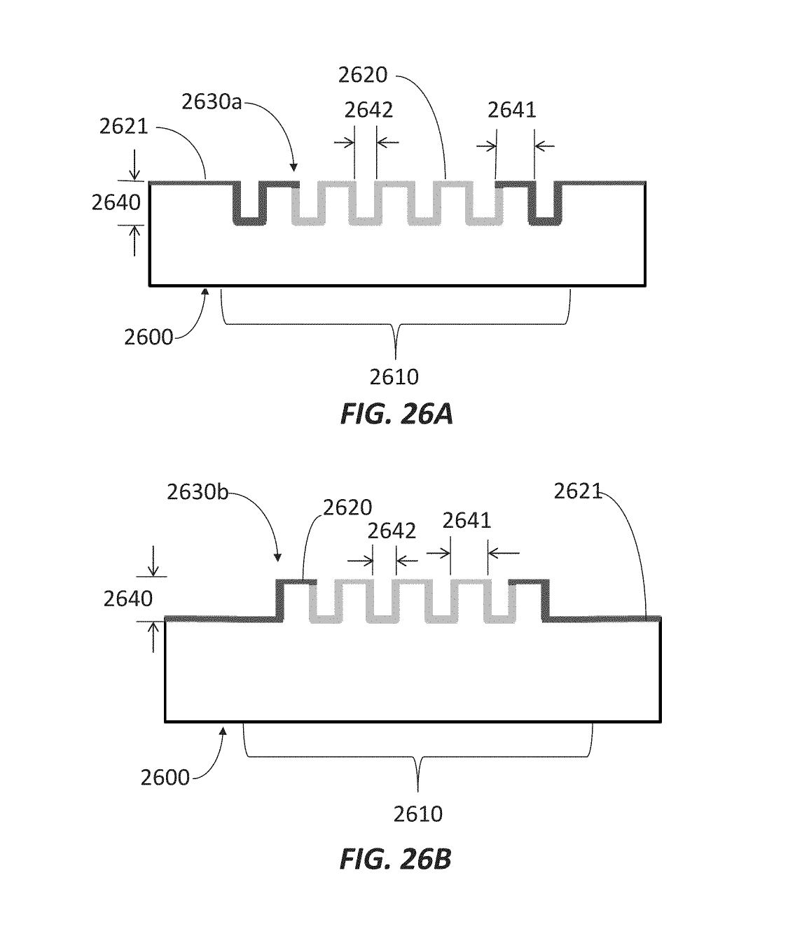

In a second arrangement, devices described herein comprise loci for polynucleotide extension comprising recesses or protrusions, wherein the loci extend beyond a boundary of the recesses or protrusions. In some instances, the surface of the device 2600 comprises a plurality of loci 2610, wherein each locus 2610 comprises a recessed texture 2630a feature, or a raised texture feature 2630b, per FIGS. 26A and 26B, respectively, and an active region 2620. In some instances, the active region 2620 of each locus 2610 is surrounded by a passive region 2621.

In some instances, each recessed texture feature 2630a or raised texture feature 2630b has a depth 2640 and a width 2641, wherein the recessed texture 2630a features, or the raised texture features 2630b are separated by a pitch 2642. A recessed texture feature 2630a or a raised texture feature 2630b may have the same or different depth 2640, width 2641, volume, or any combination thereof as another recessed texture feature 2630b or a raised texture feature 2630b within the same locus 2610. The recessed texture features 2630a or raised texture features 2630b of a locus 2610 may have the same or different depth 2640, width 2641, volume, or any combination thereof as the recessed texture features 2630a or the raised texture features 2630b of another locus 2610. The recessed texture features 2630a or raised texture features 2630b of the loci 2610 within a cluster of loci 2610 may have the same or different depth 2640, width 2641, volume, or any combination thereof as the recessed texture features 2630a or the raised texture features 2630b of the loci 2610 within another cluster of loci 2610.

### Methods of Forming Textured Surfaces

Provided herein is a method for forming a device comprising a surface, wherein the surface is modified to support polynucleotide synthesis at predetermined locations and with a resulting low error rate, a low dropout rate, a high yield, and a high oligo representation. In some embodiments, the surface is modified to support polynucleotide synthesis at predetermined locations. A common method for functionalization comprises selective deposition of an organosilane molecule onto a surface of a device disclosed herein. Selective deposition refers to a process that produces two or more distinct areas on a device, wherein at least one area has a different surface or chemical property that another area of the same device. Such properties include, without limitation, surface energy, chemical termination, surface concentration of a chemical molecule, and the like. Any suitable process that changes the chemical properties of the surface described herein or known in the art may be used to functionalize the surface, for example chemical vapor deposition of an organosilane. Typically, this results in the deposition of a self-assembled monolayer (SAM) of the functionalization species.

Provided herein are methods for functionalizing a surface of a device disclosed herein for polynucleotide synthesis that includes photolithography. An exemplary photolithographic method comprises: 1) applying a photoresist to a surface; 2) exposing the resist to light (e.g., using a binary mask opaque in some areas and clear in others); and 3) developing the resist; wherein the areas that were exposed are patterned. The patterned resist may then serve as a mask for subsequent processing steps, for example, etching, ion implantation, and deposition. After processing, the resist is typically removed, for example, by plasma stripping or wet chemical removal. Oxygen plasma cleaning may optionally be used to facilitate the removal of residual organic contaminants in resist cleared areas, for example, by using a typically short plasma cleaning step (e.g., oxygen plasma). Resist may be stripped by dissolving it in a suitable organic solvent, plasma etching, exposure and development, etc., thereby exposing the areas of the surface that had been covered by the resist. Resist may be removed in a process that does not remove functionalization groups or otherwise damage the functionalized surface.

Provided herein is a method for functionalizing a surface of a device disclosed herein for polynucleotide synthesis comprises a resist or photoresist coat. Photoresist, in many cases, refers to a light-sensitive material useful in photolithography to form patterned coatings. It is applied as a liquid to solidify on a surface as volatile solvents in the mixture evaporate. In some cases, the resist is applied in a spin coating process as a thin film, e.g., 1 μm to 100 μm. The coated resist may be patterned by exposing it to light through a mask or reticle, changing its dissolution rate in a developer. In some cases, the resist coat is used as a sacrificial layer that serves as a blocking layer for subsequent steps that modify the underlying surface, e.g., etching, and then is removed by resist stripping. A surface of a device may be functionalized while areas covered in resist are protected from active or passive functionalization.

Provide herein are methods where a chemical cleaning is a preliminary step in surface preparation. In some exemplary methods, active functionalization is performed prior to lithography. A device may be first cleaned, for example, using a piranha solution. An example of a cleaning process includes soaking a device in a piranha solution (e.g., 90% H₂SO₄, 10% H₂O₂) at an elevated temperature (e.g., 120° C.) and washing (e.g., water) and drying the device (e.g., nitrogen gas). The process optionally includes a post piranha treatment comprising soaking the piranha treated device in a basic solution (e.g., NH₄OH) followed by an aqueous wash (e.g., water). Alternatively, a device may be plasma cleaned, optionally following the piranha soak and optional post piranha treatment. An example of a plasma cleaning process comprises an oxygen plasma etch.

Provided herein are methods for surface preparation where, an active chemical vapor (CVD) deposition step is done after photolithography. An exemplary first step includes optionally cleaning the surface cleaning the surface of a device using cleaning methods disclosed herein. Cleaning may include oxygen plasma treatment. In some cases, the CVD step is for deposition of a mixture, the mixture having at least two molecules resulting in a high surface energy region and the region coated with the first chemical layer is a lower surface energy region. The mixture may comprise a molecule that binds the surface and couple nucleoside phosphoramidite mixed with a greater amount of a molecule that binds the surface and does not couple nucleoside phosphoramidite. For the two-step dilution protocol, prior to depositing the mixture on the surface, a step includes deposition of 100% of the mixture ingredient molecule that binds the surface and does not couple nucleoside phosphoramidite. The first chemical layer may comprise a fluorosilane disclosed herein, for example, tridecafluoro-1,1,2,2-tetrahydrooctyl)-trichlorosilane. The second chemical layer may comprise at least two silanes disclosed herein. In some cases, the two silanes are GOPS and propyltrimethoxysilane. In an exemplary method, the surface is treated with propyltrimethoxysilane prior to treatment with the mixture. The above workflow is an example process and any step or component may be omitted or changed in accordance with properties desired of the final functionalized surface.

A surface of a device disclosed herein may be coated with a resist, subject to functionalization and/or after lithography, and then treated to remove the resist. In some cases, the resist is removed with a solvent, for example, with a stripping solution comprising N-methyl-2-pyrrolidone. In some cases, resist stripping comprises sonication or ultrasonication. After stripping resist, the surface may be further subjected to deposition of an active functionalization agent binding to exposed areas to create a desired differential functionalization pattern. In some cases, the active functionalization areas comprise one or more different species of silanes, for example, 1, 2, 3, 4, 5, 6, 7, 8, 9, 10 or more silanes. One of the one or more silanes may be present in the functionalization composition in an amount greater than another silane. The composition and density of functionalization agent may contribute to a low error rate of polynucleotide synthesis, e.g., an error rate of less than 1 in 1000, less than 1 in 1500, less than 1 in 2000, less than 1 in 3000, less than 1 in 4000, less than 1 in 5000 bases).

Provided herein are methods which include applying an adhesion promoter to the surface. The adhesion promoter is applied in addition to applying the light sensitive lack. In some cases, applying both the adhesion promoter and light sensitive lack is done to surfaces including, without limitation, glass, silicon, silicon dioxide, and silicon nitride. Exemplary adhesion promoters include silanes, e.g., aminosilanes. Exemplary aminosilanes include, without limitation, (3-aminopropyl)trimethoxysilane, (3-aminopropyl) triethoxysilane, and N-(3-triethoxysilylpropyl)-4-hydroxybutyramide. In addition, a passive layer may be deposited on the surface, which may or may not have reactive oxide groups. The passive layer may comprise silicon nitride (Si₃N₄) or polyamide. The photolithographic step may be used to define regions where loci form on the passivation layer.

Provided here are methods for producing a substrate having a plurality of loci starts with a device. The device composed of a material (e.g., silicon) may have any number of layers disposed upon it, including but not limited to a conducting layer such as a metal (e.g., silicon dioxide, silicon oxide, or aluminum). A surface of the device may comprise a protective layer (e.g., titanium nitride). The layers may be deposited with the aid of various deposition techniques, such as, for example, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD) and physical vapor deposition (e.g., sputter deposition, evaporative deposition). In some cases, a layer may be deposited via plasma enhanced CVD (PECVD). A layer of thermal oxide may serve as an etch mask for the silicon. In some cases, this layer of thermal oxide may have a thickness of at least about 1 nm, 2 nm, 3 nm, 4 nm, 5 nm, 6 nm, 7 nm, 8 nm, 9 nm, 10 nm, 15 nm, 20 nm, 50 nm, 60 nm, 70 nm, 80 nm, 90 nm, 100 nm, 200 nm, 300 nm, 400 nm, 500 nm, 600 nm, 700 nm, 800 nm, 900 nm, or more.

An oxide layer may be deposited on the surface of the device provided herein. The oxide layer may comprise silicon dioxide. The silicon dioxide may be deposited using tetraethyl orthosilicate (TEOS), high density plasma (HDP), or any combination thereof. The silicon dioxide may be deposited to a thickness suitable for the manufacturing of suitable microstructures described in further detail elsewhere herein. In some cases, silicon dioxide is grown in a conformal way on a silicon substrate. Growth on a silicon substrate may be performed in a wet or dry atmosphere. An exemplary wet growth method is provided where wet growth is conducted at high temperatures, e.g., about 1000 degrees Celsius and in water vapor. The dry growth method may be conducted in the presence of oxygen.

Loci may be created using photolithographic techniques such as those used in the semiconductor industry. For example, a photo-resist (e.g., a material that changes properties when exposed to electromagnetic radiation) may be coated onto the silicon dioxide (e.g., by spin coating of a wafer) to any suitable thickness. Exemplary coating thicknesses include about 1, 5, 10, 20, 30, 40, 50, 60, 70, 80, 90, 100, 150, 200, 300, 400, or 500 μm. An exemplary photoresist material is MEGAPOSIT SPR 3612 photoresist (Dow Electronic Material) or a similar product. The substrate including the photo-resist may be exposed to an electromagnetic radiation source. A mask may be used to shield radiation from portions of the photo-resist in order to define the area of the loci. The photo-resist may be a negative resist or a positive resist (e.g., the area of the loci may be exposed to electromagnetic radiation or the areas other than the loci may be exposed to electromagnetic radiation as defined by the mask). The area overlying the location in which the resolved loci are to be created is exposed to electromagnetic radiation to define a pattern that corresponds to the location and distribution of the resolved loci in the silicon dioxide layer. The photoresist may be exposed to electromagnetic radiation through a mask defining a pattern that corresponds to the resolved loci. Next, the exposed portion of the photoresist may be removed, such as, e.g., with the aid of wet chemical etching and a washing operation. The removed portion of the mask may then be exposed to a chemical etchant to etch the substrate and transfer the pattern of resolved loci into the silicon dioxide layer. The etchant may include an acid, such as, for example, buffered HF in the case of silicon dioxide.

Various etching procedures may be used to etch the silicon in the area where the resolved loci are to be formed. The etching procedure may be an isotropic etch (i.e., the etching rate alone one direction substantially equal or equal to the etching rate along an orthogonal direction), or an anisotropic etch (i.e., the etching rate along one direction is less than the etching rate alone an orthogonal direction), or variants thereof. The etching techniques may be both wet silicon etches such as KOH, TMAH, EDP and the like, and dry plasma etches (for example DRIE). Both may be used to etch microstructures wafer through interconnections.

The dry etch may be an anisotropic etch that etches substantially vertically (e.g., toward the substrate) but not laterally or substantially laterally (e.g., parallel to the substrate). In some cases, the dry etch comprises etching with a fluorine based etchant such as CF₄, CHF₃, C₂F₆, C₃ F₆, or any combination thereof. In some cases, the etching is performed for 400 seconds with an Applied Materials eMax-CT machine having settings of 100 mT, 1000 W, 20 G, and 50 CF4. The substrates described herein may be etched by deep reactive-ion etching (DRIE). DRIE is a highly anisotropic etching process used to create deep penetration, steep-sided holes, and trenches in wafers/substrates, typically with high aspect ratios. The substrates may be etched using two main technologies for high-rate DRIE: cryogenic and Bosch. Methods of applying DRIE are described in the U.S. Pat. No. 5,501,893, which is herein incorporated by reference in its entirety.

The wet etch may be an isotropic etch that removes material in all directions. In some cases, the wet oxide etches are performed at room temperature with a hydrofluoric acid base that may be buffered (e.g., with ammonium fluoride) to slow down the etching rate. In some cases, a chemical treatment may be used to etch a thin surface material, e.g., silicon dioxide or silicon nitride. Exemplary chemical treatments include buffered oxide etch (BOE), buffered HF and/or NH₄F. The etching time needed to completely remove an oxide layer is typically determined empirically. In one example, the etching is performed at 22° C. with 15:1 BOE (buffered oxide etch).

The silicon dioxide layer may be etched up to an underlying material layer. For example, the silicon dioxide layer may be etched until a titanium nitride layer. In some cases, the silicon dioxide is grown at a temperature of 1000 degrees Celsius and the underlying layer is typically silicon.

An additional surface layer may be added on top of an etched silicon layer subsequent to etching. In an exemplary arrangement, the additional surface layer is one that effectively binds to an adhesion promoter. Exemplary additional surface layers include, without limitation, silicon dioxide and silicon nitride. In the case of silicon dioxide, the additional layer may be added by conformal growth of a thin layer of this material on the silicon.

In some cases, photolithography is applied to the surface of the device herein to create a mask of photoresist. In a subsequent step, a deep reactive-ion etching (DRIE) step is used to etch vertical sidewalls (e.g., until an insulator layer in a structure comprising an insulator layer) at locations devoid of the photoresist. In a following step, the photoresist is stripped. Photolithography, DRIE, and photoresist strip steps may be repeated on the device handle side. In cases wherein the surface of the device comprises an insulator layer such as silicon dioxide, buried oxide (BOX) is removed using an etching process. Thermal oxidation may then be applied to remove contaminating polymers that may have been deposited on the sidewalls during the method. In a subsequent step, the thermal oxidation is stripped using a wet etching process.

To resist coat only a small region of the surface (e.g., lowered features such as a well and/or channel), a droplet of resist may be deposited into the lowered feature where it optionally spreads. In some cases, a portion of the resist is removed, for example, by etching (e.g., oxygen plasma etch) to leave a smooth surface covering only a select area.

The surface may be wet cleaned, for example, using a piranha solution. Alternatively, the surface may be plasma cleaned, for example, by dry oxygen plasma exposure. The photoresist may be coated by a process governed by wicking into the device layer channels. The photoresist may be patterned using photolithography to expose areas that are desired to be passive (i.e., areas where polynucleotide synthesis is not designed to take place). Patterning by photolithography may occur by exposing the resist to light through a binary mask that has a pattern of interest. After exposure, the resist in the exposed regions may be removed in developer solution.

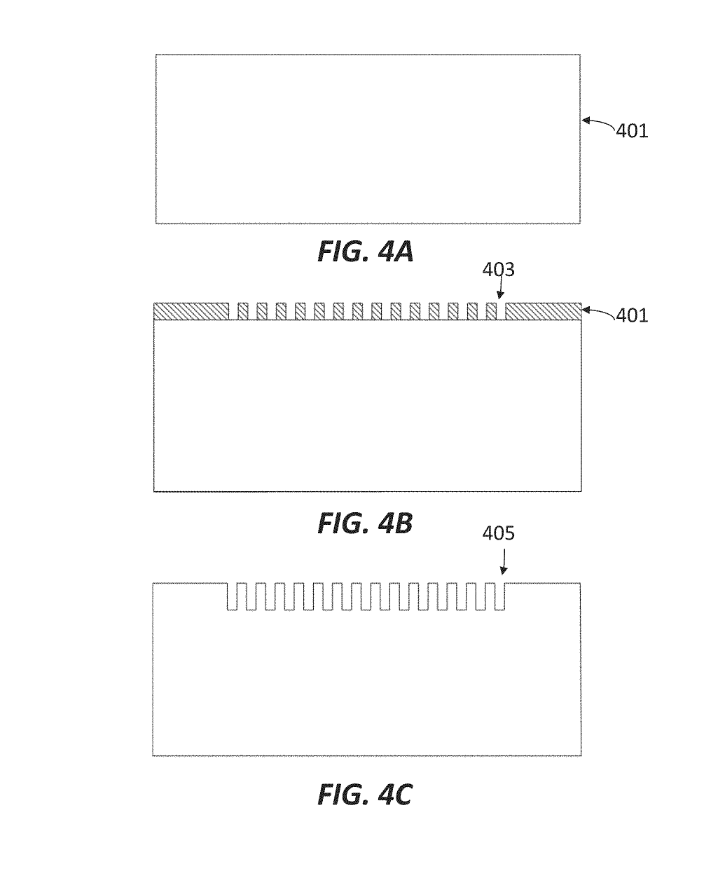

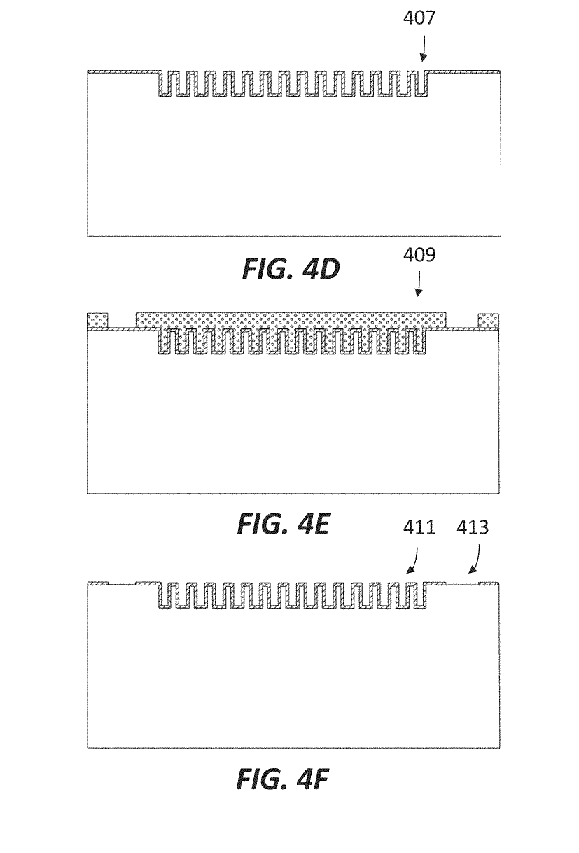

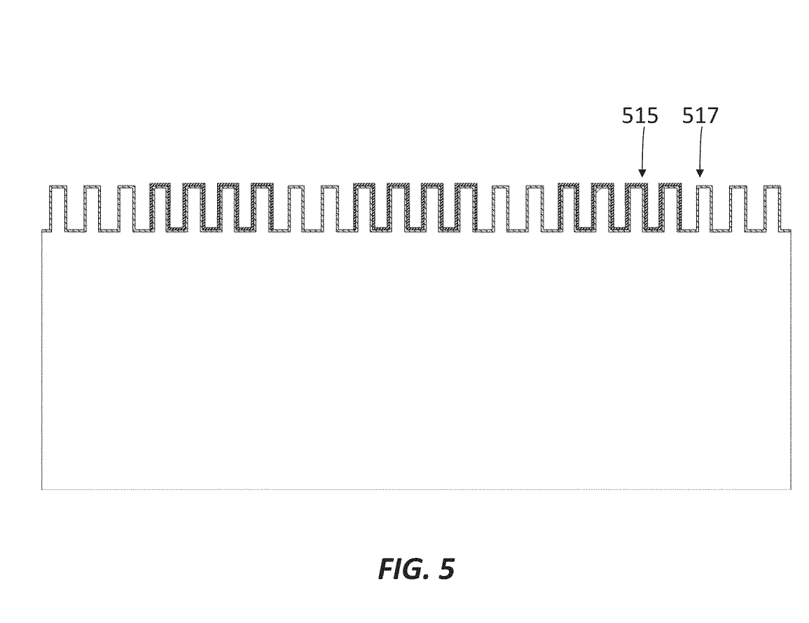

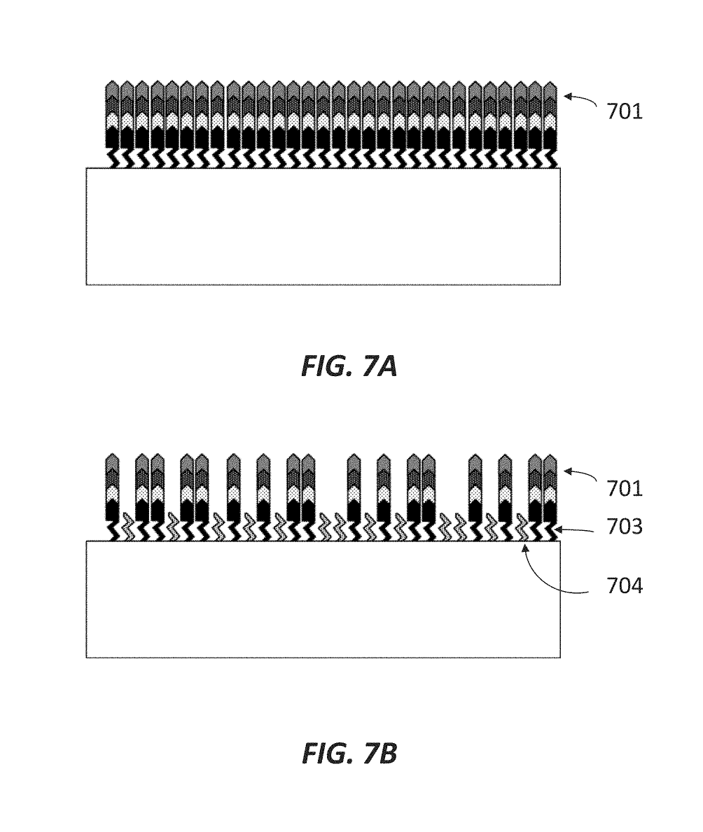

A number of steps are performed to make a textured surface. An exemplary device 401 (e.g., silicon-based) disclosed herein is polished (FIG. 4A), a textured layer pattern 401 is formed via printing lithography (FIG. 4B), a silicon reactive ion etching and resist strip is performed to leave indents 403 in the surface (FIG. 4C), the surface is subject to oxidation 407 (FIG. 4D), a fiducial layer is optionally printed on via lithography using a photosensitive lack 409 (FIG. 4E), after which a final oxide etching results in a textured silicon surface having a fiducial structure 413 (FIG. 4F). The device may then be additionally exposed to functionalization agents as described above and elsewhere herein, to result in a device having a patterned surface with loci coated with a molecule for coupling to a nucleoside 515 (alone or as a mixture depicted in FIG. 7B) surrounding by regions coated with an agent that does not couple a nucleoside 517, FIG. 5.

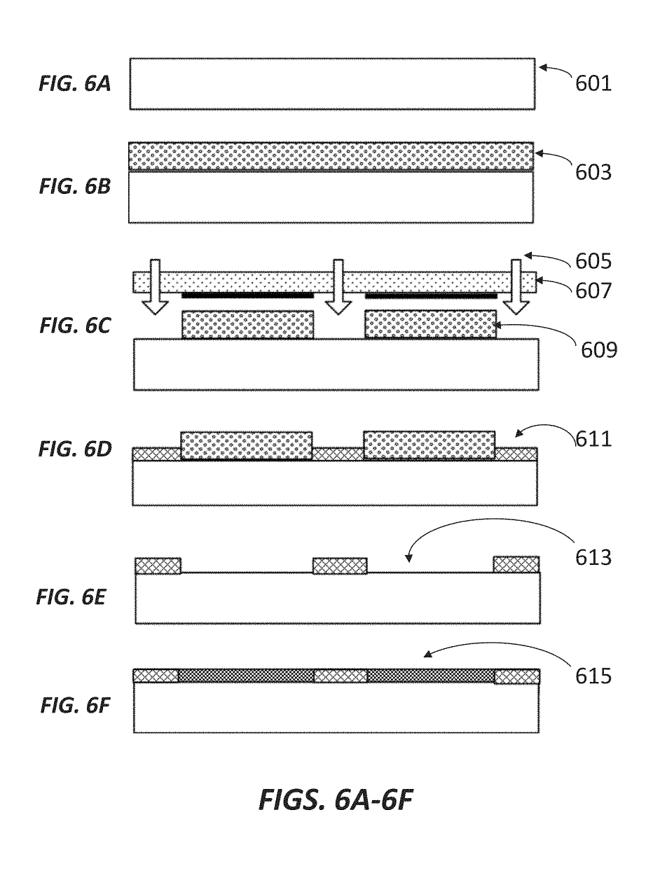

An exemplary method for generating a surface having a reduction in nucleoside-coupling agent density is illustrated in FIGS. 6A-6F. As a first step, a device 601 is optionally cleaned with oxygen plasma, FIG. 6A. Exemplary devices include those made of silicon dioxide or silicon oxide. Directly after cleaning, the device is coated with a photosensitive lack 603 (e.g., photoresist), FIG. 6B. Optical lithography is then performed, FIG. 6C, where electromagnetic wavelength 605 is projected through a shadow mask 607, resulting in removal of the photosensitive lack at predetermined locations and remaining photosensitive lack 609 at other locations. Next, a first molecule (e.g., a fluorosilane) is deposited on the surface and coats the surface at regions exposed as a result of photolithography 611, FIG. 6D. The first molecule is one that does not couple to nucleoside. The photosensitive lack is then stripped away (FIG. 6E), revealing exposed regions 613. The next step involves a two-part deposition process. First, a second molecule that binds the surface and lacks reactive group capable of binding to a nucleoside. Next, a mixture is deposited on the surface comprising the second molecule, and a third molecule, where the third molecule binds the surface and is also able to couple nucleoside, FIG. 6F. This two-step deposition process results in predetermined sites 615 on the surface having a low concentration of activating agent. To assist with efficiency of the reactions during the polynucleotide synthesis process, the region for polynucleotide extension (a locus) has a higher surface energy than the region of the surface surrounding the locus.

When polynucleotides 701 are extended from a surface having a saturating amount of nucleoside-coupling molecule, they are relatively crowded, FIG. 7A. In contrast, when polynucleotides are extended on a surface having a mixture of a nucleoside-coupling molecule 703 and a non-nucleoside-coupling molecule 704, less crowding results, FIG. 2B, and a lower error rate is observed. A first exemplary method of functionalizing a surface is discussed above with reference to FIGS. 6A-1F. In FIG. 6, the active functionalizing agent is deposited as a last step, FIG. 6F.

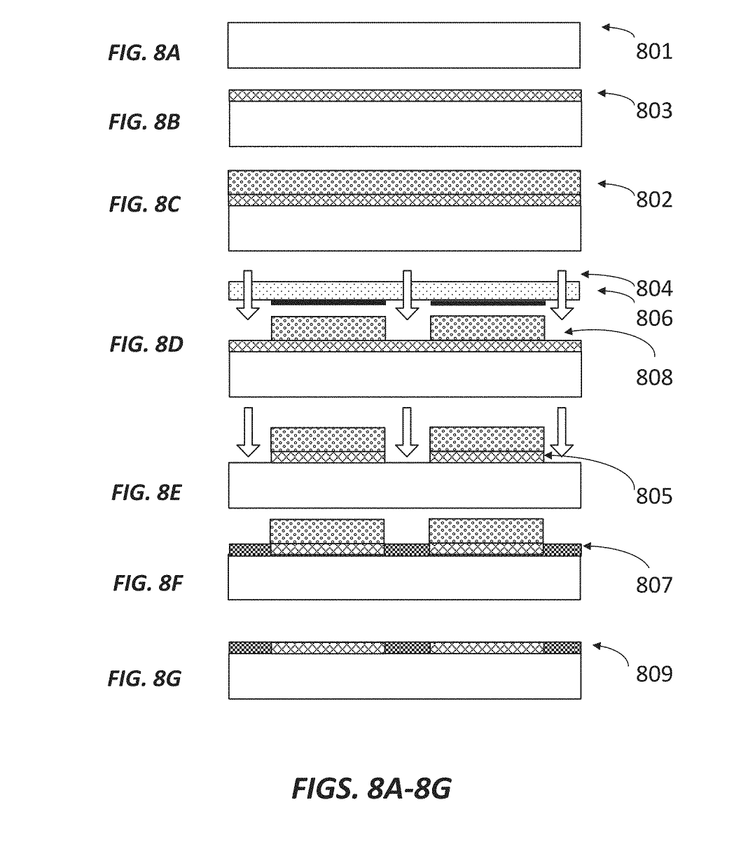

Alternatively, the active functionalization agent may be deposited earlier in the process and function as an adhesion promoter for a photosensitive lack. FIG. 8 provides an illustrative representation of this alternative method. As a first step, a device 801 is optionally cleaned with oxygen plasma, FIG. 8A. After cleaning, a surface of the device is coated with a first chemical layer, a molecule that binds the surface and binds nucleoside is deposited on the surface 803 (e.g., an aminosilane), FIG. 8B. The surface of the device is then coated with a photosensitive lack 803, FIG. 8C. Optical lithography is then performed, FIG. 8D, where electromagnetic wavelength 804 is projected through a shadow mask 806, resulting in removal of the photosensitive lack 802 at predetermined locations and remaining photosensitive lack 808 (e.g., photoresist) at other locations. The use of a photoresist mask results in patterning of the first chemical layer 805, FIG. 8E. A second chemical layer, a molecule that binds the surface and does not bind nucleoside 807, is deposited on the surface, FIG. 8F. The photosensitive lack is then stripped away (FIG. 8G), revealing patterned regions 809. The resulting surface is patterned with loci comprising nucleoside-coupling molecules for polynucleotide extension reactions.

Provided herein are devices for polynucleotide synthesis, comprising a plate having a surface; a plurality of loci on the surface; and a plurality of recesses or protrusions spanning the region of each locus, wherein each recess or protrusion has a width that is about 200 to 500 nm in length, and wherein each recess or protrusion has a depth that is about 250 to 1000 nm in length. Further provided here are devices, wherein each recess or protrusion has a width that is 200 nm in length. Further provided here are devices, wherein each recess or protrusion has a depth that is 250 to 500 nm in length. Further provided here are devices, wherein each recess or protrusion has a depth that is 500 nm in length. Further provided here are devices, wherein each locus has a pitch of 400 to 1000 nm in length. Further provided here are devices, wherein each locus has a pitch of 400 nm in length. Further provided here are devices, wherein each locus has a diameter of about 0.5 to 100 μm in length. Further provided here are devices, wherein each locus comprises a plurality of first molecules, and wherein the plurality of first molecules comprises a first molecule that binds to the surface and comprises a reactive group capable of binding to a nucleoside. Further provided here are devices, wherein the first molecule silane. Further provided here are devices, wherein the silane is an aminosilane. Further provided here are devices, wherein the first molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Further provided here are devices, wherein a region surrounding each locus comprises a plurality of second molecules, wherein the plurality of second molecules comprises a second molecule that binds to the surface and lacks the reactive group capable of binding to the nucleoside. Further provided here are devices, wherein the second molecule is a fluorosilane. Further provided here are devices, wherein the fluorosilane is (tridecafluoro-1,1,2,2-tetrahydrooctyl)trichlorosilane, perfluorooctyltrichlorosilane, perfluorooctyltriethoxysilane, or perfluorooctyltrimethoxychlorosilane. Further provided here are devices, further comprising a plurality of clusters on the surface, wherein each cluster comprises a subset of the plurality of loci, and wherein the subset of the plurality of loci comprises 50 to 500 loci. Further provided here are devices, wherein the subset of the plurality of loci comprises 121 loci. Further provided here are devices, wherein each of the clusters has a diameter of 0.5 to 2 mm. Further provided here are devices, wherein each locus comprises a plurality of third molecules, wherein the plurality of third molecules comprises a third molecule that binds to the surface and lacks a reactive group capable of binding to a nucleoside. Further provided here are devices, wherein the third molecule is propyltrimethoxysilane. Provided herein are devices for polynucleotide synthesis, comprising: a silicon wafer having a surface; a plurality of loci on the surface; and a plurality of recesses or protrusions spanning the region of each locus, wherein each recess or protrusion has a width that is about 200 to 400 nm in length, and wherein each recess or protrusion has a depth that is about 250 to 500 nm in length. Provided herein are systems polynucleotide synthesis, comprising: a material deposition device comprising plurality of reagents for polynucleotide synthesis and a plurality of nozzles for depositing the plurality of reagents for polynucleotide synthesis; a computer for controlling the release of the plurality of reagents for polynucleotide synthesis from the plurality of nozzles; and a plate disclosed herein for receiving the plurality of reagents for polynucleotide synthesis.

Provided herein are methods for polynucleotide synthesis, comprising: providing predetermined sequences for at least 30,000 non-identical polynucleotides; providing the device of any one of claims 1 to 20; synthesizing the at least 30,000 non-identical polynucleotides, wherein each of the at least 30,000 non-identical oligonucleic is at least 30 bases in length and extends from different locus on the surface, and wherein the at least 30,000 non-identical polynucleotides encode sequences with an aggregate error rate of less than 1 in 1000 bases compared to the predetermined sequences. Further provided here are methods, further comprising releasing the at least 30,000 non-identical polynucleotides from the surface; and assembling at least 250 preselected nucleic acids, wherein the assembled at least 250 preselected nucleic acids encode sequences with an aggregate deletion error rate of less than 1 in 1000 bases compared to the predetermined sequences.

Provided herein are methods for polynucleotide synthesis, comprising: providing a device comprising a surface, wherein the device comprises a plurality of loci on the surface; and a plurality of recesses or protrusions spanning the region of each locus, wherein each recess or protrusion has a width that is about 200 to 400 nm in length, and wherein each recess or protrusion has a depth that is about 250 to 500 nm in length; providing predetermined sequences for at least 5,000 non-identical polynucleotides; and synthesizing the at least 5,000 non-identical polynucleotides, wherein each of the at least 5,000 non-identical polynucleotides is at least 30 bases in length and extends from the surface, and wherein the at least 5,000 non-identical polynucleotides encode sequences with an aggregate error rate of less than 1 in 1000 bases compared to the predetermined sequences without correcting errors.

Provided herein are methods for polynucleotide synthesis, comprising: providing a device comprising a surface, wherein the device comprises a plurality of loci on the surface; and a plurality of recesses or protrusions spanning the region of each locus, wherein each recess or protrusion has a width that is about 200 to 500 nm in length, and wherein each recess or protrusion has a depth that is about 250 to 1000 nm in length; depositing a first plurality of molecules on the surface at a first region, wherein the first region comprises a plurality of loci, and wherein a first plurality of molecules comprises a first molecule that binds to the surface and lacks a reactive group capable of binding to a nucleoside; and depositing a mixture on the surface at the first region, wherein the mixture comprises the first plurality of molecules and a second plurality of molecules, wherein the second plurality of molecules comprises a second molecule, wherein the second molecule binds to the surface and comprises a reactive group capable of binding the nucleoside, and wherein the first molecule and the second molecule are present in the mixture in a molar ratio of 10:1 to about 2500:1; providing predetermined sequences for at least 5,000 non-identical polynucleotides; and synthesizing the at least 5,000 non-identical polynucleotides, wherein each of the at least 5,000 non-identical polynucleotides is at least 30 bases in length and extends from the surface, and wherein the at least 5,000 non-identical polynucleotides encode sequences with an aggregate error rate of less than 1 in 1000 bases compared to the predetermined sequences. Further provided here are methods, wherein the at least 5,000 non-identical polynucleotides collectively encode for at least 40 genes. Further provided here are methods, wherein the at least 6,000 non-identical polynucleotides collectively encode for at least 50 genes. Further provided here are methods, wherein the at least 100,000 non-identical polynucleotides collectively encode for at least 750 genes. Further provided here are methods, wherein the second molecule is a silane. Further provided here are methods, wherein the silane is an amino silane. Further provided here are methods, wherein the second molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Further, provided here are methods, the first molecule is propyltrimethoxysilane. The method of claim 24, further comprising depositing a third plurality of molecules on the surface at a second region, wherein the third plurality of molecules comprises a third molecule that binds to the surface and lacks the reactive group capable of binding the nucleoside, wherein the first molecule and the second molecule both have a higher surface energy than a surface energy of the third molecule. Further provided here are methods, wherein a difference in water contact angle between the first region and the second region is at least 10 degrees. Further provided here are methods, wherein the third molecule comprises a fluorosilane. Further provided here are methods, wherein the fluorosilane is (tridecafluoro-1,1,2,2-tetrahydrooctyl)trichlorosilane, perfluorooctyltrichlorosilane, perfluorooctyltriethoxysilane, or perfluorooctyltrimethoxychlorosilane. Further provided here are methods, wherein the mixture comprises the second molecule and the third molecule present in a molar ratio of 50:1 to 2500:1. Further provided here are methods, wherein the mixture comprises the second molecule and the third molecule present in a molar ratio of 2000:1. Further provided here are methods, wherein the surface comprises a layer of silicon dioxide. Further provided here are methods, wherein the at least 5,000 non-identical polynucleotides encode sequences with an aggregate error rate of less than 1 in 1000 bases compared to the predetermined sequences. Further provided here are methods, wherein the at least 5,000 non-identical polynucleotides encode sequences with an aggregate error rate of less than 1 in 2000 bases compared to the predetermined sequences. Further provided here are methods, wherein the at least 5,000 non-identical polynucleotides encode sequences with an aggregate error rate of less than 1 in 3000 bases compared to the predetermined sequences. Further provided here are methods, wherein each of the at least 5,000 non-identical polynucleotides is 30 bases to 200 bases in length. Further provided here are methods, wherein each of the at least 5,000 non-identical polynucleotides is about 50 to about 120 bases in length.

Provided herein are methods for preparing a surface for polynucleotide synthesis, comprising: providing a device comprising a surface, wherein the device comprises silicon dioxide; depositing a first molecule on the surface at a first region, wherein the first molecule binds to the surface and lacks a reactive group that binds to a nucleoside phosphoramidite; depositing a second molecule on the surface at a second region, wherein the second region comprises a plurality of loci surrounded by the first region, wherein the second molecule binds to the surface and lacks a reactive group that binds to the nucleoside phosphoramidite; and depositing a mixture on the surface at the second region, wherein the mixture comprises the second molecule and a third molecule, wherein the third molecule binds to the surface and nucleoside phosphoramidite, and wherein the mixture comprises a greater amount of the second molecule than the third molecule. Methods are further provided wherein the second molecule and the third molecule both have a higher surface energy than a surface energy of the first molecule, and wherein surface energy is a measurement of water contact angle on a smooth planar surface. Methods are further provided wherein the difference in water contact angle between the first region and the second region is at least 10, 20, 50, or 75 degrees. Methods are further provided wherein the third molecule is a silane. Methods are further provided wherein the third molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Methods are further provided wherein the third molecule is 3-glycidoxypropyltrimethoxysilane. Methods are further provided wherein the silane is an amino silane. Methods are further provided wherein the second molecule is propyltrimethoxysilane. Methods are further provided wherein the first molecule is a fluorosilane. Methods are further provided wherein the fluorosilane is (tridecafluoro-1,1,2,2-tetrahydrooctyl)trichlorosilane. Methods are further provided wherein the mixture comprises the second molecule and the third molecule present in a molar ratio of about 100:1 to about 2500:1. Methods are further provided wherein the mixture comprises the second molecule and the third molecule present in a molar ratio of about 2000:1. Methods are further provided wherein the mixture comprises the second molecule and the third molecule present in a molar ratio of 2000:1. Methods are further provided wherein the first molecule lacks a free hydroxyl, amino, or carboxyl group. Methods are further provided wherein the second molecule lacks a free hydroxyl, amino, or carboxyl group. Methods are further provided wherein the mixture is in a gaseous state when deposited on the surface. Methods are further provided wherein the first molecule is in a gaseous state when deposited on the surface. Methods are further provided wherein the surface comprises a layer of silicon oxide. Provided herein is a device for polynucleotide synthesis prepared by any one of the methods described herein.

Provide herein are methods for preparing a surface for polynucleotide synthesis, comprising: providing a device comprising a surface, wherein the device comprises silicon dioxide, and wherein the surface comprises a layer of silicon oxide; coating the surface with a light-sensitive material that binds silicon oxide; exposing predetermined regions of the surface to a light source to remove a portion of the light-sensitive material coated on the surface; depositing a first molecule on the surface, wherein the first molecule binds the surface at the predetermined regions and lacks a reactive group that binds to a nucleoside phosphoramidite; removing a remaining portion of the light-sensitive material coated on the surface to expose loci, wherein each of the loci are surrounded by the predetermined regions comprising the first molecule; depositing a second molecule on the surface at the loci, wherein the second molecule binds to the loci and lacks a reactive group that binds to the nucleoside phosphoramidite; and depositing a mixture on the surface at the loci, the mixture comprises the second molecule and a third molecule, and wherein the third molecule binds to the surface and nucleoside phosphoramidite. Methods are further provided wherein the second molecule and the third molecule both have a higher surface energy than a surface energy of the first molecule, wherein the second molecule and the third molecule both have a higher surface energy than a surface energy of the first molecule, and wherein surface energy is a measurement of water contact angle on a smooth planar surface. Methods are further provided wherein the difference in water contact angle between the first region and the second region is at least 10, 20, 50, or 75 degrees. Methods are further provided wherein the difference in water contact angle between the first region and the second region is at least 50 degrees. Methods are further provided wherein the third molecule is a silane. Methods are further provided wherein the third molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Methods are further provided wherein the third molecule is 3-glycidoxypropyltrimethoxysilane. Methods are further provided wherein the silane is an aminosilane. Methods are further provided wherein the second molecule is propyltrimethoxysilane. Methods are further provided wherein the first molecule is a fluorosilane. Methods are further provided wherein the fluorosilane is (tridecafluoro-1,1,2,2-tetrahydrooctyl)trichlorosilane. Methods are further provided wherein the mixture comprises the second molecule and the third molecule present in a molar ratio of about 100:1 to about 2500:1. Methods are further provided wherein the mixture comprises the second molecule and the third molecule present in a molar ratio of about 2000:1. Methods are further provided wherein the mixture comprises the second molecule and the third molecule present in a molar ratio of 2000:1. Methods are further provided wherein the first molecule lacks a free hydroxyl, amino, or carboxyl group. Methods are further provided wherein the second molecule lacks a free hydroxyl, amino, or carboxyl group. Methods are further provided wherein the mixture is in a gaseous state when deposited on the surface. Methods are further provided wherein the first molecule is in a gaseous state when deposited on the surface. Methods are further provided wherein the method further comprises applying oxygen plasma to the surface prior to coating the surface with the light-sensitive material that binds silicon oxide. Methods are further provided wherein the method further comprises applying oxygen plasma to the surface after exposing predetermined regions of the surface to light. Provided herein is a device for polynucleotide synthesis prepared by any one of the methods described herein.

Provided herein are methods for polynucleotide synthesis, comprising: providing predetermined sequences for at least 30,000 non-identical polynucleotides; providing a device comprising a patterned surface, wherein the device comprises silicon dioxide; wherein the patterned surface is generated by: depositing a first molecule on the surface at a first region, wherein the first molecule binds to the surface and lacks a reactive group that binds to a nucleoside phosphoramidite; and depositing a second molecule on the surface at a second region, wherein the second region comprises a plurality of loci surrounded by the first region, wherein the second molecule binds to the surface and lacks a reactive group that binds to the nucleoside phosphoramidite; and depositing a mixture on the surface at the second region, wherein the mixture comprises the second molecule and a third molecule, wherein the third molecule binds to the surface and nucleoside phosphoramidite, wherein the mixture comprises a greater amount of the second molecule than the third molecule; and synthesizing the at least 30,000 non-identical polynucleotides each at least 10 bases in length, wherein the at least 30,000 non-identical polynucleotides encode sequences with an aggregate deletion error rate of less than 1 in 1000 bases compared to the predetermined sequences, and wherein each of the at least 30,000 non-identical polynucleotides extends from a different locus. Methods are further provided wherein the second molecule and the third molecule both have a higher surface energy than a surface energy of the first molecule, and surface energy is a measurement of water contact angle on a smooth planar surface. Methods are further provided wherein a difference in water contact angle between the first region and the second region is at least 10, 20, 50, or 75 degrees. Methods are further provided wherein the difference in water contact angle between the first region and the second region is at least 50 degrees. Methods are further provided wherein the third molecule is a silane. Methods are further provided wherein the third molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Methods are further provided wherein the silane is an aminosilane. Methods are further provided wherein the third molecule is 3-glycidoxypropyltrimethoxysilane. Methods are further provided wherein the second molecule is propyltrimethoxysilane. Methods are further provided wherein the first molecule is a fluorosilane. Methods are further provided wherein the fluorosilane is (tridecafluoro-1,1,2,2-tetrahydrooctyl)trichlorosilane. Methods are further provided wherein the mixture comprises the second molecule and the third molecule present in a molar ratio of about 100:1 to about 2500:1. Methods are further provided wherein the mixture comprises the second molecule and the third molecule present in a molar ratio of about 2000:1. Methods are further provided wherein the mixture comprises the second molecule and the third molecule present in a molar ratio of 2000:1. Methods are further provided wherein the first molecule lacks a free hydroxyl, amino, or carboxyl group. Methods are further provided wherein the second molecule lacks a free hydroxyl, amino, or carboxyl group. Methods are further provided wherein the mixture is in a gaseous state when deposited on the surface. Methods are further provided wherein the first molecule is in a gaseous state when deposited on the surface. Methods are further provided wherein each of the at least 30,000 non-identical polynucleotides is at least 30 bases in length. Methods are further provided wherein each of the at least 30,000 non-identical polynucleotides is 10 bases to 1 kb in length. Methods are further provided wherein each of the at least 30,000 non-identical polynucleotides is about 50 to about 120 bases in length. Methods are further provided wherein the aggregate deletion error rate is less than about 1 in 1700 bases compared to the predetermined sequences. Methods are further provided wherein the aggregate deletion error rate is achieved without correcting errors. Methods are further provided wherein the at least 30,000 non-identical polynucleotides synthesized encode sequences with an aggregate error rate of less than 1 in 1000 bases compared to the predetermined sequences without correcting errors. Methods are further provided wherein the aggregate error rate is less than 1 in 2000 bases compared to the predetermined sequences. Methods are further provided wherein the aggregate error rate is less than 1 in 3000 bases compared to the predetermined sequences. Methods are further provided wherein the surface comprises a layer of silicon oxide.

Provided herein are methods for nucleic acid synthesis, comprising: providing predetermined sequences for at least 200 preselected nucleic acids; providing a device comprising a patterned surface, wherein the device comprises silicon dioxide; wherein the patterned surface is generated by: depositing a first molecule on the surface at a first region, wherein the first molecule binds to the surface and lacks a reactive group that binds to a nucleoside phosphoramidite; and depositing a second molecule on the surface at a second region, wherein the second region comprises a plurality of loci surrounded by the first region, wherein the second molecule binds to the surface and lacks a reactive group that binds to the nucleoside phosphoramidite; and depositing a mixture on the surface at the second region, wherein the mixture comprises the second molecule and a third molecule, wherein the third molecule binds to the surface and nucleoside phosphoramidite, wherein the mixture comprises a greater amount of the second molecule than the third molecule; and synthesizing at least 20,000 non-identical polynucleotides each at least 50 bases in length, wherein each of the at least 20,000 non-identical polynucleotides extends from a different locus of the patterned surface; releasing the at least 20,000 non-identical polynucleotides from the patterned surface; suspending the at least 20,000 non-identical polynucleotides in a solution; and subjecting the solution comprising at least 20,000 non-identical polynucleotides to a polymerase chain assembly reaction to assemble at least 200 genes, wherein the assembled at least 200 preselected nucleic acids encode sequences with an aggregate deletion error rate of less than 1 in 1500 bases compared to the predetermined sequences. Methods are further provided wherein the second molecule and the third molecule both have a higher surface energy than a surface energy of the first molecule, and wherein surface energy is a measurement of water contact angle on a smooth planar surface. Methods are further provided wherein the difference in water contact angle between the first region and the second region is at least 10, 20, 50, or 75 degrees. Methods are further provided wherein the difference in water contact angle between the first region and the second region is at least 50 degrees. Methods are further provided wherein the third molecule is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Methods are further provided wherein the third molecule is a silane. Methods are further provided wherein the third molecule is 3-glycidoxypropyltrimethoxysilane. Methods are further provided wherein the silane is an aminosilane. Methods are further provided wherein the second molecule is propyltrimethoxysilane. Methods are further provided wherein the first molecule is a fluorosilane. Methods are further provided wherein the fluorosilane is (tridecafluoro-1,1,2,2-tetrahydrooctyl)trichlorosilane. Methods are further provided wherein the mixture comprises the second molecule and the third molecule present in a molar ratio of about 100:1 to about 2500:1. Methods are further provided wherein the mixture comprises the second molecule and the third molecule present in a molar ratio of about 2000:1. Methods are further provided wherein the mixture comprises the second molecule and the third molecule present in a molar ratio of 2000:1. Methods are further provided wherein the first molecule lacks a free hydroxyl, amino, or carboxyl group. Methods are further provided wherein the second molecule lacks a free hydroxyl, amino, or carboxyl group. Methods are further provided wherein each of the at least 20,000 non-identical polynucleotides is about 50 to about 120 bases in length. Methods are further provided wherein the aggregate deletion error rate is less than about 1 in 1700 bases compared to the predetermined sequences. Methods are further provided wherein the aggregate deletion error rate is achieved without correcting errors. Methods are further provided wherein the assembled at least 200 preselected nucleic acids encode sequences with an aggregate error rate of less than 1 in 1000 bases compared to the predetermined sequences without correcting errors. Methods are further provided wherein the aggregate error rate is less than 1 in 2000 bases compared to the predetermined sequences. Methods are further provided wherein the surface comprises a layer of silicon oxide.

Provided here are devices for polynucleotide synthesis, comprising: a device having a surface, wherein the device comprises silicon dioxide; a plurality of recesses or posts on the surface, wherein each recess or post comprises: a width length that is 6.8 nm to 500 nm, a pitch length that is about twice the width length, and a depth length that is about 60% to about 125% of the pitch length; a plurality of loci on the surface, wherein each locus has a diameter of 0.5 to 100 μm, wherein each locus comprises at least two of the plurality of recesses or posts; and a plurality of clusters on the surface, wherein each of the clusters comprise 50 to 500 loci and has a cross-section of 0.5 to 2 mm. Devices are further provided wherein each of the clusters comprise 100 to 150 loci. Devices are further provided wherein the device comprises at least 30,000 loci. Devices are further provided wherein the pitch length is 1 μm or less. Devices are further provided wherein the depth length is 1 μm or less. Devices are further provided wherein each of the loci has a diameter of 0.5 μm. Devices are further provided wherein each of the loci has a diameter of 10 μm. Devices are further provided wherein each of the loci has a diameter of 50 μm. Devices are further provided wherein the cross-section of each of the clusters is about 1.125 mm. Devices are further provided wherein each of the clusters has a pitch of about 1.125 mm. Devices are further provided wherein each locus comprises a molecule that binds to the surface and a nucleoside phosphoramidite. Devices are further provided wherein the molecule that binds to the surface and the nucleoside phosphoramidite is a silane. Devices are further provided wherein the molecule that binds to the surface and the nucleoside phosphoramidite is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane. Devices are further provided wherein the silane is 3-glycidoxypropyltrimethoxysilane. Devices are further provided wherein the silane is an aminosilane. Devices are further provided wherein a region surrounding the plurality of loci comprises a molecule that binds to the surface and lacks a nucleoside phosphoramidite. Devices are further provided wherein the molecule that binds to the surface and lacks the nucleoside phosphoramidite is a fluorosilane. Devices are further provided wherein the fluorosilane is (tridecafluoro-1,1,2,2-tetrahydrooctyl)trichlorosilane or perfluorooctyltrichlorosilane. Devices are further provided wherein the surface comprises a layer of silicon oxide.

Provided herein are methods for polynucleotide synthesis, comprising: providing predetermined sequences; providing a device for polynucleotide synthesis prepared by any one of methods described herein; synthesizing a plurality of non-identical polynucleotides at least 10 bases in length, wherein each of the non-identical polynucleotides extends from a different locus. Methods are further provided wherein the aggregate deletion error rate is less than about 1 in 1700 bases compared to the predetermined sequences. Methods are further provided wherein the aggregate deletion error rate is achieved without correcting errors. Methods are further provided wherein the plurality of non-identical polynucleotides synthesized encode sequences with an aggregate error rate of less than 1 in 1000 bases compared to the predetermined sequences without correcting errors. Methods are further provided wherein the aggregate error rate is less than 1 in 2000 bases compared to the predetermined sequences. Methods are further provided wherein the aggregate error rate is less than 1 in 3000 bases compared to the predetermined sequences.

Provided herein are methods for nucleic acid synthesis, comprising: providing predetermined sequences for at least 200 preselected nucleic acids; providing the device described herein; synthesizing at least 20,000 non-identical polynucleotides each at least 50 bases in length, wherein each of the at least 20,000 non-identical polynucleotides extends from a different locus; releasing the at least 20,000 non-identical polynucleotides from the surface; suspending the at least 20,000 non-identical polynucleotides in a solution; and subjecting the solution comprising at least 20,000 non-identical polynucleotides to a polymerase chain assembly reaction to assemble at least 200 genes, wherein the assembled at least 200 preselected nucleic acids encode sequences with an aggregate deletion error rate of less than 1 in 1500 bases compared to the predetermined sequences. Methods are further provided wherein the aggregate deletion error rate is less than about 1 in 1700 bases compared to the predetermined sequences. Methods are further provided wherein the aggregate deletion error rate is achieved without correcting errors. Methods are further provided wherein the assembled at least 200 preselected nucleic acids encode sequences with an aggregate error rate of less than 1 in 1000 bases compared to the predetermined sequences without correcting errors. Methods are further provided wherein the aggregate error rate is less than 1 in 2000 bases compared to the predetermined sequences.

Provide herein are devices for polynucleotide synthesis, comprising: a device having a surface, wherein the device comprises silicon dioxide; a plurality of recesses or posts on the surface, wherein each recess or post comprises (i) a width length that is 6.8 nm to 500 nm, (ii) a pitch length that is about twice the width length, and (iii) a depth length that is about 60% to about 125% of the pitch length; a plurality of loci on the surface, wherein each locus has a diameter of 0.5 to 100 um, wherein each locus comprises at least two of the plurality of recesses or posts; a plurality of clusters on the surface, wherein each of the clusters comprise 50 to 500 loci and has a cross-section of 0.5 to 2 mm, wherein the plurality of loci comprise a less than saturating amount of a molecule that binds the surface and couples to the nucleoside phosphoramidite; and a plurality of regions surrounding each loci comprise a molecule that binds the surface and does not couple the nucleoside phosphoramidite, wherein the plurality of loci have a higher surface energy than the plurality of regions surrounding each loci. Devices are further provided wherein the molecule that binds the surface and couples to the nucleoside phosphoramidite is a silane disclosed herein. Devices are further provided wherein the molecule that binds the surface and does not couple the nucleoside phosphoramidite is a fluorosilane disclosed herein. Devices are further provided wherein the plurality of loci are coated with a molecule that binds the surface, does not couple the nucleoside phosphoramidite, and has a higher surface energy than the molecule on plurality of regions surrounding each loci. Devices are further provided wherein the surface comprises a layer of silicon oxide.

### Surface Materials

Provided herein is a device comprising a surface, wherein the surface is modified to support polynucleotide synthesis at predetermined locations and with a resulting low error rate, a low dropout rate, a high yield, and a high oligo representation. In some embodiments, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa)m, agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

### Functionalized Surfaces

Provided herein is a device comprising a surface, wherein the surface is modified to support polynucleotide synthesis at predetermined active regions with a resulting low error rate, a low dropout rate, a high yield, and a high oligo representation. In some embodiments, surface characteristics of a surface may be adjusted in various ways that are suitable for oligonucleotide synthesis. Devices disclosed herein are devices comprising a surface, wherein portions of the surface have varying wettability characteristics and varying ability to couple a nucleoside. In some embodiments, a device disclosed herein comprises a surface and a plurality of first molecules deposited on at least a portion of the surface, wherein the plurality of first molecules comprises high-energy molecules that exhibit a high surface energy, wherein the first molecule binds to the surface and comprises a reactive group capable of binding to a nucleoside, and wherein the portion of the surface comprising a first molecule is the active region. In some embodiments, a plurality of second molecules are deposited on a portion of the surface, wherein the plurality of second molecules comprise low-energy molecules that exhibit a low surface energy, wherein the plurality of second molecules bind to the surface and lacks a reactive group that couples a nucleoside, thereby forming a passive region that prevents a coupling reaction to the surface. In some embodiments, the nucleoside comprises a nucleic acid monomer.

In some instances, the plurality of first molecules comprises a mix of various high-energy molecules, wherein each high-energy molecule binds to the surface and comprises a reactive group capable of binding to a nucleoside. In some embodiments, the plurality of first molecules comprises a mix of various high-energy molecules, wherein each high-energy molecule binds to the surface and comprises a reactive group capable of binding to a specific nucleoside to support the synthesis of a certain population of polynucleotides having a certain sequence. In some instances, the polynucleotides synthesized from the surface of the device encode for a longer nucleic acid sequence (e.g., a gene). In some instances, the plurality of second molecules comprises a mix of various low-energy molecules, wherein each low-energy molecule binds to the surface and lacks a reactive group capable of coupling one or more specific nucleosides. In some embodiments, the low-energy molecule comprises a passive functionalization. In some embodiments, low-energy molecules lack an available reactive group, such as a hydroxyl, an amino, or a carboxyl group, to bind to a nucleoside in a coupling reaction.

In some instances, the plurality of first molecules comprises a nucleoside-coupling reactive group, such as nucleoside phosphoramidite. In some embodiments, the reactive group comprises a hydroxyl, an amino, a carboxyl group, or any combination thereof, wherein the reactive group binds to a nucleoside through a coupling reaction. In some cases, the surface of the device comprises silicon and the first molecule comprises an aminosilane molecule, whose silicon atoms bind to the oxygen atoms on the surface, and which employs additional chemical interactions to bind to photoresist or biomolecules. In one example, the first molecule comprises (3-aminopropyl)trimethoxysilane (APTMS) or (3-aminopropyl)triethoxysilane (APTES), wherein each first molecule comprises a silicon atom capable of binding to the oxygen atoms on the surface of the device, and wherein the plurality of first molecules comprise amine groups to bind to the organic molecules. Exemplary first molecules comprise (3-aminopropyl)trimethoxysilane, (3-aminopropyl)triethoxysilane and N-(3-triethoxysilylpropyl)-4-hydroxybutyramide.

The surface energy, or hydrophobicity, of a surface may be correlated to a water contact angle formed between a surface of a drop of water, and the solid surface. A solid surface with a water contact angle of less than 90° is considered to have a high surface area and is termed hydrophilic or polar. A solid surface with a water contact angle of greater than 90° is considered to have a low surface energy and is termed hydrophobic or apolar. Highly hydrophobic surfaces with a very low surface energy have water contact angles of greater than 120°.

In some cases, the difference between the water contact angle of the portions of the surface which comprise a first-molecule and the portions of the surface which comprise a second molecule as measured on one or more smooth or planar equivalent surfaces corresponds to the differential hydrophobicity. In some embodiments, the water contact angle is less than 40°, 35°, 30°, 25°, 20°, 15° or 10°, or is greater than 90°, 85°, 80°, 75°, 70°, 65°, 60°, 55°, 50°, 45°, 40°, 35°, 30°, 25°, 20°, 15° or 10°. In some embodiments, the water contact angle is about 5° to about 90°. In some embodiments, the water contact angle is at least about 5°. In some embodiments, the water contact angle is at most about 90°. In some embodiments, the water contact angle is about 5° to about 10°, about 5° to about 20°, about 5° to about 30°, about 5° to about 40°, about 5° to about 50°, about 5° to about 60°, about 5° to about 70°, about 5° to about 80°, about 5° to about 90°, about 10° to about 20°, about 10° to about 30°, about 10° to about 40°, about 10° to about 50°, about 10° to about 60°, about 10° to about 70°, about 10° to about 80°, about 10° to about 90°, about 20° to about 30°, about 20° to about 40°, about 20° to about 50°, about 20° to about 60°, about 20° to about 70°, about 20° to about 80°, about 20° to about 90°, about 30° to about 40°, about 30° to about 50°, about 30° to about 60°, about 30° to about 70°, about 30° to about 80°, about 30° to about 90°, about 40° to about 50°, about 40° to about 60°, about 40° to about 70°, about 40° to about 80°, about 40° to about 90°, about 50° to about 60°, about 50° to about 70°, about 50° to about 80°, about 50° to about 90°, about 60° to about 70°, about 60° to about 80°, about 60° to about 90°, about 70° to about 80°, about 70° to about 90°, or about 80° to about 90°. In some embodiments, the water contact angle is about 5°, about 10°, about 20°, about 30°, about 40°, about 50°, about 60°, about 70°, about 80°, or about 90°.

Without being bound by theory, the wetting phenomenon is understood to be a measure of the surface tension or attractive forces between molecules at a solid-liquid interface, and is expressed in dynes/cm². In one example, fluorocarbons are considered to have a very low surface tension, because of the unique polarity (electronegativity) of its carbon-fluorine bond. In other examples, the surface tension of a layer of a tightly structured Langmuir-Blodgett type film may be primarily determined by the percent of fluorine in the terminus of the alkyl chains. For such tightly ordered films, a single terminal trifluoromethyl group may render a surface nearly as lipophobic as a perfluoroalkyl layer. Further, fluorocarbons covalently attached to an underlying derivatized solid support (e.g. a highly crosslinked polymeric), exhibit reactive sites densities lower than that of a Langmuir-Blodgett type film. For example, the surface tension of a methyltrimethoxysilane surface is about 22.5 mN/m, and the surface tension of an aminopropyltriethoxysilane surface is about 35 mN/m.

A surface is generally considered to exhibit hydrophilic behavior when its critical surface tension is greater than 45 mN/m. As such, surfaces with highly critical surface tensions, exhibit a small contact angle and stronger adsorptive behavior. A surface is generally considered to exhibit hydrophobic behavior when its critical surface tension is less than 35 mN/m. A low critical surface tension is associated with oleophilic behavior (i.e. the wetting of the surfaces by hydrocarbon oils). Surfaces with a surface tension below 20 mN/m, may resist wetting through hydrocarbon oils and are considered to be both oleophobic and hydrophobic.

Silane surface modification may be used to generate a broad range of critical surface tensions. Devices and methods disclosed herein include surface coatings, e.g. those involving silanes, to achieve surface tensions of less than 5, 6, 7, 8, 9, 10, 12, 15, 20, 25, 30, 35, 40, 45, 50, 60, 70, 80, 90, 100, 110, 115, 120 mN/m, or higher. Further, in some cases, the methods and devices disclosed herein use surface coatings, e.g. those involving silanes, to achieve surface tensions of more than 115, 110, 100, 90, 80, 70, 60, 50, 45, 40, 35, 30, 25, 20, 15, 12, 10, 9, 8, 7, 6 mN/m or less.

The water contact angle and the surface tension of non-limiting examples of surface coatings, e.g., those involving silanes, are described in Table 1 and Table 2 of Arkles et al. (Silanes and Other Coupling Agents, Vol. 5v: The Role of Polarity in the Structure of Silanes Employed in Surface Modification. 2009), which is incorporated herein by reference in its entirety. The tables are replicated below.

TABLE 1
Contact angles of water (degrees) on smooth surfaces

| | |
|---|---|
| Heptadecafluorodecyltrimethoxysilane | 113-115 |
| Poly(tetrafluoroethylene) | 108-112 |
| Polypropylene | 108 |
| Octadecyldimethylchlorosilane | 110 |
| Octadecyltrichlorosilane | 102-109 |
| Tris(trimethylsiloxy)silylethyldimethylchlorosilane | 103-104 |
| Octyldimethylchlorosilane | 104 |
| Butyldimethylchlorosilane | 100 |
| Trimethylchlorosilane | 90-100 |
| Polyethylene | 88-103 |
| Polystyrene | 94 |
| Poly(chlorotrifluoroethylene) | 90 |
| Human skin | 75-90 |
| Diamond | 87 |
| Graphite | 86 |
| Silicon (etched) | 86-88 |
| Talc | 82-90 |
| Chitosan | 80-81 |
| Steel | 70-75 |
| Methoxyethoxyundecyltrichlorosilane | 73-74 |
| Methacryloxypropyltrimethoxysilane | 70 |
| Gold, typical (see gold, clean) | 66 |
| Intestinal mucosa | 50-60 |
| Kaolin | 42-46 |
| Platinum | 40 |
| Silicon nitride | 28-30 |
| Silver iodide | 17 |
| [Methoxy(polyethyleneoxy)propyl]trimethoxysilane | 15-16 |
| Sodalime glass | <15 |
| Gold, clean | <10 |
| Trimethoxysilylpropyl substituted poly(ethyleneimine), | <10 |
| hydrochloride | |

TABLE 2
Critical surface tensions (mN/m)

| | |
|---|---|
| Heptadecafluorodecyltrichlorosilane | 12 |
| Poly(tetrafluoroethylene) | 18.5 |
| Octadecyltrichlorosilane | 20-24 |
| Methyltrimethoxysilane | 22.5 |
| Nonafluorohexyltrimethoxysilane | 23 |
| Vinyltriethoxysilane | 25 |
| Paraffin wax | 25.5 |
| Ethyltrimethoxysilane | 27.0 |
| Propyltrimethoxysilane | 28.5 |
| Glass, sodalime (wet) | 30.0 |
| Poly(chlorotrifluoroethylene) | 31.0 |
| Polypropylene | 31.0 |
| Poly(propylene oxide) | 32 |
| Polyethylene | 33.0 |
| Trifluoropropyltrimethoxysilane | 33.5 |
| 3-(2-Aminoethyl)aminopropyltrimethoxysilane | 33.5 |
| Polystyrene | 34 |
| p-Tolyltrimethoxysilane | 34 |
| Cyanoethyltrimethoxysilane | 34 |
| Aminopropyltriethoxysilane | 35 |
| Acetoxypropyltrimethoxysilane | 37.5 |
| Poly(methyl methacrylate) | 39 |
| Poly(vinyl chloride) | 39 |
| Phenyltrimethoxysilane | 40.0 |
| Chloropropyltrimethoxysilane | 40.5 |
| Mercaptopropyltrimethoxysilane | 41 |
| Glycidoxypropyltrimethoxysilane | 42.5 |
| Poly(ethylene terephthalate) | 43 |
| Copper (dry) | 44 |
| Poly(ethylene oxide) | 43-45 |
| Aluminum (dry) | 45 |
| Nylon 6/6 | 45-46 |
| Iron (dry) | 46 |
| Glass, sodalime (dry) | 47 |
| Titanium oxide (anatase) | 91 |
| Ferric oxide | 107 |
| Tin oxide | 111 |

In some embodiments, the device described herein comprises a surface, wherein one or more regions of the surface comprises a plurality of second molecules, wherein the plurality of second molecules bind to the surface, lacks a reactive group capable of binding to a nucleoside, and are inert to the conditions of ordinary oligonucleotide synthesis (e.g. the solid surface may be devoid of free hydroxyl, amino, or carboxyl groups to the bulk solvent interface during monomer addition, depending on the selected chemistry).

In some embodiments, the surface of the device disclosed herein is layered with one or more different layers of compounds. Such layers of interest may include, without limitation, inorganic and organic layers such as metals, metal oxides, polymers, small organic molecules, and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyetheyleneamines, polyarylene sulfides, polysiloxanes, polyimides, polyacetates, and any other suitable compounds described herein or otherwise known in the art. In some cases, polymers are heteropolymeric. In some cases, polymers are homopolymeric. In some cases, polymers comprise functional moieties or are conjugated.

In some cases, one or more regions of the surface of the device comprises comprise a reactive moiety prior to the start of a first cycle of synthesis, or prior to or during a first number of cycles of an oligonucleotide synthesis process, wherein the reactive moieties may be quickly depleted to unmeasurable densities after one, two, three, four, five, or more cycles of the oligonucleotide synthesis reaction. In some embodiments, the surface is further optimized for well or pore wetting, (e.g., by common organic solvents such as acetonitrile and the glycol ethers or aqueous solvents, relative to surrounding surfaces).

### Methods of Forming Functionalized Surfaces

Disclosed herein are devices for the synthesis of polynucleotides at a low error rate, a low dropout rate, a high yield, and a high oligo representation. In some embodiments, surface characteristics of a coated surface may be adjusted in various ways that are suitable for oligonucleotide synthesis. Devices disclosed herein comprise a surface having varying wettability characteristics and varying ability to couple a nucleoside. In some embodiments, a device disclosed herein comprises a surface with a plurality of active regions, wherein each active region exhibits a high surface energy and comprises a first molecule comprising a high-energy molecule, wherein the first molecule binds to the surface of the device and to a nucleoside, thereby supporting a coupling reaction to the surface. In some embodiments, each of the plurality of the active regions is surrounded by a passive region, wherein the passive region comprises a second molecule which exhibits a low surface energy, wherein the second molecule binds to the surface and lacks a reactive group capable of binding to a nucleoside, thereby preventing a coupling reaction to the surface. In some embodiments, the nucleoside comprises a nucleic acid monomer.

Provided herein is a method for functionalization of a surface of a device comprising: (a) providing a device having a surface that comprises silicon dioxide; and (b) silanizing the surface using, a suitable silanizing agent described herein or otherwise known in the art, for example, an organofunctional alkoxysilane molecule. In some cases, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some cases, a surface comprises functionalization with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene.

To achieve surfaces with low density of nucleoside-coupling agents, a mixture of both active and passive functionalization agents is mixed and deposited at a plurality of predetermined regions of the surface of a device disclosed herein. In some embodiments, the mixture of active and passive functionalization agents provides for regions of the surface of the device having less than a saturating amount of the active functionalization agent, therefore lowering the density of the functionalization agent in particular regions. A mixture of agents that bind to a surface disclosed herein may be deposited on predetermined regions of the surface, wherein the coated surface provides for a density of synthesized polynucleotides that is about 10%, 20%, 30%, 40%, 50%, 60%, 70%, or 80% less than the density of synthesized polynucleotides extended from a region of the surface comprising only the active functionalization agent. The mixture may comprise (i) a silane that binds the surface and couples to a nucleoside and (ii) a silane that binds to the surface and does not couple a nucleoside that is deposited on the predetermined region of the surface of the device disclosed herein, wherein coated surface of the device provides for a density of synthesized polynucleotides that is about 10%, 20%, 30%, 40%, 50%, 60%, 70%, or 80% less than the density of synthesized polynucleotides extended from a region of the surface comprising (i) the silane that binds the surface and couples to a nucleoside and not (ii) the silane that binds the surface and does not couple a nucleoside. A predetermined region of a surface may be treated with a diluted amount of an active functionalization agent disclosed herein to reduce the density of the synthesized polynucleotides by about 50% compared to an identical surface coated with a non-diluted amount of the active functionalization agents.

One exemplary active functionalization agent that may optionally be included in a mixture disclosed herein is 3-glycidoxypropyltrimethoxysilane. For example, the mixture may include 3-glycidoxypropyltrimethoxysilane or propyltrimethoxysilane; or 3-glycidoxypropyltrimethoxysilane and propyltrimethoxysilane.

Regions surrounding those regions deposited with the mixture may be coated with a passive functionalization agent having a lower surface energy. In some cases, the passive functionalization agent is a fluorosilane. Exemplary fluorosilanes include (tridecafluoro-1,1,2,2-tetrahydrooctyl)trichlorosilane, perfluorooctyltrichlorosilane, perfluorooctyltriethoxysilane, and perfluorooctyltrimethoxychloro silane.

More broadly, an active functionalization agent may comprise a silane, such as an aminosilane. In some cases, the active functionalization agent comprises a silane that, once activated, couples to a nucleoside, e.g., a nucleoside phosphoramidite. The active functionalization agent may be a silane that has a higher surface energy than the passive functionalization agent deposited on areas of the surface located outside of predetermined regions where the silane is deposited. In some cases, both molecules types in the mixture comprise silanes, wherein the mixture is deposited on the surface. In one example, one of the molecules in the mixture is a silane that binds the surface and couples to a nucleoside, and another molecule in the mixture is a silane that binds to the surface and does not couple a nucleoside. In such cases, both molecules in the mixture, when deposited on the surface, provide for a region having a higher surface energy than surrounding regions.

Agents in a mixture disclosed here are chosen from suitable reactive and inert moieties, which dilute the surface density of reactive groups to a desired level for downstream reactions, where both molecules have a similar surface energy. In some cases, the density of the portion of a surface functional group that reacts to form a growing oligonucleotide in an oligonucleotide synthesis reaction is about 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 7.0, 10.0, 15.0, 20.0, 50.0, 75.0, or about 100.0 μmol/m².

Mixtures disclosed herein may comprise at least 2, 3, 4, 5, or more different types of functionalization agents. A mixture may comprise 1, 2, 3, or more silanes. The ratio between the at least two types of surface functionalization agents in a mixture deposited on a surface disclosed herein may range from about 1:1 to 1:100 with the active functionalization agent being diluted to a greater amount compared to a functionalization agent that does not couple a nucleoside. In some cases, the ratio of the at least two types of surface functionalization agents in a mixture deposited on a surface disclosed herein is about 1:100 to about 1:2500, with the active functionalization agent being diluted to a greater amount compared to a functionalization agent that does not couple a nucleoside.

Exemplary ratios of the at least two types of surface functionalization agents in a mixture deposited on a surface disclosed herein include at least 1:10, 1:50, 1:100, 1:200, 1:500, 1:1000, 1:2000, 1:2500, 1:3000, or 1:5000, with the active functionalization agent being diluted to a greater amount compared to a functionalization agent that does not couple a nucleoside. An exemplary specific ratio between the at least two types of surface functionalization agents in a mixture is about 1:2000, with the active functionalization agent being diluted to a greater amount, compared to a functionalization agent that does not couple a nucleoside.

Another exemplary specific ratio between the at least two types of surface functionalization agents in a mixture is 1:2000, wherein the active functionalization agent is diluted to a greater amount, compared to a functionalization agent that does not couple a nucleoside.

The passive functionalization agent deposited on the surface may be a fluorosilane molecule. Exemplary fluorosilane molecules are (tridecafluoro-1,1,2,2-tetrahydrooctyl)trichlorosilane and perfluorooctyltrichlorosilane. The mixture deposited on a surfaced disclosed herein may comprise a silane that binds the surface and nucleoside phosphoramidite and is diluted about 1:100 to about 1:2500 with a silane that binds to the surface and does not bind to a nucleoside phosphoramidite. In some cases, the silane molecule deposited on a surfaced disclosed herein is diluted about 1:2000.

To attain a reduction in active agent density at particular locations on a surface of the device disclosed herein, deposition at regions for nucleic acid extension with the non-nucleoside molecule of the mixture occurs prior to deposition of the mixture itself. In some case, the mixture deposited on a surfaced disclosed herein comprises 3-glycidoxypropyltrimethoxysilane diluted at a ratio of about 1:2000. The mixture deposited on a surfaced disclosed herein may comprise 3-glycidoxypropyltrimethoxysilane diluted at a ratio of about 1:2000 in propyltrimethoxysilane. In one example, a surface disclosed herein is first deposited at regions for nucleic acid extension with propyltrimethoxysilane prior to deposition of a mixture of propyltrimethoxysilane and 3-glycidoxypropyltrimethoxysilane. In some cases, a silane deposited at sites of polynucleotide synthesis is selected from the group consisting of 11-acetoxyundecyltriethoxysilane, n-decyltriethoxysilane, (3-aminopropyl)trimethoxysilane, (3-aminopropyl)triethoxysilane, 3-glycidyloxypropyl/trimethoxysilane and N-(3-triethoxysilylpropyl)-4-hydroxybutyramide or a portion of the surface, functionalized or modified to be more hydrophilic or hydrophobic as compared to the surface or the portion of the surface prior to the functionalization or modification.

Provided herein is a device comprising a surface have a predetermined first region comprising two or more molecules, wherein each of the two or more molecules have a different ability to couple a nucleoside and have a similar water contact angle. In some cases, the difference in the water contact angle between the two or more molecules is less than 40°, 35°, 30°, 25°, 20°, 15° or 10° as measured on one or more smooth or planar equivalent surfaces. In some cases, the first region is surrounded by a second region, where the first and second region have a difference in water contact angle of greater than 90°, 85°, 80°, 75°, 70°, 65°, 60°, 55°, 50°, 45°, 40°, 35°, 30°, 25°, 20°, 15° or 10° as measured on one or more smooth or planar equivalent surfaces. In some cases, the first region is surrounded by a second region, wherein the first and the second region have a difference in water contact angle of at least 90°, 85°, 80°, 75°, 70°, 65°, 60°, 55°, 50°, 45°, 40°, 35°, 30°, 25°, 20°, 15° or 10° as measured on one or more smooth or planar equivalent surfaces. Unless otherwise stated, water contact angles mentioned herein correspond to measurements taken on uncurved, smooth, or planar equivalents of the surfaces in question.

### Methods of De Novo Polynucleotide Synthesis

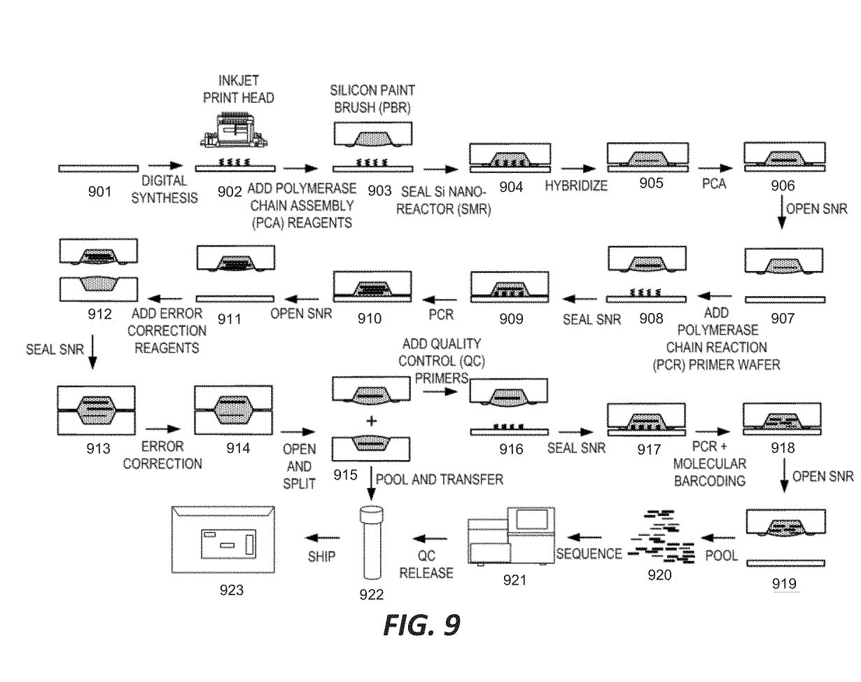

Devices having modified surfaces described herein may be used for de novo synthesis processes. An exemplary workflow for one such process is divided generally into phases: (1) de novo synthesis of a single stranded polynucleotide library, (2) joining polynucleotides to form larger fragments, (3) error correction, (4) quality control, and (5) shipment, FIG. 9. Prior to de novo synthesis, an intended nucleic acid sequence or group of nucleic acid sequences is preselected. For example, a group of genes is preselected for generation.

Once preselected nucleic acids for generation are selected, a predetermined library of polynucleotides is designed for de novo synthesis. Various suitable methods are known for generating high density polynucleotide arrays. In the workflow example, a surface layer 901 is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells that increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A device, such as an polynucleotide synthesizer (a material deposition device), is designed to release reagents in a step wise fashion such that multiple polynucleotides extend, in parallel, one residue at a time to generate oligomers with a predetermined nucleic acid sequence 902. In some cases, polynucleotides are cleaved from the surface at this stage. Cleavage may include gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 903. Prior to or after the sealing 904 of the polynucleotides, a reagent is added to release the polynucleotides from the surface. In the exemplary workflow, the polynucleotides are released subsequent to sealing of the nanoreactor 905. Once released, fragments of single stranded polynucleotides hybridize in order to span an entire long-range sequence of DNA. Partial hybridization 905 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 906.

After PCA is complete, the nanoreactor is separated from the surface 907 and positioned for interaction with a polymerase 908. After sealing, the nanoreactor is subject to PCR 909 and the larger nucleic acids are formed. After PCR 910, the nanochamber is opened 911, error correction reagents are added 912, the chamber is sealed 913 and an error correction reaction occurs to remove mismatched base pairs and/or strands with poor complementarity from the double stranded PCR amplification products 914. The nanoreactor is opened and separated 915. Error corrected product is next subject to additional processing steps, such as PCR and molecular bar coding, and then packaged 922 for shipment 923.

In some cases, quality control measures are taken. After error correction, quality control steps include for example interaction with a wafer having sequencing primers for amplification of the error corrected product 916, sealing the wafer to a chamber containing error corrected amplification product 917, and performing an additional round of amplification 918. The nanoreactor is opened 919 and the products are pooled 920 and sequenced 921. After an acceptable quality control determination is made, the packaged product 922 is approved for shipment 923.

The devices described herein comprise actively functionalized surfaces configured to support the attachment and synthesis of polynucleotides. Synthesized polynucleotides include polynucleotides comprising modified and/or non-canonical bases and/or modified backbones. In various methods, a library of polynucleotides having pre-selected sequences is synthesized on a device disclosed herein. In some cases, one or more of the polynucleotides has a different sequence and/or length than another polynucleotide in the library. The stoichiometry of each polynucleotide synthesized on a surface is controlled and tunable by varying one or more features of the surface and/or polynucleotide sequence to be synthesized; one or more methods for surface functionalization and/or polynucleotide synthesis; or a combination thereof. In some instances, controlling the density of a growing polynucleotide on a resolved locus of a device disclosed herein allows for polynucleotides to be synthesized with a low error rate.

Provided herein are devices comprising a surface that supports the synthesis of a plurality of polynucleotides having different predetermined sequences at addressable locations on a common support. The surface of a device disclosed herein may support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides.

In some case, at least a portion of the polynucleotides have an identical sequence or are configured to be synthesized with an identical sequence. Devices disclosed herein provides for a surface environment for the growth of polynucleotides having at least about 10, 20, 30, 50, 60, 70, 75, 80, 85, 90, 95, 100, 110, 120, 130, 140, 150, 160, 175, 200, 225, 250, 275, 300, 325, 350, 375, 400, 425, 450, 475, 500, 1000, 2000 bases or more in length. Provided herein are devices comprising a surface that supports the synthesis of at least about 10, 20, 30, 50, 60, 70, 75, 80, 90, 100, 120, 150, 200, 300, 400, 500, 600, 700, 800, or more bases.

A library of polynucleotides may be synthesized, wherein a population of distinct polynucleotides are assembled to generate a larger nucleic acid comprising at least about 500; 1,000; 2,000; 3,000; 4,000; 5,000; 6,000; 7,000; 8,000; 9,000; 10,000; 11,000; 12,000; 13,000; 14,000; 15,000; 16,000; 17,000; 18,000; 19,000; 20,000; 25,000; 30,000; 40,000; or 50,000 bases. Polynucleotide synthesis methods described herein are useful for the generation of an polynucleotide library comprising at least 500; 1,000; 5,000; 10,000; 20,000; 50,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 2,200,000; 2,400,000; 2,600,000; 2,800,000; 3,000,000; 3,500,000; 4,00,000; or 5,000,000 distinct polynucleotides. In some case, at least about 1 pmol, 10 pmol, 20 pmol, 30 pmol, 40 pmol, 50 pmol, 60 pmol, 70 pmol, 80 pmol, 90 pmol, 100 pmol, 150 pmol, 200 pmol, 300 pmol, 400 pmol, 500 pmol, 600 pmol, 700 pmol, 800 pmol, 900 pmol, 1 nmol, 5 nmol, 10 nmol, 100 nmol or more of an polynucleotide is synthesized within a locus.

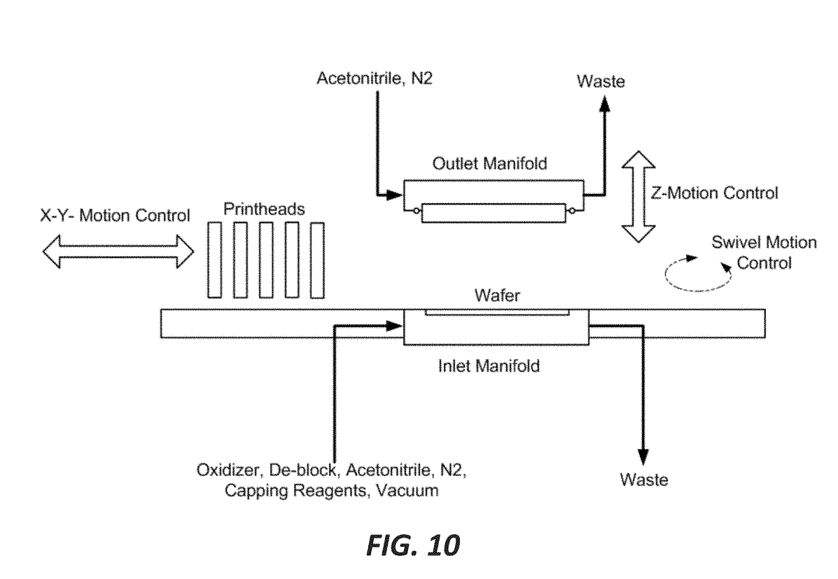

Polynucleotides are synthesized on a surface described herein using a system comprising a polynucleotide synthesizer material deposition device that deposits reagents necessary for synthesis, FIG. 10. Reagents for polynucleotide synthesis include, for example, reagents for polynucleotide extension and wash buffers. As non-limiting examples, the polynucleotide synthesizer deposits coupling reagents, capping reagents, oxidizers, de-blocking agents, acetonitrile and gases such as nitrogen gas. In addition, the polynucleotide synthesizer optionally deposits reagents for the preparation and/or maintenance of device integrity. The polynucleotide synthesizer comprises material deposition devices that may move in the X-Y direction to align with the location of the surface of the device. The polynucleotide synthesizer may also move in the Z direction to seal with the surface of the device, forming a resolved reactor.

Methods are provided herein where at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 100000 or more nucleic acids may be synthesized in parallel. Total molar mass of nucleic acids synthesized within the device or the molar mass of each of the nucleic acids may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more.

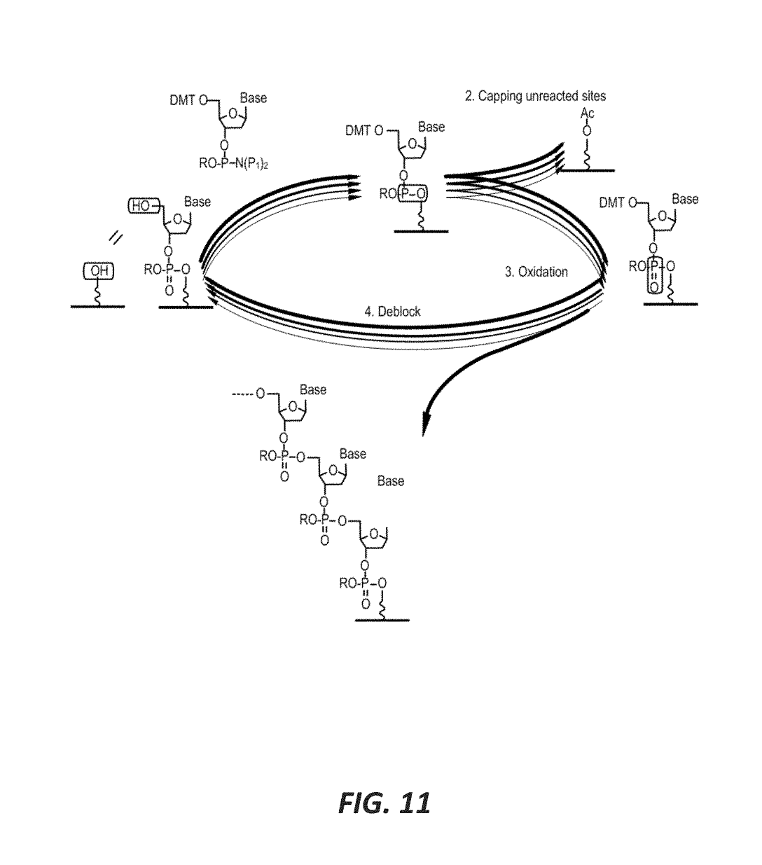

Polynucleotide synthesis methods disclosed herein include enzyme independent methods. An example of a synthesis method that is useful with the devices provided herein is one that incorporates phosphoramidite chemistry, FIG. 11. Typically, after the deposition of a monomer, e.g., a mononucleotide, a dinucleotide, or a longer oligonucleotide with suitable modifications for phosphoramidite chemistry one or more of the following steps may be performed at least once to achieve the step-wise synthesis of high-quality polymers in situ: 1) Coupling, 2) Capping, 3) Oxidation, 4) Sulfurization, and 5) Deblocking (detritylation). Washing steps typically intervenes steps 1 to 5.

Provided herein are methods wherein a polynucleotide error rate is dependent on the efficiency of one or more chemical steps of polynucleotide synthesis. In some cases, polynucleotide synthesis comprises a phosphoramidite method, wherein a base of a growing polynucleotide chain is coupled to phosphoramidite. Coupling efficiency of the base is related to the error rate. For example, higher coupling efficiency correlates to lower error rates. In some cases, the devices and/or synthesis methods described herein allow for a coupling efficiency greater than 98%, 98.5%, 99%, 99.5%, 99.6%, 99.7%, 99.8%, 99.9%, 99.95%, 99.96%, 99.97%, 99.98%, or 99.99%. In some cases, a polynucleotide synthesis method comprises a double coupling process, wherein a base of a growing polynucleotide chain is coupled with a phosphoramidite, the polynucleotide is washed and dried, and then treated a second time with a phosphoramidite. Efficiency of deblocking in a phosphoramidite polynucleotide synthesis method also contributes to error rate. In some cases, the devices and/or synthesis methods described herein allow for removal of 5′-hydroxyl-protecting groups at efficiencies greater than 98%, 98.5%, 99%, 99.5%, 99.6%, 99.7%, 99.8%, 99.9%, 99.95%, 99.96%, 99.97%, 99.98%, or 99.99%. Error rate may be reduced by minimization of depurination side reactions.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it may react with a subsequently applied protected monomer; and application of another protected monomer for linking. One or more intermediate steps include oxidation or sulfurization. In some cases, one or more wash steps precede or follow one or all of the steps.

Methods disclosed herein provide for at least 20,000 or more non-identical polynucleotides each at least 30 bases in length are synthesized, wherein each of the at least 20,000 non-identical polynucleotides extends from a different locus of the patterned surface. Methods disclosed herein provides for at least 20,000 non-identical polynucleotides collectively encoding for at least 200 preselected nucleic acids, and having an aggregate error rate of less than 1 in 1000 bases compared to predetermined sequences without correcting errors. Methods disclosed herein provide for at least 6,000 or more non-identical polynucleotides each at least 30 bases in length are synthesized, wherein each of the at least 6,000 non-identical polynucleotides extends from a different locus of the patterned surface. Methods disclosed herein provide for at least 6,000 non-identical polynucleotides collectively encoding for at least 50 preselected nucleic acids, and having an aggregate error rate of less than 1 in 1000 bases compared to predetermined sequences without correcting errors. Methods disclosed herein provide for at least 100,000 or more non-identical polynucleotides each at least 30 bases in length are synthesized, wherein each of the at least 100,000 non-identical polynucleotides extends from a different locus of the patterned surface. Methods disclosed herein provide for at least 100,000 non-identical polynucleotides collectively encoding for at least 750 preselected nucleic acids, and having an aggregate error rate of less than 1 in 1000 bases compared to predetermined sequences without correcting errors. In some instances, the aggregate error rate is less than 1 in 1500, less than 1 in 2000 bases, less than 1 in 3000 bases or less compared to the predetermined sequences. Surfaces provided herein provide for the low error rates.

Provided herein are systems polynucleotide synthesis, comprising: a material deposition device comprising plurality of reagents for polynucleotide synthesis and a plurality of nozzles for depositing the plurality of reagents for polynucleotide synthesis; a computer for controlling the release of the plurality of reagents for polynucleotide synthesis from the plurality of nozzles; and a plate disclosed herein for receiving the plurality of reagents for polynucleotide synthesis.

Oligonucleotide Libraries with Low Error Rates

The term “error rate” may also be referred to herein as a comparison of the collective sequence encoded by polynucleotides generated compared to the sequence of one or more predetermined longer nucleic acid, e.g., a gene. An aggregate “error rate” refers to the collective error rate of synthesized nucleic acids compared to the predetermined sequences for which the nucleic acids are intended to encode. Error rates include mismatch error rate, deletion error rate, insertion error rate, insertion/deletion error rate, any combination thereof. Methods and devices herein provide for low error rates are for synthesized polynucleotide libraries having at least 20,000, 40,000, 60,000, 80,000, 100,000, 200,000, 300,000, 400,000, 500,000, 600,000, 700,000, 800,000, 1,000,000, or 2,000,000 or more polynucleotides. Loci may be configured to comprise a population of polynucleotides, wherein the population may be configured to comprise polynucleotides having the same or different sequences.

Devices and methods described herein provide for a low overall error rate for the individual types of errors are achieved. Individual types of error rates include deletions, insertions, or substitutions for a polynucleotide library synthesized. In some cases, polynucleotides synthesized have an aggregate error rate of about 1:500, 1:1000, 1:1500, 1:2000, 1:3000, 1:4000, 1:5000, 1:6000, 1:7000, 1:8000, 1:9000, 1:10000 or less. These error rates may be for at least 50%, 60%, 70%, 80%, 90%, 95%, 98%, 99%, 99.5%, or more of the polynucleotides synthesized.

Methods described herein provide synthesis of polynucleotides having an average deletion error rate of about 1:500, 1:1000, 1:1500, 1:1700, 1:2000, 1:3000, 1:4000, 1:5000, 1:6000, 1:7000, 1:8000, 1:9000, 1:10000 or less. Methods described herein provide synthesis of polynucleotides having an aggregate deletion error rate of about 1:500, 1:1000, 1:1500, 1:1700, 1:2000, 1:3000, 1:4000, 1:5000, 1:6000, 1:7000, 1:8000, 1:9000, 1:10000 or less. Methods described herein provide synthesis of polynucleotides having an aggregate insertion error rate of about 1:500, 1:1000, 1:1500, 1:2000, 1:3000, 1:4000, 1:5000, 1:6000, 1:7000, 1:8000, 1:9000, 1:10000 or less. Methods described herein provide synthesis of polynucleotides having an aggregate insertion error rate of about 1:500, 1:1000, 1:1500, 1:2000, 1:3000, 1:4000, 1:5000, 1:6000, 1:7000, 1:8000, 1:9000, 1:10000 or less. Methods described herein provide synthesis of polynucleotides having an aggregate substitution error rate of about 1:500, 1:1000, 1:1500, 1:2000, 1:3000, 1:4000, 1:5000, 1:6000, 1:7000, 1:8000, 1:9000, 1:10000 or less. Methods described herein provide synthesis of polynucleotides having an aggregate insertion error rate of about 1:500, 1:1000, 1:1500, 1:2000, 1:3000, 1:4000, 1:5000, 1:6000, 1:7000, 1:8000, 1:9000, 1:10000 or less. Methods described herein provide synthesis of polynucleotides having an aggregate substitution error rate of about 1:500, 1:1000, 1:1500, 1:2000, 1:3000, 1:4000, 1:5000, 1:6000, 1:7000, 1:8000, 1:9000, 1:10000 or less. The overall error rate or error rates for individual types of errors such as deletions, insertions, or substitutions for each polynucleotide synthesized, may be for at least 50%, 60%, 70%, 80%, 90%, 95%, 98%, 99%, 99.5%, or more of the oligonucleotides synthesized.

In some cases, where a particular nucleobase is frequently observed as being associated with an increase in error rate, e.g., due to deletion or insertion error rate, a predetermined sequence is designed to have the error-prone nucleobase replaced with another base such that the replacement(s) would not change the codons for which the sequence encodes. In some cases, where a particular nucleobase is observed to frequently be associated with a decrease in error rate, e.g., due to deletion or insertion error rate, a predetermined sequence is designed to have one or more other error prone nucleobases replaced with less-error-prone nucleobase such that the replacement(s) would not change the codons for which the sequence encodes.

### Polynucleotide Release and Assembly

Polynucleotides synthesized using the methods and devices described herein, are optionally released from the surface from which they were synthesized. In some cases, polynucleotides are cleaved from the surface at this stage. Cleavage may include gaseous cleavage, e.g., with gaseous ammonia or gaseous methylamine. Loci in a single cluster collectively correspond to sequence encoding for a single gene, and, when cleaved, may remain on the surface of the loci within a cluster. The application of ammonia gas is used to simultaneously deprotect phosphates groups protected during the synthesis steps, i.e. removal of electron-withdrawing cyano group. Once released from the surface, polynucleotides may be assembled into larger nucleic acids. Synthesized polynucleotides are useful, for example, as components for gene assembly/synthesis, site-directed mutagenesis, nucleic acid amplification, microarrays, and sequencing libraries.

Provided herein are methods where polynucleotides of predetermined sequence are designed to collectively span a large region of a target sequence, such as a gene. In some cases, larger polynucleotides are generated through ligation reactions to join the synthesized polynucleotides. One example of a ligation reaction is polymerase chain assembly (PCA). In some cases, at least of a portion of the polynucleotides are designed to include an appended region that is a substrate for universal primer binding. For PCA reactions, the presynthesized polynucleotides include overlaps with each other (e.g., 4, 20, 40, or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double stranded DNA. In some cases, after the PCA reaction is complete, an error correction step is conducted using mismatch repair detecting enzymes to remove mismatches in the sequence. Once larger fragments of a target sequence are generated, they may be amplified. For example, in some cases, a target sequence comprising 5′ and 3′ terminal adaptor sequences is amplified in a polymerase chain reaction (PCR) which includes modified primers, e.g., uracil containing primers the hybridize to the adaptor sequences.

Provided herein are methods wherein following polynucleotide synthesis, polynucleotides within one cluster are released from their respective surfaces and pooled into the common area, such as a well. In some cases, the pooled polynucleotides are assembled into a larger nucleic acid, such as a gene, within the well. In some cases, at least about 1, 10, 50, 100, 200, 240, 500, 1000, 10000, 20000, 50000, 100000, 1000000, or more nucleic acids are assembled from polynucleotides synthesized on a surface of a device disclosed herein. In some instances, each assembled nucleic acid comprises a gene. In some instances, each assembled nucleic acid comprises a vector or plasmid sequence.

A pass-printing scheme may be used to deliver reagents to loci in a cluster, as wells as to transfer synthesis reaction products to another location. At least 2, 3, 4, 5, 6, 7, 8, 9, or 10 passes may be used to deliver reagents. In some cases, assembled nucleic acids generated by methods described herein have a low error rate compared to a predetermined sequence without correcting errors. In some cases, assembled nucleic acids generated by methods described herein have an error rate of less than 1:1000, 1:1500, 1:2000, 1:2500, 1:3000 bases compared to a predetermined sequence without correcting errors.

### Alignment Marks

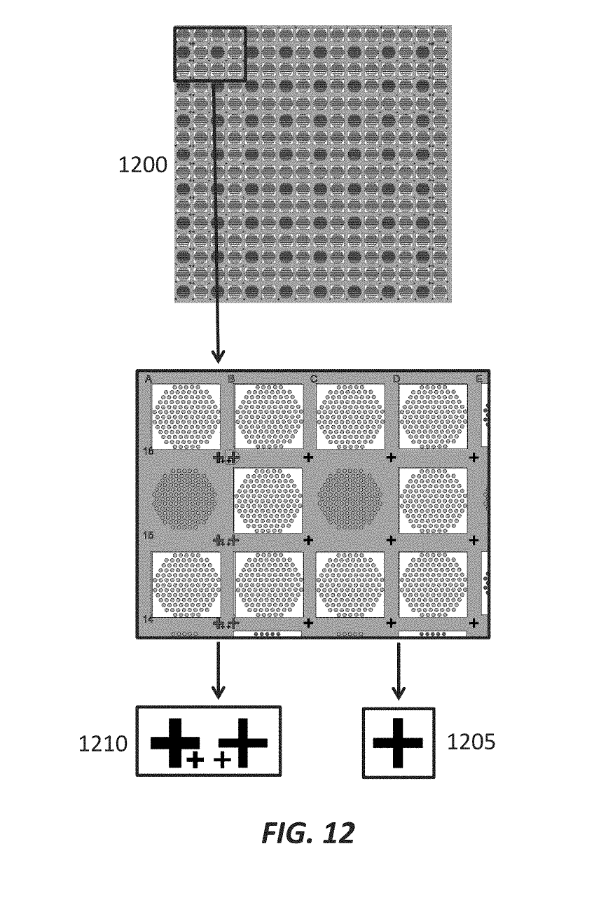

During the deposition process, references points on a surface are used by a machine for calibration purposes. Surfaces described herein may comprise fiducial marks, global alignment marks, lithography alignment marks, or a combination thereof. Fiducial marks are generally placed on the surface of a device, such as an array of clusters 1200 to facilitate alignment of such devices with other components of a system; FIG. 12 illustrates an exemplary arrangement. The surface of a device disclosed herein may have one or more fiducial marks, e.g. 2, 3, 4, 5, 6, 7, 8, 12, 10. In some cases, fiducial marks may are used for global alignment of the microfluidic device.

Fiducial marks may have various shapes and sizes. In some cases, a fiducial mark has the shape of a square, circle, triangle, cross, “×”, addition or plus sign, subtraction or minus sign, or any combination thereof. In some one example, a fiducial mark is in the shape of an addition or a plus sign 1205. In some cases, a fiducial mark comprises a plurality of symbols. Exemplary fiducial mark may comprise one or more plus signs 1210, e.g., 2, 3, 4, or more plus signs. In one example, a fiducial mark comprises 4 plus signs.

Fiducial marks may be located on the surface of devices disclosed herein. A fiducial mark may be about 0.5 μm, 1 μm, 2 μm, 3 μm, 4 μm, 5 μm, 6 μm, 7 μm, 8 μm, 9 μm, 10 μm, 100 μm, 1000 μm, 2000 μm, 5000 μm, 7000 μm, 8000 μm, 9000 μm, or 10,000 μm, from the center of the surface. In some cases, the fiducial mark is located from about 0.1 mm to about 10 mm from the edge of the surface portion, e.g., about 0.5 mm from the edge. In some case, the fiducial is located from about 1 mm to about 10 mm form a cluster, e.g., 1.69 mm. In some instances, a distance from the center of a fiducial mark and a nearest corner of a surface in one dimension is from about 0.5 mm to about 10 mm, e.g., about 1 mm. In some instances, a length of a fiducial mark in one dimension is from about 0.5 mm to about 5 mm, e.g., about 1 mm. In some instances, the width of a fiducial mark is from about 0.01 mm to about 2 mm, e.g., 0.05 mm.

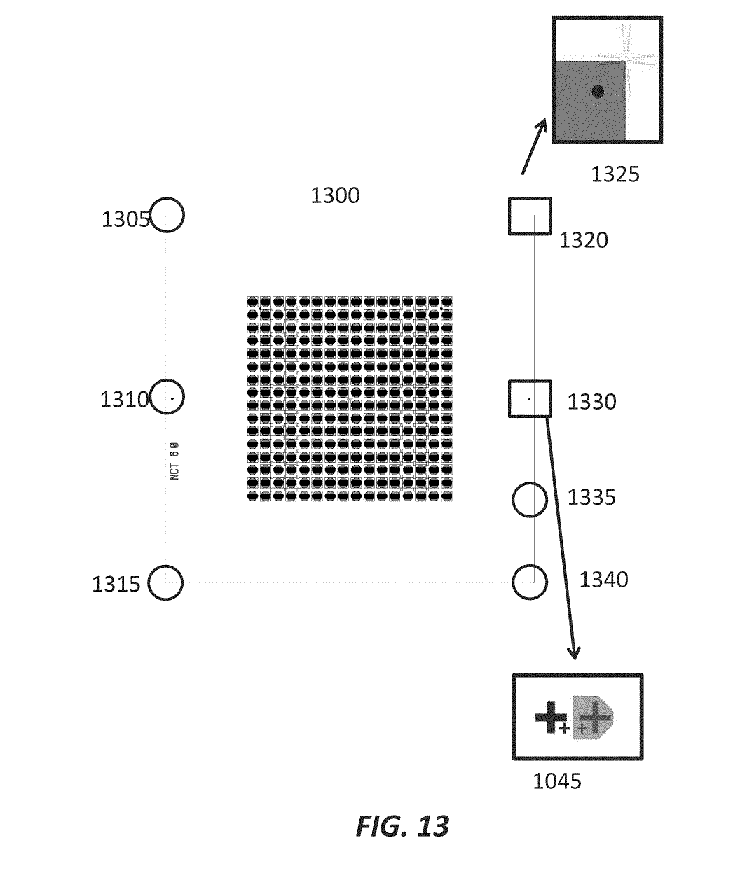

Global alignment marks may have various shapes and sizes. Global alignment marks are placed on the surface of a device described herein to facilitate alignment of such devices with other components of a system; FIG. 13 illustrates an exemplary arrangement. Exemplary global alignment marks have the shape of a square, circle, triangle, cross, “×”, addition or plus sign, subtraction or minus sign, or any combination thereof. Exemplary global alignment marks include the shape of a circle 1325 or a plus mark 1345. In some cases, a global alignment mark is located near an edge of the substrate portion, as shown by the location of marks 1305, 1310, 1315, 1320, 1330, 1335, and 1340. A global alignment mark may comprise a plurality of symbols. In some case, a global alignment mark comprises one or more circles, e.g., 2, 3, 4, or more plus signs.

A global alignment mark may be located on the surface of a device disclosed herein. In some cases, the global alignment mark is about 0.5 μm, 1 μm, 2 μm, 3 μm, 4 μm, 5 μm, 6 μm, 7 μm, 8 μm, 9 μm, 10 μm, 100 μm, 1000 μm, 2000 μm, 5000 μm, 7000 μm, 8000 μm, 9000 μm, or 10,000 μm, from the center of the surface. In some case, the global alignment mark is about 0.5 μm, 1 μm, 2 μm, 3 μm, 4 μm, 5 μm, 6 μm, 7 μm, 8 μm, 9 μm, 10 μm, 100 μm, 1000 μm,2000 μm, 5000 μm, 7000 μm, 8000 μm, 9000 μm, or 10,000 μm, from the edge of the surface. In some cases, the global alignment mark is about 0.5 μm, 1 μm, 2 μm, 3 μm, 4 μm, 5 μm, 6 μm, 7 μm, 8 μm, 9 μm, 10 μm, 100 μm, 200 μm, 300 μm, 400 μm, 500 μm, 750 μm, or 1000 μm in size. In an example arrangement, the global alignment mark is about 125 μm in diameter and is located about 1000 μm from the edge of the surface of the device. Surfaces of a device disclosed herein may comprise one or more global alignment marks, e.g. 2, 3, 4, 5, 6, 7, 8, 9, 10, or more marks. The distance between the global alignment marks may be about 1 μm, 2 μm, 3 μm, 4 μm, 5 μm, 6 μm, 7 μm, 8 μm, 9 μm, 10 μm, 100 μm, 500 μm, 1000 μm, 2000 μm, 5000 μm, 7000 μm, 8000 μm, 9000 μm, or 10,000 μm.

### Computer Systems

Methods are provided herein for attachment of pre-synthesized oligonucleotide and/or polynucleotide sequences to a support and in situ synthesis of the same using light-directed methods, flow channel and spotting methods, inkjet methods, pin-based methods and/or bead-based methods are used. In some cases, pre-synthesized oligonucleotides are attached to a support or are synthesized using a spotting methodology wherein monomers solutions are deposited drop wise by a dispenser that moves from region to region. In one example, oligonucleotides are spotted on a support using a mechanical wave actuated dispenser.

The systems described herein may further include a member for providing a droplet to a first spot (or feature) having a plurality of support-bound oligonucleotides. The droplet may include one or more compositions comprising nucleotides or oligonucleotides (also referred herein as nucleotide addition constructs) having a specific or predetermined nucleotide to be added and/or reagents that allow one or more of hybridizing, denaturing, chain extension reaction, ligation, and digestion. In some cases, different compositions or different nucleotide addition constructs may be deposited at different addresses on the support during any iteration so as to generate an array of predetermined oligonucleotide sequences (the different features of the support having different predetermined oligonucleotide sequences). One particularly useful way of depositing the compositions is by depositing one or more droplet, each droplet containing the desired reagent (e.g. nucleotide addition construct) from a pulse jet device spaced apart from the support surface, onto the support surface or features built into the support surface.

A substrate with resolved features is “addressable” when it has multiple regions of different moieties (e.g., different polynucleotide sequences) such that a region (i.e., a “feature” or “spot” of the substrate) at a particular predetermined location (i.e., an “address”) on the substrate will detect a particular target or class of targets (although a feature may incidentally detect non-targets of that location). Substrate features are typically, but need not be, separated by intervening spaces. In some cases, features may be built into a substrate and may create one-, two-, or three-dimensional microfluidic geometries. A “substrate layout” refers to one or more characteristics of the features, such as feature positioning on the substrate, one or more feature dimensions, and an indication of a molecule at a given location.

Any of the systems described herein, may be operably linked to a computer, and may be automated through a computer either locally or remotely. Methods and disclosed herein may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. The computer systems may be programmed to interface between the user specified base sequence and the position of a material deposition device to deliver the correct reagents to specified regions of the substrate.

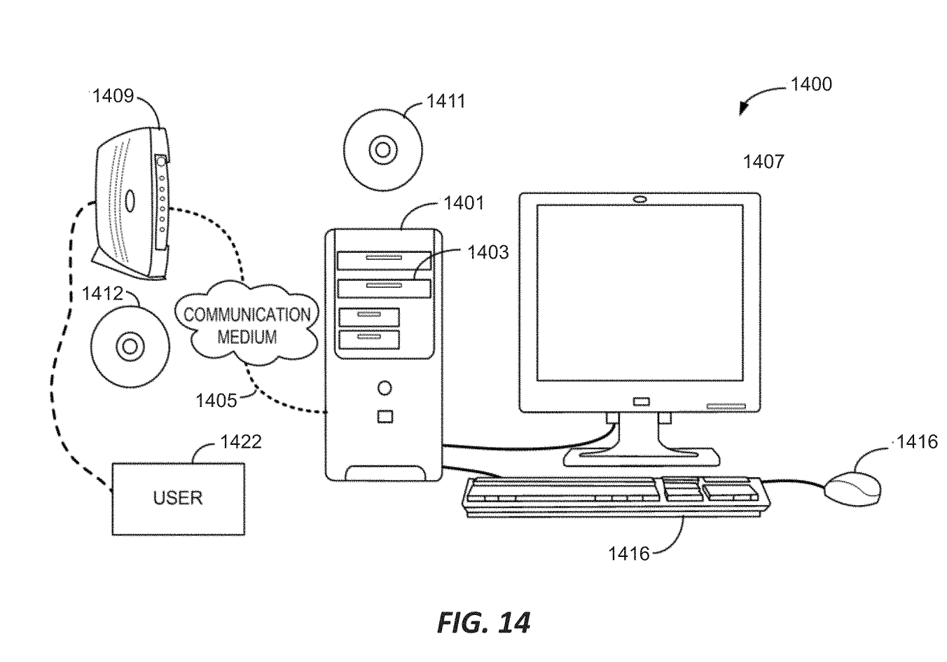

The computer system 1400 illustrated in FIG. 14 may be understood as a logical apparatus that may read instructions from media 1411 and/or a network port 1405, which may optionally be connected to server 1409 having fixed media 1412. The system may include a CPU 1401, disk drives 1403, optional input devices such as keyboard 1415 and/or mouse 1416 and optional monitor 1407. Data communication may be achieved through the indicated communication medium to a server at a local or a remote location. The communication medium may include any means of transmitting and/or receiving data. For example, the communication medium may be a network connection, a wireless connection, or an internet connection. Such a connection may provide for communication over the World Wide Web. It is envisioned that data relating to the present disclosure may be transmitted over such networks or connections for reception and/or review by a party 1422.

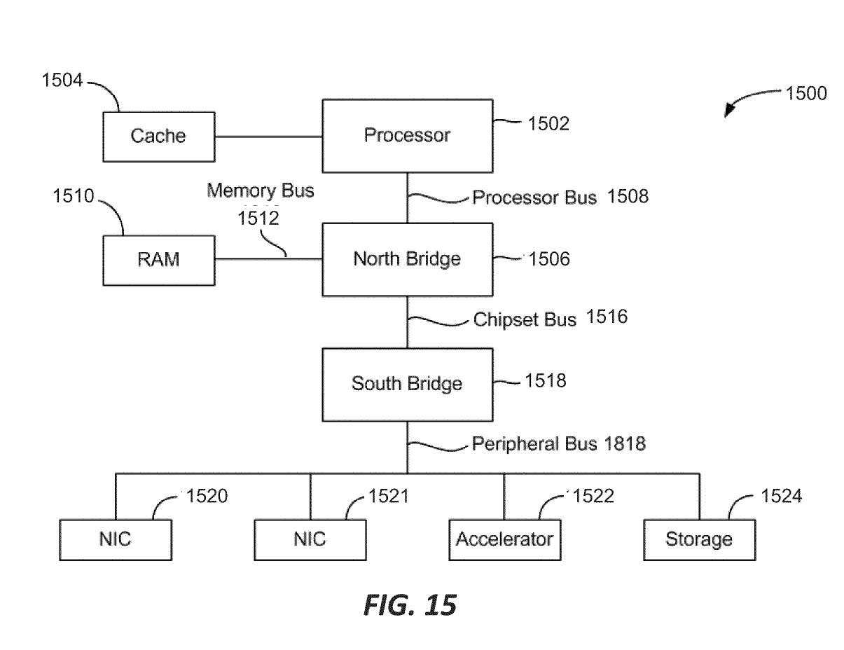

FIG. 15 is a block diagram illustrating a first example architecture of a computer system 1500 that may be used in connection with example embodiments of the present invention. An example computer system may include a processor 1502 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally equivalent processor. Multiple threads of execution may be used for parallel processing. In some embodiments, multiple processors or processors with multiple cores may also be used, whether in a single computer system, in a cluster, or distributed across systems over a network comprising a plurality of computers, cell phones, and/or personal data assistant devices.

A high-speed cache 1504 may be connected to, or incorporated in, the processor 1502 to provide a high-speed memory for instructions or data that have been recently, or are frequently, used by processor 1502. The processor 1502 is connected to a north bridge 1506 by a processor bus 1508. The north bridge 1506 is connected to random access memory (RAM) 1510 by a memory bus 1512 and manages access to the RAM 1510 by the processor 1502. The north bridge 1506 is also connected to a south bridge 1514 by a chipset bus 1516. The south bridge 1514 is, in turn, connected to a peripheral bus 1518. The peripheral bus may be, for example, PCI, PCI-X, PCI Express, or other peripheral bus. The north bridge and south bridge are often referred to as a processor chipset and manage data transfer between the processor, RAM, and peripheral components on the peripheral bus 1518. In some alternative architectures, the functionality of the north bridge may be incorporated into the processor instead of using a separate north bridge chip. In some embodiments, system 1500 may include an accelerator card 2022 attached to the peripheral bus 1518. The accelerator may include field programmable gate arrays (FPGAs) or other hardware for accelerating certain processing. For example, an accelerator may be used for adaptive data restructuring or to evaluate algebraic expressions used in extended set processing.

Software and data are stored in external storage 1524 and may be loaded into RAM 1510 and/or cache 1504 for use by the processor. The system 1500 includes an operating system for managing system resources; non-limiting examples of operating systems include: Linux, Windows™, MACOS™, BlackBerry OS™, iOS™, and other functionally equivalent operating systems, as well as application software running on top of the operating system for managing data storage and optimization in accordance with example embodiments of the present invention. In this example, system 1500 also includes network interface cards (NICs) 1520 and 1521 connected to the peripheral bus for providing network interfaces to external storage, such as Network Attached Storage (NAS) and other computer systems that may be used for distributed parallel processing.

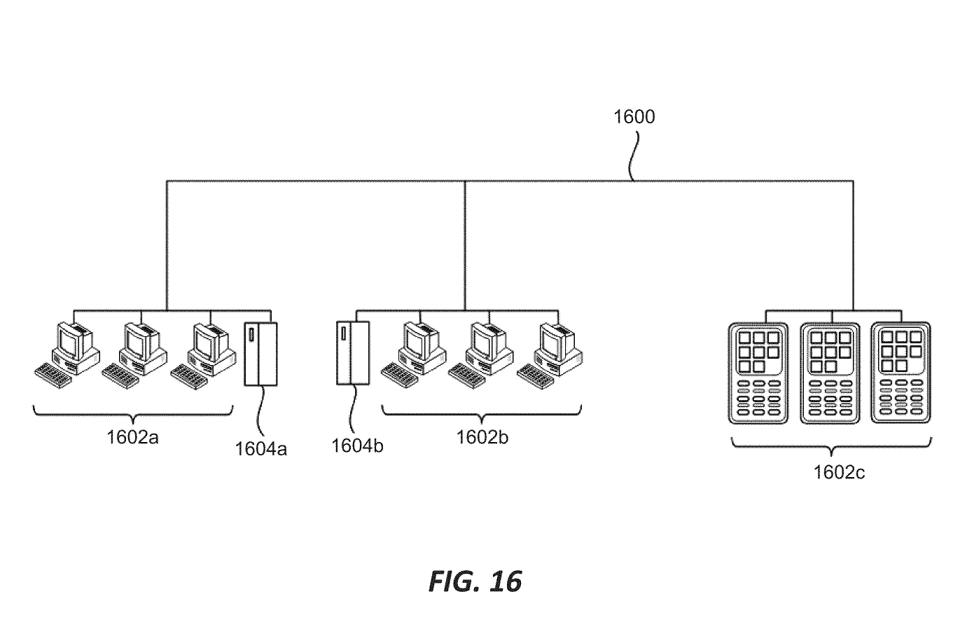

FIG. 16 is a diagram showing a network 1600 with a plurality of computer systems 1602a, and 1602b, a plurality of cell phones and personal data assistants 1602c, and Network Attached Storage (NAS) 1604a, and 1604b. In example embodiments, systems 1602a, 1602b, and 1602c may manage data storage and optimize data access for data stored in Network Attached Storage (NAS) 1604a and 1604b. A mathematical model may be used for the data and be evaluated using distributed parallel processing across computer systems 1602a, and 1602b, and cell phone and personal data assistant systems 1602c. Computer systems 1602a, and 1602b, and cell phone and personal data assistant systems 1602c may also provide parallel processing for adaptive data restructuring of the data stored in Network Attached Storage (NAS) 1604a and 1604b. FIG. 16 illustrates an example only, and a wide variety of other computer architectures and systems may be used in conjunction with the various embodiments of the present invention. For example, a blade server may be used to provide parallel processing. Processor blades may be connected through a back plane to provide parallel processing. Storage may also be connected to the back plane or as Network Attached Storage (NAS) through a separate network interface. In some cases, processors may maintain separate memory spaces and transmit data through network interfaces, back plane, or other connectors for parallel processing by other processors. In other embodiments, some or all of the processors may use a shared virtual address memory space.

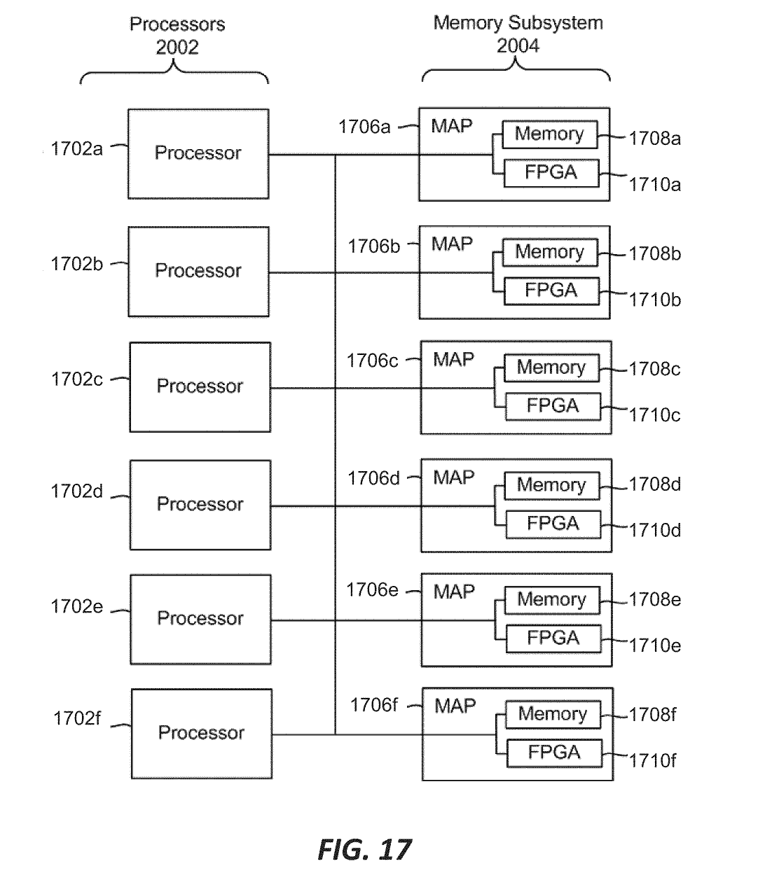

FIG. 17 is a block diagram of a multiprocessor computer system 1700 using a shared virtual address memory space in accordance with an example embodiment. The system includes a plurality of processors 1702a-f that may access a shared memory subsystem 1704. The system incorporates a plurality of programmable hardware memory algorithm processors (MAPs) 1706a-f in the memory subsystem 1704. Each MAP 1706a-f may comprise a memory 1708a-f and one or more field programmable gate arrays (FPGAs) 1710a-f. The MAP provides a configurable functional unit and particular algorithms or portions of algorithms may be provided to the FPGAs 1710a-f for processing in close coordination with a respective processor. For example, the MAPs may be used to evaluate algebraic expressions regarding the data model and to perform adaptive data restructuring in example embodiments. In this example, each MAP is globally accessible by all of the processors for these purposes. In one configuration, each MAP may use Direct Memory Access (DMA) to access an associated memory 1708a-f, allowing it to execute tasks independently of, and asynchronously from, the respective microprocessor 1702a-f. In this configuration, a MAP may feed results directly to another MAP for pipelining and parallel execution of algorithms.

Software and data are stored in external storage 1724 and may be loaded into RAM 1710 and/or cache 1704 for use by the processor. The system 1700 includes an operating system for managing system resources; non-limiting examples of operating systems include: Linux, Windows™, MACOS™, BlackBerry OS™, iOS™, and other functionally equivalent operating systems, as well as application software running on top of the operating system for managing data storage and optimization in accordance with example embodiments of the present invention. In this example, system 1700 also includes network interface cards (NICs) 1720 and 1721 connected to the peripheral bus for providing network interfaces to external storage, such as Network Attached Storage (NAS) and other computer systems that may be used for distributed parallel processing.

The above computer architectures and systems are examples only, and a wide variety of other computer, cell phone, and personal data assistant architectures and systems may be used in connection with example embodiments, including systems using any combination of general processors, co-processors, FPGAs and other programmable logic devices, system on chips (SOCs), application specific integrated circuits (ASICs), and other processing and logic elements. In some cases, all or part of the computer system may be implemented in software or hardware. Any variety of data storage media may be used in connection with example embodiments, including random access memory, hard drives, flash memory, tape drives, disk arrays, Network Attached Storage (NAS) and other local or distributed data storage devices and systems.

The computer system may be implemented using software modules executing on any of the above or other computer architectures and systems. In other examples, the functions of the system may be implemented partially or completely in firmware, programmable logic devices such as field programmable gate arrays (FPGAs), system on chips (SOCs), application specific integrated circuits (ASICs), or other processing and logic elements. For example, the Set Processor and Optimizer may be implemented with hardware acceleration through the use of a hardware accelerator card, such as accelerator card.

### EXAMPLES

The following examples are set forth to illustrate more clearly the principles and practices of embodiments disclosed herein to those skilled in the art, and are not to be construed as limiting the scope of any claimed embodiments. Unless otherwise stated, all parts and percentages are on a weight basis.

### Example 1

### Textured Silicon Arrays

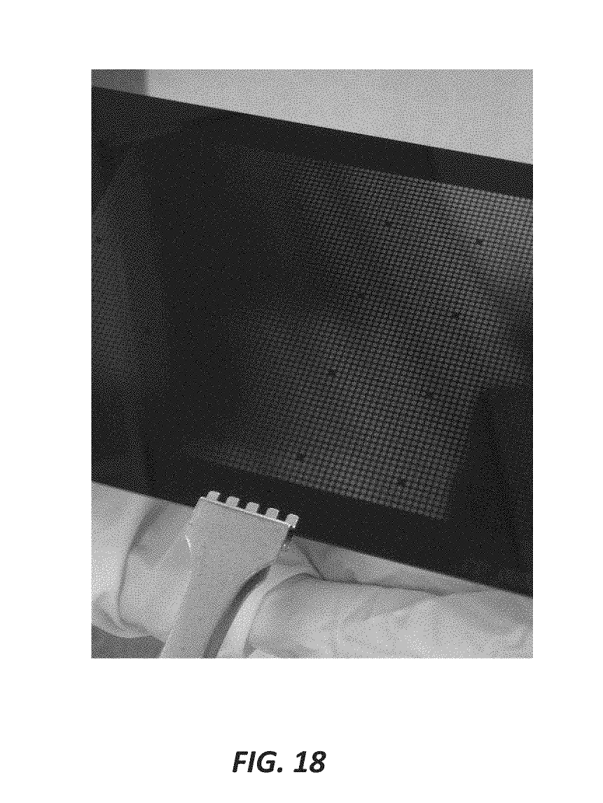

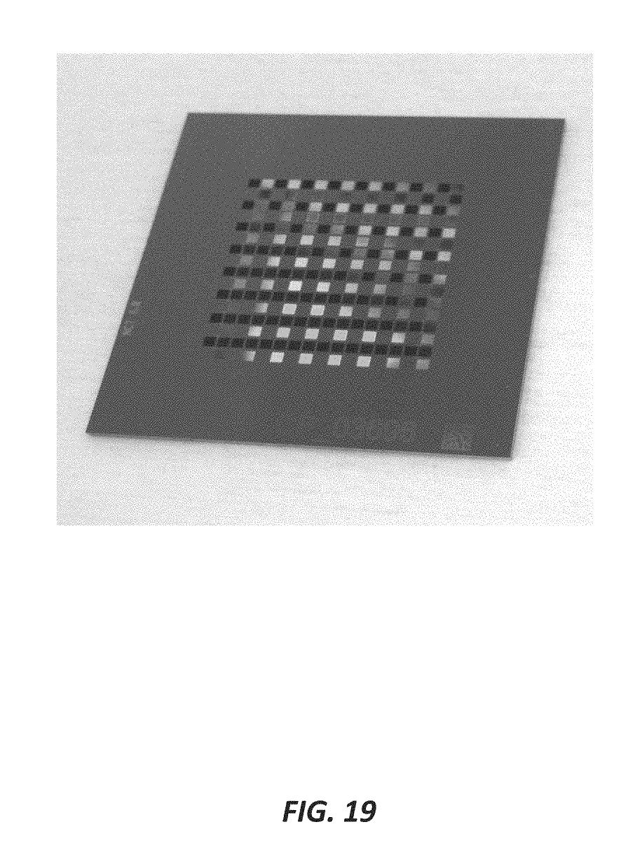

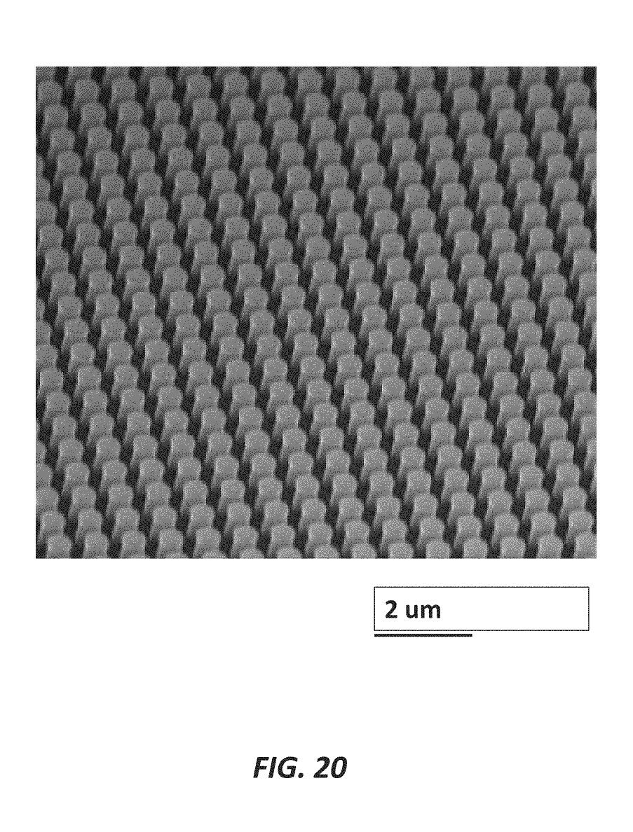

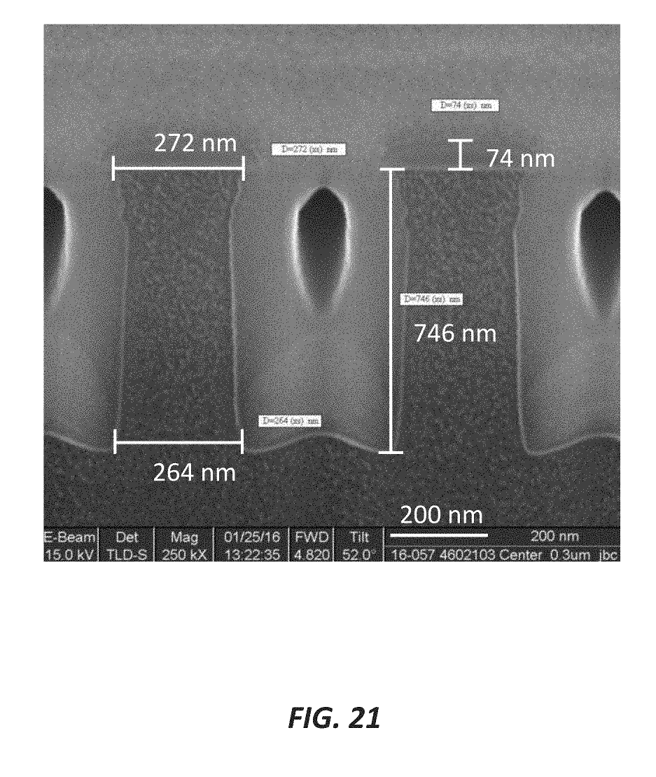

An array of posts was etched into a silicon dioxide wafer to increase surface area by a factor of 2 to 3. Images of the microfluidic device are shown in FIGS. 18-21. FIGS. 18 and 19 are top view image of the microfluidic device. FIG. 20 is a side-view image at 52 degrees of the microfluidic device at a scale to be able to distinguish the posts of the textured surface. FIG. 21 is a side-view the textured microfluidic device.

A textured silicon arrays was manufactured having the following features: etched posts measured 746 nm in height, 272 nm wide at the top of the post, and 264 nm wide at the bottom of the post, and a layer of thermal oxide “caps” the posts was s 74 nm thick.

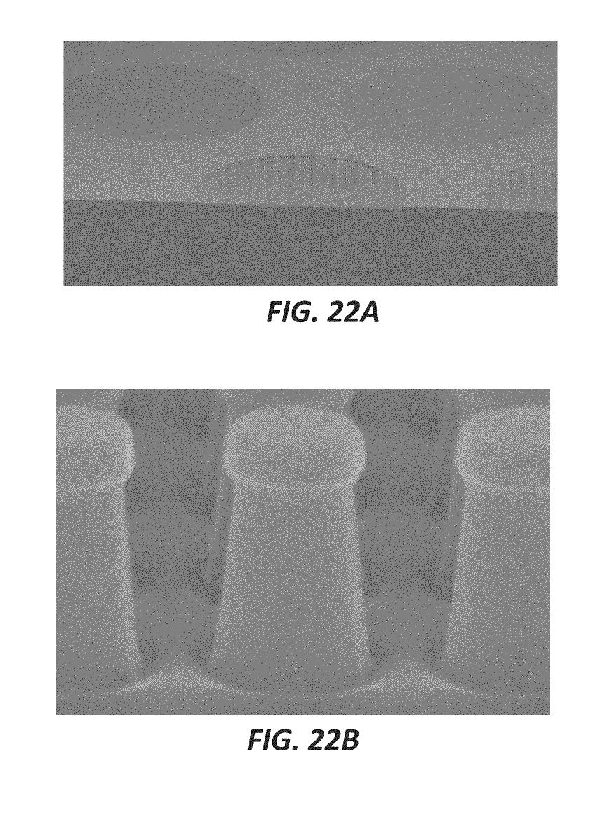

Additional images of the microfluidic device are shown in FIGS. 22A-22B. FIG. 22A is a side view image of the microfluidic device which highlights the macro geometry of loci. FIG. 22B is a side-view image of the microfluidic device at a scale to be able to distinguish the posts of the textured surface. In this example, a wafer contains one chip that measures 140 mm in length 90 mm, wherein the wafer contains a 96×64 array of 6,144 gene clusters and wherein horizontal and vertical pitch of each cluster is 1.125 mm. Further, each cluster contains 121 loci per cluster, yielding 743,424 individually addressable oligo sites per chip.

In a second batch of textured silicon arrays, etched posts measured 746 nm in height, 272 nm wide at the top of the post, and 264 nm wide at the bottom of the post. A layer of thermal oxide “caps” the posts, serving as a mask for the silicon etch, and this layer of thermal oxide is 74 nm thick.

A second textured silicon array was generated etched to have posts with the following features: etched posts measured 304 nm in height, 263 nm wide at the top of the post, and 308 nm wide at the bottom of the post, and a layer of thermal oxide “caps” the posts was s 151 nm in height.

A third textured silicon array was generated etched to have posts with the following features: etched posts measured 432 nm in height, 266 nm wide at the top of the post, and 325 nm wide at the bottom of the post, and a layer of thermal oxide “caps” the posts was s 141 nm in height.

A fourth textured silicon array was generated etched to have posts with the following features: etched posts measured 519 nm in height, 277 nm wide at the top of the post, and 33 nm wide at the bottom of the post, and a layer of thermal oxide “caps” the posts was s 123 nm in height.

### Example 2

### Design, Manufacturing and Analysis of Arrays Having Textured Loci for Polynucleotide Extension

Array design. Silicon plates were manufactured with the following features designed for etched posts: Array A: oxide “caps” on top of the posts of 122 nm in height; etch depth of post of 301 nm; and post width at the base of 320 nm; and Array B: oxide “caps” on top of the posts of 112 nm in height; etch depth of post of 426 nm; and post width at the base of 316 nm.

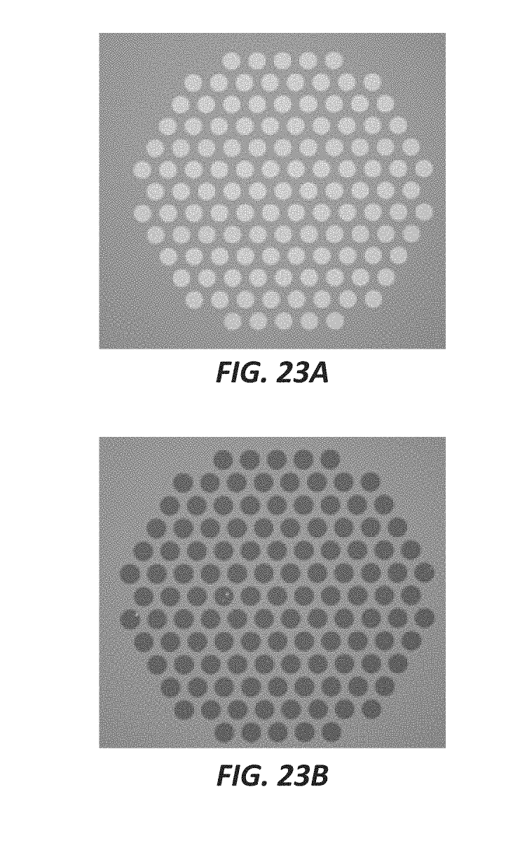

Synthesis on manufactured plates with textured loci. Each of the silicon plates contained an array of clusters, each cluster having discrete locations (“loci”) for nucleotide extension. FIGS. 23A-23B depict low magnification images of a cluster of loci after performing an polynucleotide synthesis reaction. FIG. 23A depicts an image of a cluster of untextured loci. FIG. 23B depicts an image of a cluster of textured loci.

A silicon plate was manufactured with clusters of loci having three different conditions: untextured, “outer” textured or “inner” textured. Each cluster contained 121 loci for polynucleotide synthesis. Methods for chemical synthesis were similar to those described in Example 4. Polynucleotides synthesized were with about 80 to about 100 bases in length.

FIGS. 3A and 3B depicts illustrations of a cluster of inner-textured loci, and an inner-textured locus, respectively. In such an arrangement, the nucleoside coupling reagent was deposited on the surface to a reagent extending beyond the etched textured region. The surface of the device comprised a textured portion 330, an active portion 320, and a passive portion 340. Each of the loci were in the shape of a circle and the total diameter of each loci, the nucleoside reagent coupling deposition region, was 60 um. The diameter of the textured region within each of the loci was also in the shape of a circle and was 55 um. Thus, a distance between the perimeter of the textured region and the perimeter of the loci was 2.5 um. Methods of surface preparation were similar to those described in Example 4.

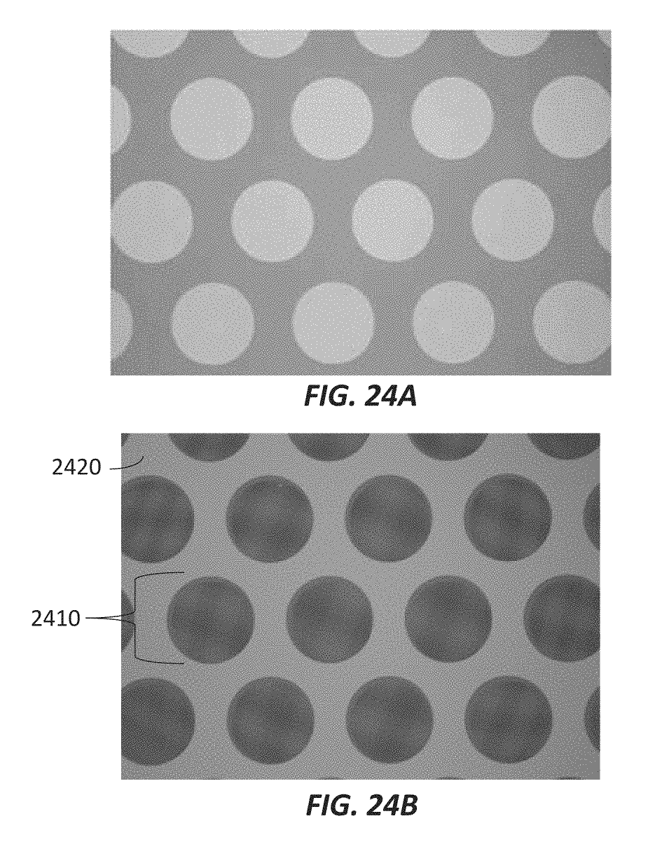

FIGS. 24A-24B depict high magnification images of a cluster of untextured and inner-textured loci, respectively, after polynucleotide synthesis. FIG. 24B depicts an image of a cluster of a textured loci wherein the textured loci 2410 comprises a passive region 2420, an active region without texture 2412 and an active region with texture 2414, the active region being coated with a reagent for nucleoside coupling and the passive region being coated with a reagent that lacks a reactive group for nucleoside coupling.

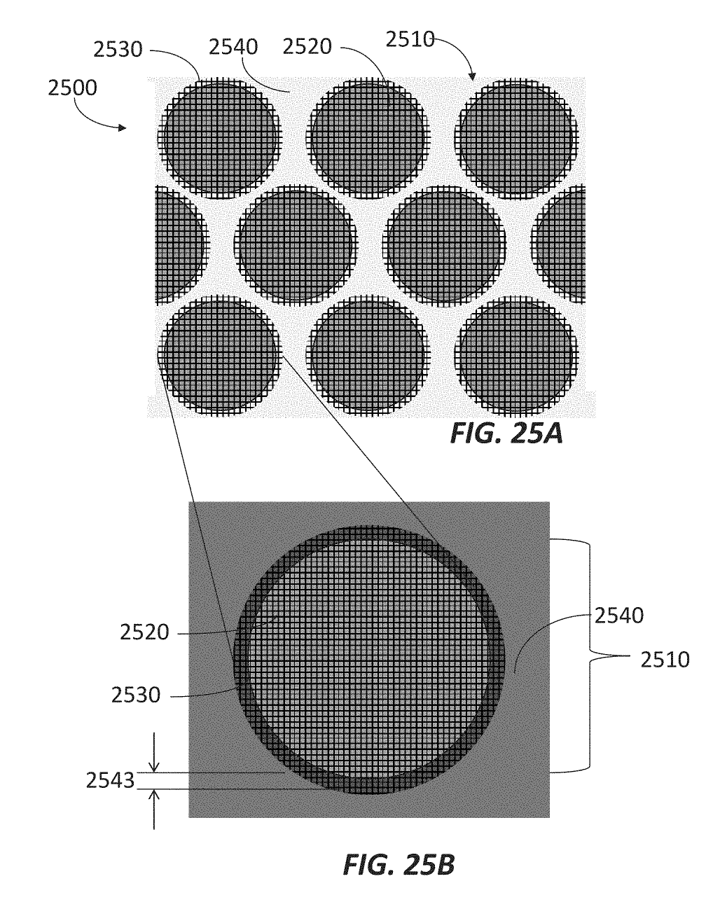

FIGS. 25A and 25B depicts illustrations of a cluster of outer-textured loci, and an outer-textured locus, respectively, wherein the boundary of the textured portion of an outer-textured loci lies entirely outside the boundary of a locus coated with nucleoside coupling reagent. The surface of the device comprises a textured portion 2530, an active portion 2520, and a passive portion 2540. Each of the loci were in the shape of a circle and the total diameter of each loci, the nucleoside reagent coupling deposition region, was 50 um. The diameter of the textured region spanning and extending beyond each of the loci was also in the shape of a circle and was 55 um. Thus, a distance between the perimeter of the textured region and the perimeter of the inner nucleoside coupling loci was 2.5 um.

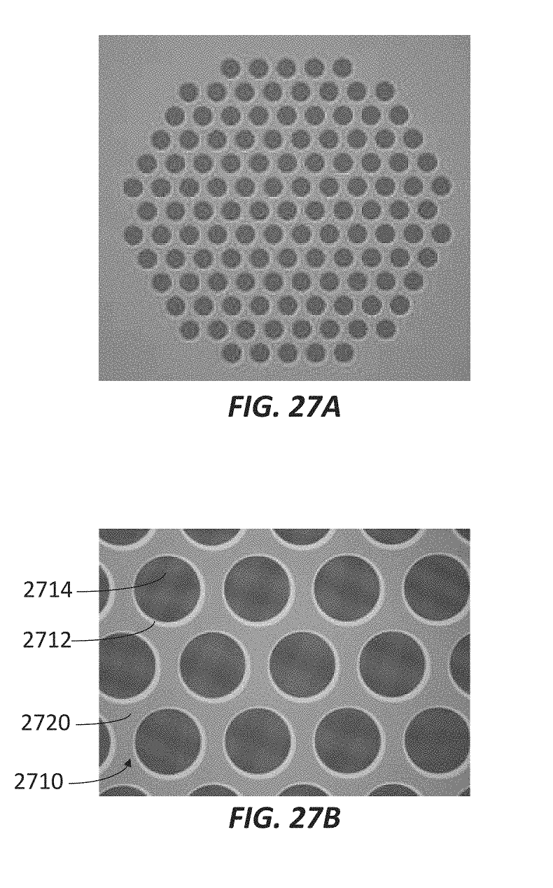

FIGS. 27A-27B depict images of an outer-textured loci after polynucleotide synthesis. FIG. 27A depicts an image of a cluster of outer-textured loci 2710. FIG. 27B depicts a high-magnification image of a outer-textured loci 2710 wherein outer-textured loci 2710 comprises a passive region 2720, an active region without texture 2712 and an active region with texture 2714, the active region being coated with a reagent for nucleoside coupling and the passive region being coated with a reagent that lacks a reactive group for nucleoside coupling.

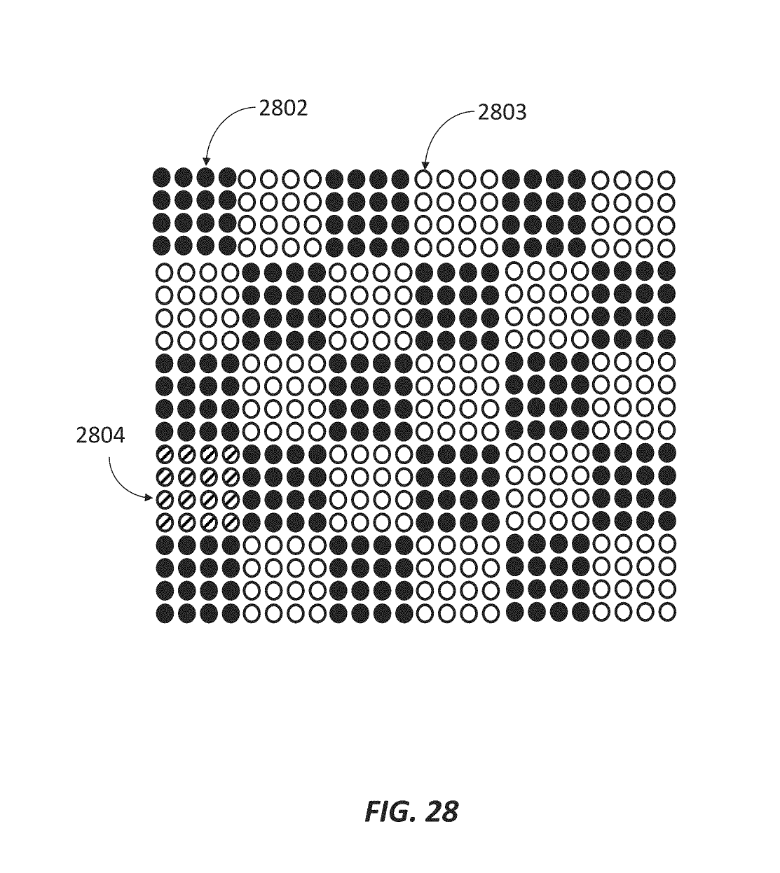

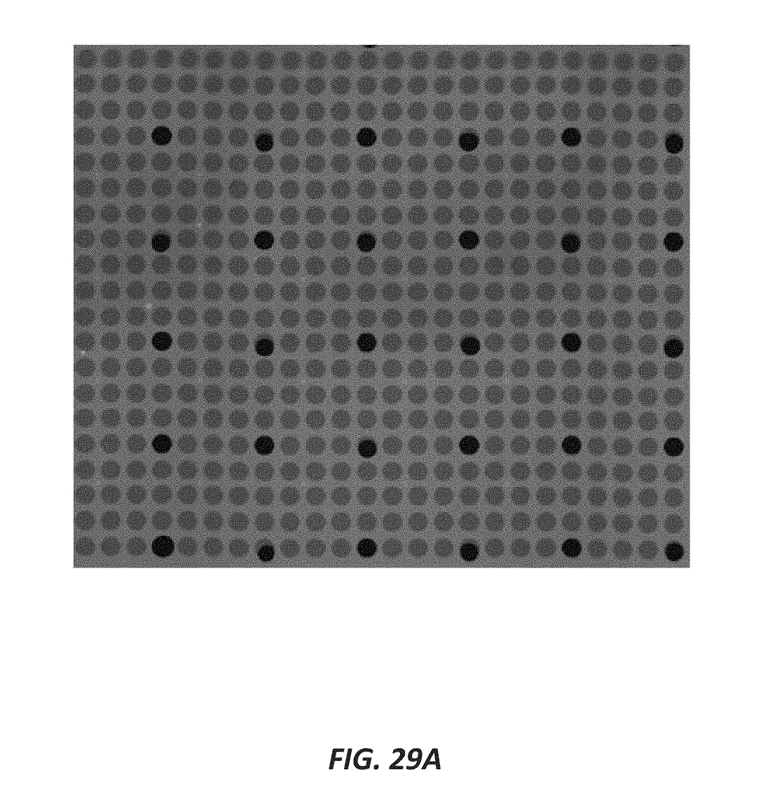

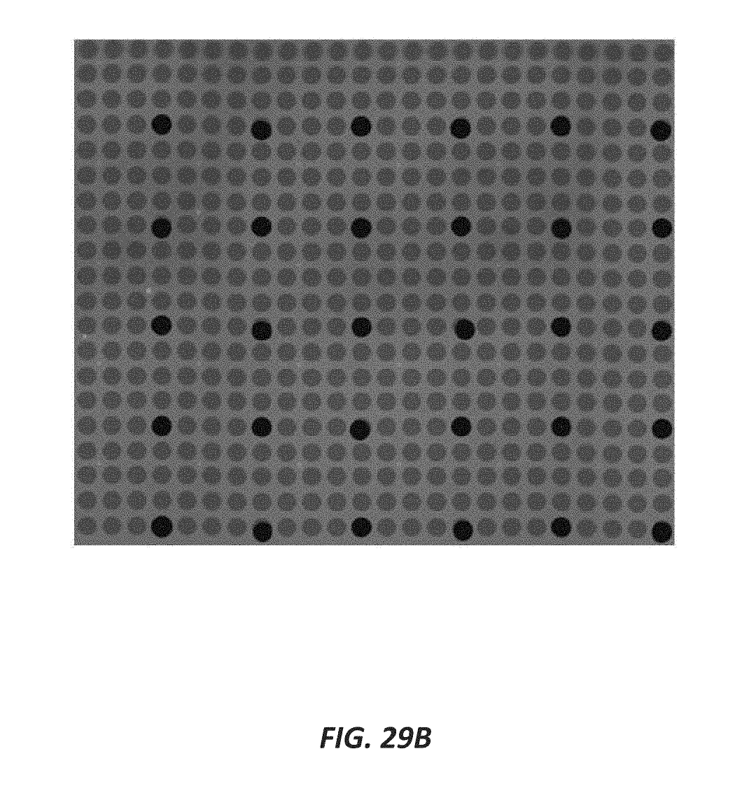

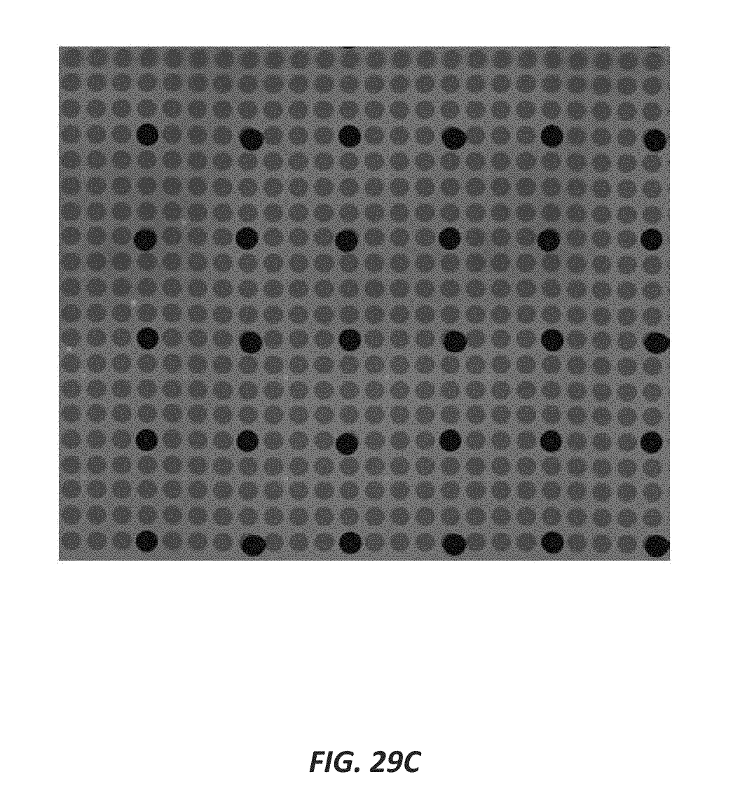

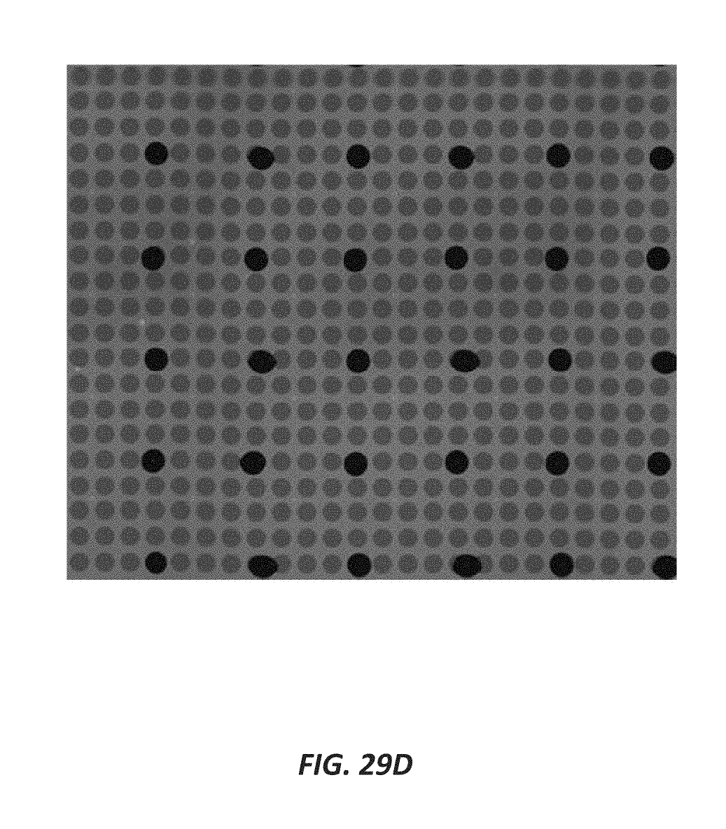

A plate having a checkerboard array was designed as illustrated in FIG. 28: groups of 16 clusters of outer-textured loci 2803, groups of clusters of 16 inner-textured loci 2802, and a group of 16 control clusters of non-textured loci 2804. An image capture following polynucleotide synthesis and deposition of an extraction droplet was taken applying varying droplet volumes. Droplets are indicated by the dark spot in the right corner of each grouping. FIGS. 29A-29D depicts images of droplets on the surfaces, wherein the volume of the droplet is equal to 200 nL, 275 nL, 350 nL, and 425 nl, respectively.

The “inner” design, with nucleoside coupling reagent extending beyond the textured region showed improved alignment of droplets on clusters compared to the “outer” alignment. The variance in droplet volume did not notably impact droplet alignment.

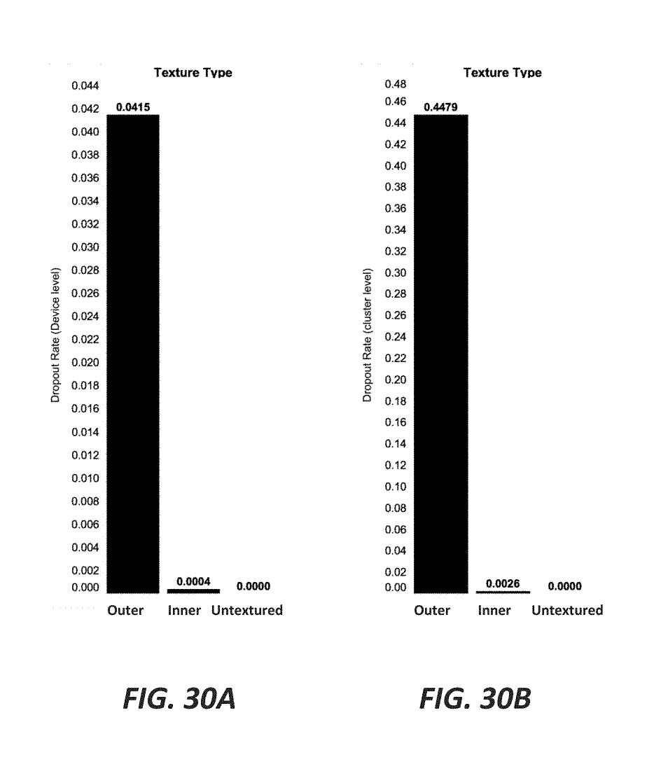

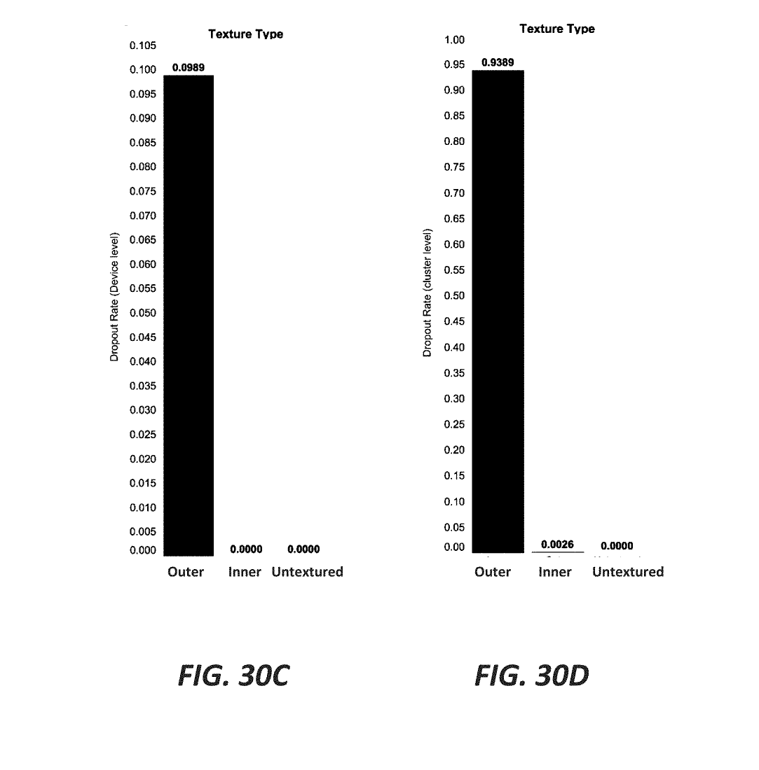

Drop Out Rate. FIGS. 30A-30D depict dropout rates (i.e., frequency of a polynucleotide intended to by synthesized not being detected after sequencing analysis) for two exemplary devices comprising inner-textured loci, outer-textured loci and non-textured loci. For FIGS. 30A-30B, the etch depth was 304 nm and texture width was about 320 nm, for FIGS. 30C-30D, the etch depth was 426 nm and texture width was about 316 nm. FIG. 30A depicts the dropout rates of inner-textured loci, outer-textured loci and non-textured loci on average per a locus. FIG. 30B depicts the dropout rates of inner-textured loci, outer-textured loci and non-textured loci per cluster of loci for a first device. FIG. 30C depicts the dropout rates of inner-textured loci, outer-textured loci and non-textured loci per a locus after a second run on a similar device. FIG. 30D depicts the dropout rates of inner-textured loci, outer-textured loci and non-textured loci per cluster of loci after a second run on a similar device.

Per FIGS. 30A and 30B, inner-textured loci displayed a much lower dropout rate than outer-textured loci, when normalized per device and per cluster, respectively.

Oligo Yield. Average base counts per loci across untextured loci, outer-textured loci and inner-textured loci were measured, as summarized in Table 3. Inner-textured loci demonstrated increased base yield compared to untextured and outer-textured conditions.

TABLE 3

| | | | | Measured Base | |
|---|---|---|---|---|---|
| | | | | Yield Relative | |
| | Average Base Count per Device | | | to Untextured | |
| Etch Depth (nm) | Untextured | Inner | Outer | Inner | Outer |
| 304 | 9,421 | 27,716 | 21,490 | 2.9 | 2.3 |
| 426 | 6658 | 24,129 | 17,563 | 3.6 | 2.6 |

Cross Talk. Cross talk occurs when occurs when polynucleotides from an adjacent cluster are inadvertently extracted. On average, cross talk for inner clusters occurred at a rate of 0.8%, whereas cross talk for outer clusters occurred at a rate of 1.8%, showing a 1.0% improvement for the inner-cluster arrangement.

### Example 3

### Patterning of a Wet Deposited Organo-Silicon Containing Molecule

A silicon dioxide wafer was treated with a single organic layer deposited at different locations on the wafer to create loci with a high surface energy and coupling ability to nucleoside. A surface of 1000 Angstroms of silicon dioxide on top of polished silicon was selected. A controlled surface density of hydroxyl groups was achieved on the surface by a wet process using a 1% solution of N-(3-triethoxysilylpropyl)-4-hydroxybutyramidein ethanol and acetic acid deposited on the surface and treated for 4 hours, followed by placing the wafers on a hot plate at 150 degrees C. for 14 hours.

A layer of MEGAPOSIT SPR 3612 photoresist was deposited on top of the N-(3-triethoxysilylpropyl)-4-hydroxybutyramide. In this case, the organic layer was an adhesion promoter for the photoresist. The photoresist layer was patterned by exposure to ultraviolet light through a shadow mask. The photoresist pattern was transferred into the organic layer by oxygen plasma. The photoresist was then stripped, revealing a pattern of regions for biomolecular coupling. Clusters of 80 discs with a diameter of about 80 μm were well resolved.

Polynucleotides were extended from the surface. The photolithographic process performed without adhesion promoter layer did not result in organized loci having polynucleotides extended (data not shown). Polynucleotides extension performed a surface treated with the photolithographic process performed using the N-(3-triethoxysilylpropyl)-4-hydroxybutyramide layer resulted in clarified small discs of polynucleotides 80 μm in diameter located within a cluster of discs.

### Example 4

### Synthesis of a 50-Mer Sequence on a Polynucleotide Synthesis Device

A polynucleotide synthesis device was assembled into a flowcell, which was connected to an Applied Biosystems (ABI394 DNA Synthesizer). The polynucleotide synthesis device was uniformly functionalized with N-(3-triethoxysilylpropyl)-4-hydroxybutyramide (Gelest, CAS No. 156214-80-1) and was used to synthesize an exemplary oligonucleotide of 50 bp (“50-mer oligonucleotide”) using oligonucleotide synthesis methods described herein. The sequence of the 50-mer was as described in SEQ ID NO.: 1.

5′AGACAATCAACCATTTGGGGTGGACAGCCTTGACCTCTAGACTTCGGCAT# #TTTTTTTTTT3′ (SEQ ID NO.: 1), where # denotes Thymidine-succinyl hexamide CED phosphoramidite (CLP-2244 from ChemGenes), which is a cleavable linker enabling the release of oligos from the surface during deprotection. The synthesis was done using standard DNA synthesis chemistry (coupling, capping, oxidation, and deblocking) according to the protocol in Table 4 and an ABI synthesizer.

TABLE 4

| General DNA Synthesis | Table 4 | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

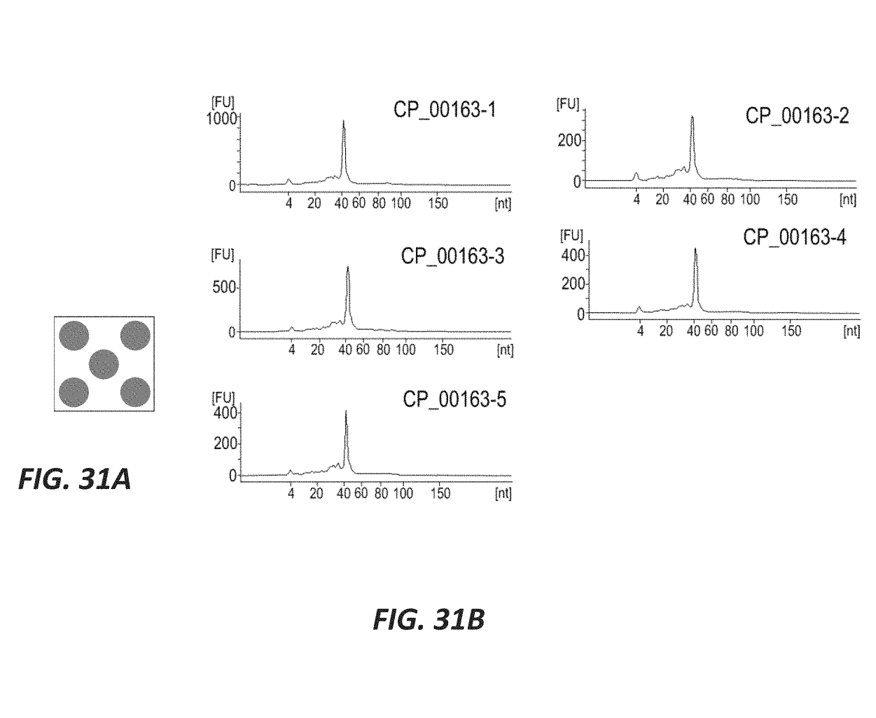

The phosphoramidite/activator combination was delivered similar to the delivery of bulk reagents through the flowcell. No drying steps were performed, as the environment stays “wet” with reagent the entire time. The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I2 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times were adjusted accordingly, and an extra ACN wash was introduced between different chemicals. After oligonucleotide synthesis, the chip was deprotected in gaseous ammonia overnight at 75 psi. Five drops of water were applied to the surface to recover polynucleotides (FIG. 31A). The recovered polynucleotides were then analyzed on a BioAnalyzer small RNA chip (FIG. 31B).

### Example 5

### Synthesis of a 100-Mer Sequence on an Polynucleotide Synthesis Device

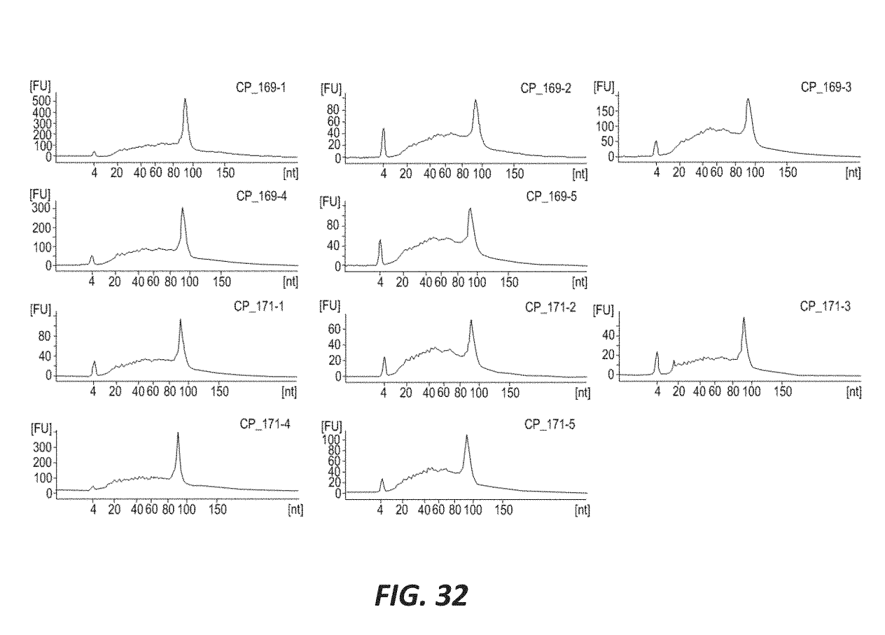

The same process as described in Example 4 for the synthesis of the 50-mer sequence was used for the synthesis of a 100-mer oligonucleotide (“100-mer oligonucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT##TTTTTTTTTT3′, where # denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the oligos extracted from the surface were analyzed on a BioAnalyzer instrument (FIG. 32).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 mastermix, 2.5 uL 10 uM Forward primer, 2.5 uL 10 uM Reverse primer, 1 uL oligo extracted from the surface, and water up to 50 uL) using the following thermalcycling program:

98 C, 30 sec

98 C, 10 sec; 63 C, 10 sec; 72 C, 10 sec; repeat 12 cycles

72 C, 2 min

The PCR products were also run on a BioAnalyzer (data not shown), demonstrating sharp peaks at the 100-mer position. Next, the PCR amplified samples were cloned, and Sanger sequenced. Table 5 summarizes the results from the Sanger sequencing for samples taken from spots 1-5 from chip 1 and for samples taken from spots 6-10 from chip 2.

TABLE 5

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized oligonucleotides were repeated on two chips with different surface chemistries. Overall, 89%, corresponding to 233 out of 262 of the 100-mers that were sequenced were perfect sequences with no errors.

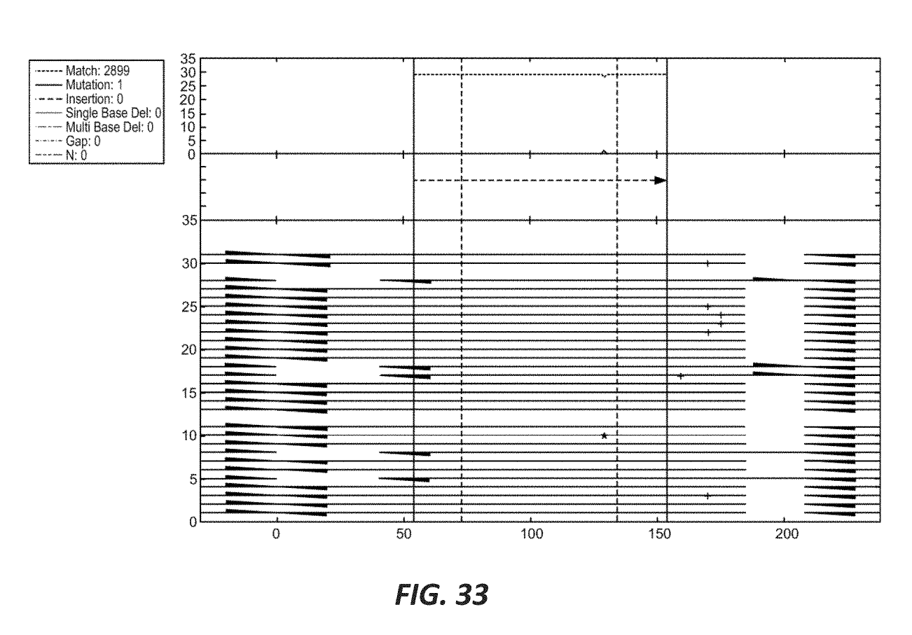

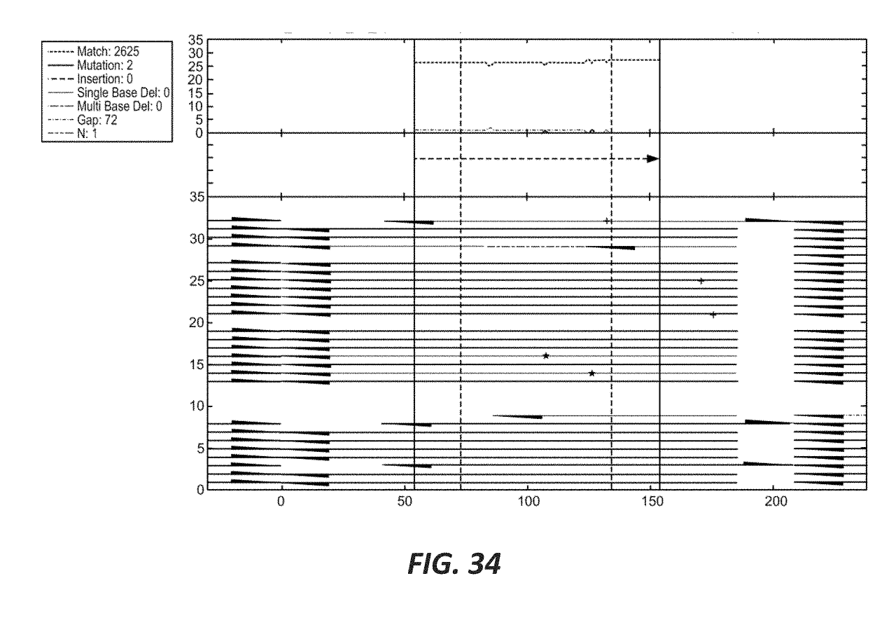

FIGS. 28 and 29 show alignment maps for samples taken from spots 8 and 7, respectively, where “×” denotes a single base deletion, “star” denotes single base mutation, and “+” denotes low quality spots in Sanger sequencing. The aligned sequences in FIG. 33 together represent an error rate of about 97%, where 28 out of 29 reads correspond to perfect sequences. The aligned sequences in FIG. 34 together represent an error rate of about 81%, where 22 out of 27 reads correspond to perfect sequences. Finally, Table 6 summarizes error characteristics for the sequences obtained from the oligonucleotides samples from spots 1-10.

TABLE 6

| | Sample ID/Spot no. | | | | |
|---|---|---|---|---|---|
| | OSA_0046/1 | OSA_0047/2 | OSA_0048/3 | OSA_0049/4 | OSA_0050/5 |
| Total Sequences | 32 | 32 | 32 | 32 | 32 |
| Sequencing | 25 of 28 | 27 of 27 | 26 of 30 | 21 of 23 | 25 of 26 |
| Quality | | | | | |
| Oligo Quality | 23 of 25 | 25 of 27 | 22 of 26 | 18 of 21 | 24 of 25 |
| ROI Match | 2500 | 2698 | 2561 | 2122 | 2499 |
| Count | | | | | |
| ROI Mutation | 2 | 2 | 1 | 3 | 1 |
| ROI Multi Base | 0 | 0 | 0 | 0 | 0 |
| Deletion | | | | | |
| ROI Small | 1 | 0 | 0 | 0 | 0 |
| Insertion | | | | | |
| ROI Single Base | 0 | 0 | 0 | 0 | 0 |
| Deletion | | | | | |
| Large Deletion | 0 | 0 | 1 | 0 | 0 |
| Count | | | | | |
| Mutation: G > A | 2 | 2 | 1 | 2 | 1 |
| Mutation: T > C | 0 | 0 | 0 | 1 | 0 |
| ROI Error Count | 3 | 2 | 2 | 3 | 1 |
| ROI Error Rate | Err: ~1 in | Err: ~1 in | Err: ~1 in | Err: ~1 in | Err: ~1 in |
| | 834 | 1350 | 1282 | 708 | 2500 |
| ROI Minus | MP Err: ~1 | MP Err: ~1 | MP Err: ~1 | MP Err: ~1 | MP Err: ~1 |
| Primer Error | in 763 | in 824 | in 780 | in 429 | in 1525 |
| Rate | | | | | |
| | Sample ID/Spot no. | | | | |
| | OSA_0051/6 | OSA_0052/7 | OSA_0053/8 | OSA_0054/9 | OSA_0055/10 |
| Total Sequences | 32 | 32 | 32 | 32 | 32 |
| Sequencing | 29 of 30 | 27 of 31 | 29 of 31 | 28 of 29 | 25 of 28 |
| Quality | | | | | |
| Oligo Quality | 25 of 29 | 22 of 27 | 28 of 29 | 26 of 28 | 20 of 25 |
| ROI Match | 2666 | 2625 | 2899 | 2798 | 2348 |
| Count | | | | | |
| ROI Mutation | 0 | 2 | 1 | 2 | 1 |
| ROI Multi Base | 0 | 0 | 0 | 0 | 0 |
| Deletion | | | | | |
| ROI Small | 0 | 0 | 0 | 0 | 0 |
| Insertion | | | | | |
| ROI Single Base | 0 | 0 | 0 | 0 | 0 |
| Deletion | | | | | |
| Large Deletion | 1 | 1 | 0 | 0 | 0 |
| Count | | | | | |
| Mutation: G > A | 0 | 2 | 1 | 2 | 1 |
| Mutation: T > C | 0 | 0 | 0 | 0 | 0 |
| ROI Error Count | 1 | 3 | 1 | 2 | 1 |
| ROI Error Rate | Err: ~1 in | Err: ~1 in | Err: ~1 in | Err: ~1 in | Err: ~1 in |
| | 2667 | 876 | 2900 | 1400 | 2349 |
| ROI Minus | MP Err: ~1 | MP Err: ~1 | MP Err: ~1 | MP Err: ~1 | MP Err: ~1 in |
| Primer Error | in 1615 | in 531 | in 1769 | in 854 | 1451 |
| Rate | | | | | |

### Example 6

### Nanoreactor

A nanoreactor was sealed to a silicon wafer. The wafer contained nucleic acids generated from the DNA synthesis reaction. Gene assembly reagents were added to the reaction chamber. Gene amplification occurred in the resolved enclosure. The reaction chamber included nucleic acids encoding for different predetermined sequences. A series of enzymatic reactions resulted in the linking of amplified nucleic acids into a 2 kilobase gene.

### Example 9

### Error Correction of Assembled Nucleic Acids

A gene of about 1 kb (SEQ ID NO.: 5; Table 7) was assembled using 6 purchased oligonucleotides (5 nM each during PCA) (Ultramer; SEQ ID NO.: 6-11; Table 7) and assembled in a PCA reaction using a 1× NEB Q5 buffer with 0.02 U/uL Q5 hot-start high-fidelity polymerase and 100 uM dNTP as follows:

1 cycle: 98 C, 30 sec

15 cycles: 98 C, 7 sec; 62 C 30 sec; 72 C, 30 sec

1 cycle: 72 C, 5 min

TABLE 7

| Nucleic Acid | Sequence |
|---|---|
| Assembled Gene, | 5′ATGACCATGATTACGGATTCACTGGCCGTCGTTTTACAACGTCGTGACTGG |
| SEQ ID NO.: 5 | GAAAACCCTGGCGTTACCCAACTTAATCGCCTTGCAGCACATCCCCCTTTCG |
| | CCAGCTGGCGTAATAGCGAAGAGGCCCGCACCGATCGCCCTTCCCAACAGT |
| | TGCGCAGCCTGAATGGCGAATGGCGCTTTGCCTGGTTTCCGGCACCAGAAG |
| | CGGTGCCGGAAAGCTGGCTGGAGTGCGATCTTCCTGAGGCCGATACTGTCG |
| | TCGTCCCCTCAAACTGGCAGATGCACGGTTACGATGCGCCCATCTACACCAA |
| | CGTGACCTATCCCATTACGGTCAATCCGCCGTTTGTTCCCACGGAGAATCCG |
| | ACGGGTTGTTACTCGCTCACATTTAATGTTGATGAAAGCTGGCTACAGGAAG |
| | GCCAGACGCGAATTATTTTTGATGGCGTTAACTCGGCGTTTCATCTGTGGTG |
| | CAACGGGCGCTGGGTCGGTTACGGCCAGGACAGTCGTTTGCCGTCTGAATTT |
| | GACCTGAGCGCATTTTTACGCGCCGGAGAAAACCGCCTCGCGGTGATGGTG |
| | CTGCGCTGGAGTGACGGCAGTTATCTGGAAGATCAGGATATGTGGCGGATG |
| | AGCGGCATTTTCCGTGACGTCTCGTTGCTGCATAAACCGACTACACAAATCA |
| | GCGATTTCCATGTTGCCACTCGCTTTAATGATGATTTCAGCCGCGCTGTACT |
| | GGAGGCTGAAGTTCAGATGTGCGGCGAGTTGCGTGACTACCTACGGGTAAC |
| | AGTTTCTTTATGGCAGGGTGAAACGCAGGTCGCCAGCGGCACCGCGCCTTTC |
| | GGCGGTGAAATTATCGATGAGCGTGGTGGTTATGCCGATCGCGTCACACTA |
| | CGTCTGAACGTCGAAAACCCGAAACTGTGGAGCGCCGAAATCCCGAATCTC |
| | TATC3′ |
| Assembly | 5′ATGACCATGATTACGGATTCACTGGCCGTCGTTTTACAACGTCGTGACTGG |
| Oligonucleotide | GAAAACCCTGGCGTTACCCAACTTAATCGCCTTGCAGCACATCCCCCTTTCG |
| 1, SEQ ID NO.: 6 | CCAGCTGGCGTAATAGCGAAGAGGCCCGCACCGATCGCCCTTCCCAACAGT |
| | TGCGCAGCC 3′ |
| Assembly | 5′GATAGGTCACGTTGGTGTAGATGGGCGCATCGTAACCGTGCATCTGCCAG |
| Oligonucleotide | TTTGAGGGGACGACGACAGTATCGGCCTCAGGAAGATCGCACTCCAGCCAG |
| 2, SEQ ID NO.: 7 | CTTTCCGGCACCGCTTCTGGTGCCGGAAACCAGGCAAAGCGCCATTCGCCAT |
| | TCAGGCTGCGCAACTGTTGGGA3′ |
| Assembly | 5′CCCATCTACACCAACGTGACCTATCCCATTACGGTCAATCCGCCGTTTGTT |
| Oligonucleotide | CCCACGGAGAATCCGACGGGTTGTTACTCGCTCACATTTAATGTTGATGAAA |
| 3, SEQ ID NO.: 8 | GCTGGCTACAGGAAGGCCAGACGCGAATTATTTTTGATGGCGTTAACTCGG |
| | CGTTTCATCTGTGGTGCAACGG3′ |
| Assembly | 5′GCCGCTCATCCGCCACATATCCTGATCTTCCAGATAACTGCCGTCACTCCAG |
| Oligonucleotide | CGCAGCACCATCACCGCGAGGCGGTTTTCTCCGGCGCGTAAAAATGCGCTC |
| 4, SEQ ID NO.: 9 | AGGTCAAATTCAGACGGCAAACGACTGTCCTGGCCGTAACCGACCCAGCGC |
| | CCGTTGCACCACAGATGAAACG 3′ |
| Assembly | 5′AGGATATGTGGCGGATGAGCGGCATTTTCCGTGACGTCTCGTTGCTGCAT |
| Oligonucleotide | AAACCGACTACACAAATCAGCGATTTCCATGTTGCCACTCGCTTTAATGATG |
| 5, SEQ ID NO.: | ATTTCAGCCGCGCTGTACTGGAGGCTGAAGTTCAGATGTGCGGCGAGTTGC |
| 10 | GTGACTACCTACGGGTAACAGTTT 3′ |
| Assembly | 5′GATAGAGATTCGGGATTTCGGCGCTCCACAGTTTCGGGTTTTCGACGTTCA |
| Oligonucleotide | GACGTAGTGTGACGCGATCGGCATAACCACCACGCTCATCGATAATTTCAC |
| 6, SEQ ID NO.: | CGCCGAAAGGCGCGGTGCCGCTGGCGACCTGCGTTTCACCCTGCCATAAAG |
| 11 | AAACTGTTACCCGTAGGTAGTCACG 3′ |

Ultramer oligonucleotides are expected to have error rates of at least 1 in 500 nucleotides, more likely at least 1 in 200 nucleotides or more.

The assembled gene was amplified in a PCR reaction using a forward primer (5′ ATGACCATGATTACGGATTCACTGGCC3′ SEQ ID NO.: 12) and a reverse primer (5′GATAGAGATTCGGGATTTCGGCGCTCC3′ SEQ ID NO.: 13), using 1× NEB Q5 buffer with 0.02 U/uL Q5 hot-start high-fidelity polymerase, 200 uM dNTP, and 0.5 uM primers as follows:

1 cycle: 98 C, 30 sec

30 cycles: 98 C, 7 sec; 65 C 30 sec; 72 C, 45 sec

1 cycle: 72 C, 5 min

The amplified assembled gene was analyzed in a BioAnalyzer and cloned. Mini-preps from ˜24 colonies were Sanger sequenced. The BioAnalyzer analysis provided a broad peak and a tail for the uncorrected gene, indicated a high error rate. The sequencing indicated an error rate of 1/789 (data not shown). Two rounds of error correction were followed using CorrectASE (Life Technologies, www.lifetechnologies.com/order/catalog/product/A14972) according to the manufacturer's instructions. The resulting gene samples were similarly analyzed in the BioAnalyzer after round one and round two and cloned. (Data not shown.) 24 colonies were picked for sequencing. The sequencing results indicated an error rate of 1/5190 bp and 1/6315 bp after the first and second rounds of error correction, respectively.

### Example 7

### Polynucleotide Distribution for Synthesizing Genes Under 1.8 Kb in Length

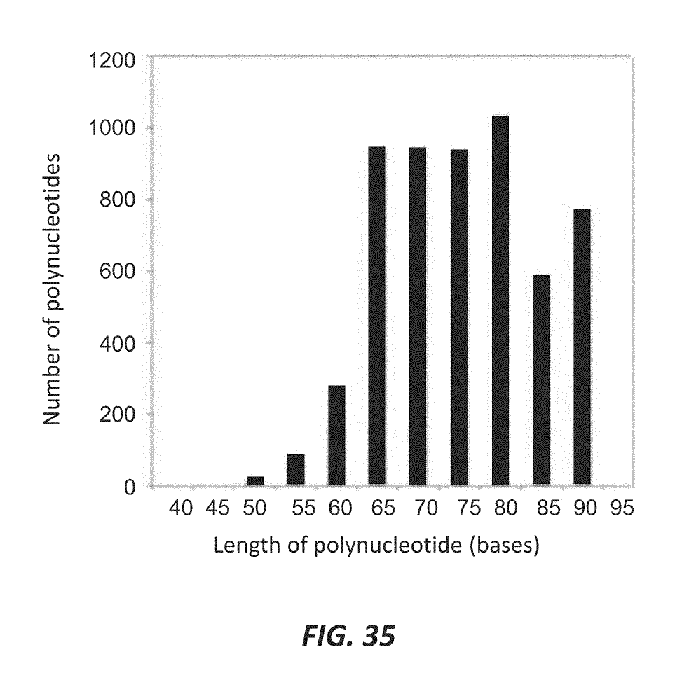

240 genes were selected for de novo synthesis wherein the genes ranged from 701 to 1796 base pairs in length. The gene sequence for each of the genes was divided into smaller fragments encoding for polynucleotides ranging between 50 to 90 polynucleotides in length, with each nucleotide having 20 to 25 nucleotides overlapping sequences. A distribution chart is depicted in FIG. 35, where the X axis depicts the polynucleotide length, and the Y axis depicted the number of polynucleotides synthesized. A total of roughly 5,500 polynucleotides were synthesized on silicon containing molecule coated surface using a protocol similar to that of Example 3, and assembled using a polymerase chain assembly reaction to anneal overlapping sequence of each oligonucleotide to a different oligonucleotide to form a gene.

### Example 8

### Polynucleotide Distribution for Synthesizing a Long Gene Sequence

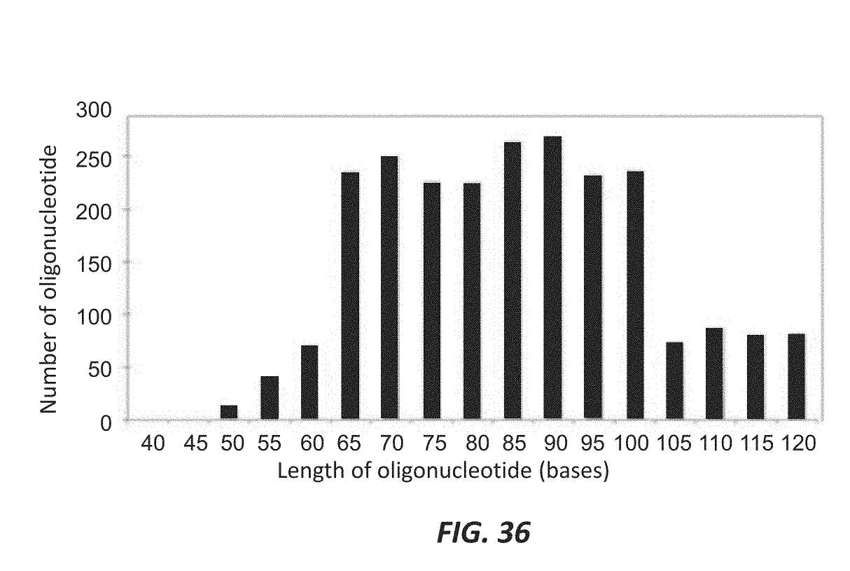

Gene sequence for a single gene that was larger than 1.8 kb in length was divided into smaller fragments encoding for polynucleotides ranging between 50 to 120 nucleotides in length, with each nucleotide having 20 to 25 nucleotides overlapping sequences. A total of 90 different design arrangements were synthesized. A distribution chart is depicted in FIG. 36, where the X axis depicts the polynucleotide length, and the Y axis depicted the number of polynucleotides synthesized. The polynucleotides were synthesized on an organo-silicon containing molecule coated surface using a protocol similar to that of Example 4, and assembled using a polymerase chain assembly reaction to anneal overlapping sequence of each oligonucleotide to a different oligonucleotide to form a gene.

### Example 9

### Two-Step Deposition Process for Dilution of Nucleoside Coupling Agent

Various methods for surface preparation were preformed, which include: (i) performing an active chemical vapor (CVD) deposition step before photolithography; (ii) performing an active chemical vapor (CVD) deposition step after photolithography; and (i) performing a dilution active chemical vapor (CVD) deposition step after photolithograph.

A first silicon device having a silicon oxide layer was individually cleaned in an oxygen plasma (referred to as the “HAPS” chip. An organo-silicon containing molecule, N-(3-TRIETHOXYSILYLPROPYL)-4-HYDROXYBUTYRAMIDE, HAPS), was deposited on the silicon oxide at predetermined locations, referred to as loci. The surface was coated with AZ resist and then baked. The surface was cleaned again in an oxygen plasma, fluorinated (depositing (tridecafluoro-1,1,2,2-tetrahydrooctyl)-trichlorosilane), and then stripped.

A second silicon device having a silicon oxide layer was individually cleaned in an oxygen plasma (referred to as the “100% GOPS-1” chip). An organo-silicon containing molecule, 3-glycidoxypropyltrimethoxysilane (GOPS), was deposited on the silicon oxide at predetermined locations, referred to as loci. The surface was coated with AZ resist and then baked. The surface was cleaned again in an oxygen plasma, fluorinated (depositing (tridecafluoro-1,1,2,2-tetrahydrooctyl)-trichlorosilane), and then stripped.

A third silicon device having a silicon oxide layer was individually cleaned in an oxygen plasma (referred to as the “100% GOPS-2” chip). Directly after cleaning, the surface was coated with AZ resist and then baked at 90 degrees Celsius for 7 min. The surface was cleaned again in an oxygen plasma, fluorinated in the YES CVD system (depositing (tridecafluoro-1,1,2,2-tetrahydrooctyl)-trichlorosilane), and then stripped. The surface was treated with 100% GOPS at 1 Torr for 1 hour at chamber temperature of 100 degrees Celsius. Lastly, the surface was activated in water for 30 minutes at room temperature.

For the diluted active agent deposition protocol, a fourth silicon device having a silicon oxide layer was individually cleaned in an oxygen plasma (referred to as the “GOPS-diluted” chip). Directly after cleaning, the surface was coated with AZ resist and then baked at 90 degrees Celsius for 7 min. The surface was cleaned again in an oxygen plasma, fluorinated in the YES CVD system (depositing (tridecafluoro-1,1,2,2-tetrahydrooctyl)-trichlorosilane), and then stripped. The surface was treated with 100% propyltrimethoxysilane at 3.5 Torr for 15 hours at chamber temperature of 100 C degrees Celsius. The surface was treated with water for 30 minutes, followed by a second deposition step. In the second deposition step, a mixture of 0.05% GOPS and 99.95% propyltrimethoxysilane was deposited on the surface and treated at 3.5 Torr for 1.5 hours at chamber temperature of 100 C. Lastly, the surface was activated in water for 30 minute at room temperature. For each device prepared, the molecules able to couple nucleoside (HAPS and GOPS) were deposited in described locations on the surfaces, loci, and the loci were arranged in clusters.

### Example 10

### Characterization of Low Density Polynucleotide Surfaces

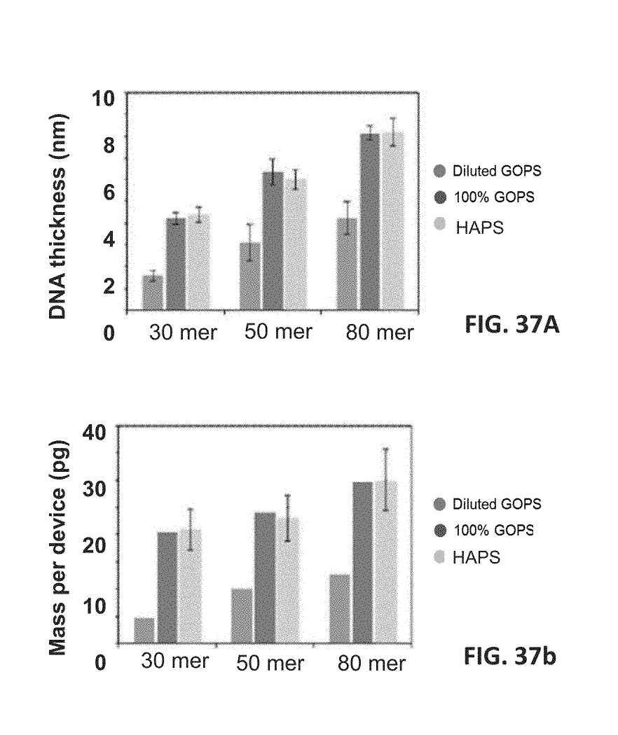

Polynucleotides of 30, 50, and 80 nucleotides in length were synthesized on the surfaces prepared in Example 9. A FRT tool (MicroProf 100, Fries Research and Technology, GmbH, Germany) was used to measure the thickness of DNA post-synthesis. The FRT tool scans across a cluster and measure reflectivity of light, which corresponds to the amount of material on the surface. A summary of the results is shown in FIG. 37A for growth of 30, 50 and 80-mers. In the case of synthesizing 80-mers, the GOPS-diluted surfaces produced a DNA thickness 52% lower than the 100% GOPS-2 and the HAPS chips.

Qubit analysis from fluorometric measurement of the clusters was also performed. A summary of the analysis from growth of 30, 50 and 80-mers is in the chart in FIG. 37B. For 30, 50 and 80-mers, the GOPS-diluted chip resulted in 42% less DNA density than the100% GOPS-2 or the HAPS chips.

### Example 11

### Deletion Error Rate Analysis for Low Density Polynucleotide Surfaces

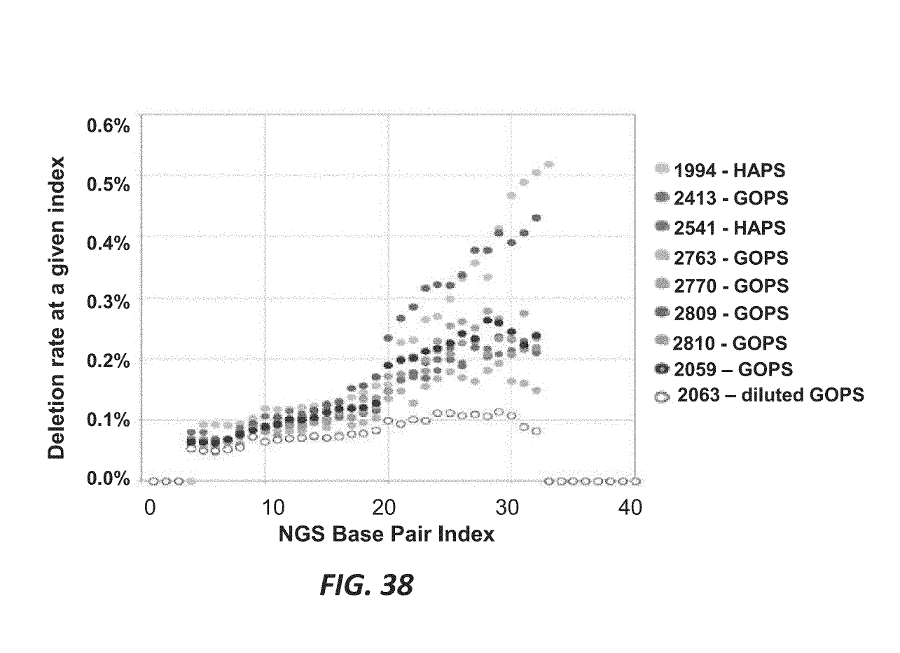

Polynucleotides of 30, 50 and 80 nucleotides in length were synthesized on the surfaces prepared in Example 9, gas cleaved from the surface, and subject to sequence analysis using an Illumina MiSeq. Deletion error rates were determined for polynucleotides synthesized on the GOPS-diluted surfaces. The total deletion error rate was 0.060%, or 1 in 1674 bases. Sequencing of control polynucleotides from the GOPS-diluted chips resulted in a deletion rate of 0.070%. Analysis of the deletion error rate frequency at particular bases in terms of distance from the surface was performed, and results are shown in the plot in FIG. 38. Notably, the GOPS-diluted surfaces resulted in a deletion error rate frequency for bases closer to the surface, which is less than twice the error rate frequency for bases further from the surface. In other words, compared to other surfaces analyzed, the GOPS-diluted surfaces reduce the increase in deletion error rate observed at bases closer to the surface. As a whole, the GOPS-diluted surfaces resulted in lower average deletion error rate above background error rate levels, Table 8.

TABLE 8

| | Active agent | Average deletion |
|---|---|---|
| | added before | error rate above |
| Surface | or after photoresist | background levels |
| GOPS-diluted (no. 2063) | After | 0.03% |
| GOPS-100%-2 (no. 2059) | After | 0.09% |
| GOPS-100%-1 (no. 2413) | Before | 0.07% |
| GOPS-100%-1 (no. 2763) | Before | 0.06% |
| GOPS-100%-1 (no. 2770) | Before | 0.09% |
| GOPS-100%-1 (no. 2809) | Before | 0.15% |
| GOPS-100%-1 (no. 2810) | Before | 0.08% |
| HAPS (no. 1994) | Before | 0.14% |
| HAPS (no. 2541) | Before | 0.07% |

### Example 12

### Performance of Devices Comprising Textured Surfaces

A microfluidic device was manufactured to have increased surface area. An array of posts was etched into a silicon dioxide wafer to increase surface area by a factor of 2 to 3. A number of steps were performed to make a textured surface. To the starting silicon dioxide wafer, with one side polished, was added a textured layer via a plasma enhanced process (PECVD). A passive coating using perfluorooctyltrichlorosilane was used. A silicon reactive ion etching and resist strip was added to the chip, followed by oxidation of the surface. The thermal oxide layer serves as an etch mask for the silicon. The fiducial layer was printed on via lithography, after which a final oxide etching results in a textured silicon ship. The surface had a post width that is about 2 times the length of the desired polynucleotides to be extended.

A chart of exemplary width is provided in Table 9 based on an approximate length of 0.34 nm/base.

TABLE 9

| No. of bases | Oligo length (nm) | Width of post or recess (nm) |
|---|---|---|
| 1 | 0.34 | 0.68 |
| 10 | 3.4 | 6.8 |
| 100 | 34 | 68 |
| 200 | 68 | 136 |
| 300 | 102 | 204 |

A silicon device was prepared having a 16×16 array of clusters, each cluster having 121 loci, the device having 30,976 loci in total. Each cluster includes multiple groups of 4 loci, wherein each loci resides on top of a different design feature, as outlined in Table 10.

TABLE 10

| Design | Width w (nm) | Pitch p (nm) | Depth d (nm) |
|---|---|---|---|
| D1 | 200 | 400 | 250-1000 |
| D2 | 300 | 600 | 250-1000 |
| D3 | 400 | 800 | 250-1000 |
| D4 | No texture | No texture | No texture |

Each locus was coated with glycidoxypropyltriethoxysilane (GOPS) that binds the surface and couples to nucleoside phosphoramidite. Polynucleotide synthesis was performed on the device and DNA thickness, DNA mass and error rates of the synthesized polynucleotides were measured.

### Example 13

### Error Rate by Texture Design

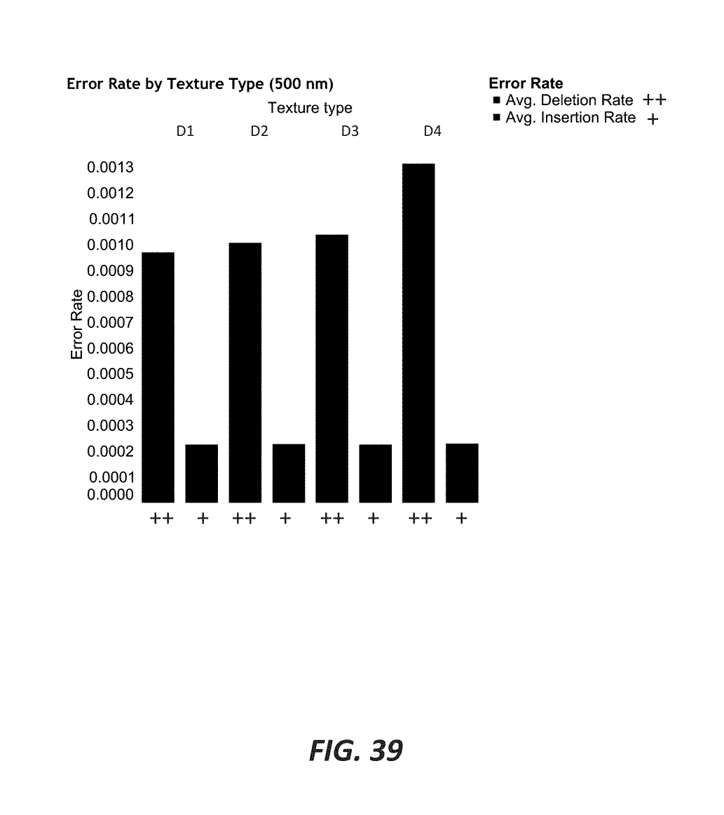

Microfluidic devices were manufactured to have increased surface area according to the designs D1, D2, and D3 of Table 10 outlined in Example 12 at an etch depth of 500 nm. FIG. 39 illustrates that the average deletion rate of the textured designs D1, D2, and D3 were approximately 1/1000, whereas the average deletion rate of the untextured design of D4 was 1.3/1000. The average deletion rate of the textured designs D1, D2, and D3 were lower than that of the untextured D4. The average insertion rate is approximately the same for textured design D1, D2, and D3, and untextured design D4 at 2/1000.

### Example 14

### Yield Enhancement by Texture Design

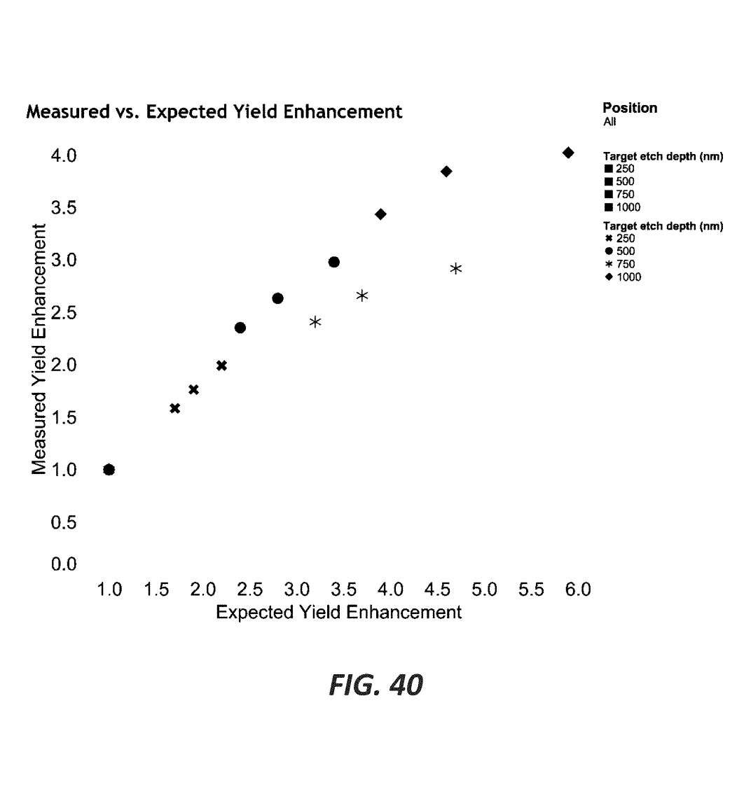

Microfluidic devices were manufactured to have a textured surface with dimensions according to the designs D1, D2, and D3 of Table 10 outlined in Example 12 at etch depths of 250 nm, 500 nm, 750 nm, and 1000 nm. Polynucleotides 92 bases in length were synthesized under each condition. FIG. 40 shows the measured yield and the amount of DNA that was extracted per device, for the textured surfaces normalized to flat surfaces. The yield enhancements of the textured surfaces at an etch depth of 250 nm was approximately 1.5, the yield was approximately 2-3 at etch depths of 500 nm and 750, and the yield was approximately 3-4 at an etch depth of 1000 nm. The expected yield enhancements were calculated based on surface area calculations. As shown in FIG. 40, the measured yield enhancements of the textured designs scaled with the surface area calculations of expected yield enhancements.

### Example 15

### Deletion Rate by Oligonucleotide Base

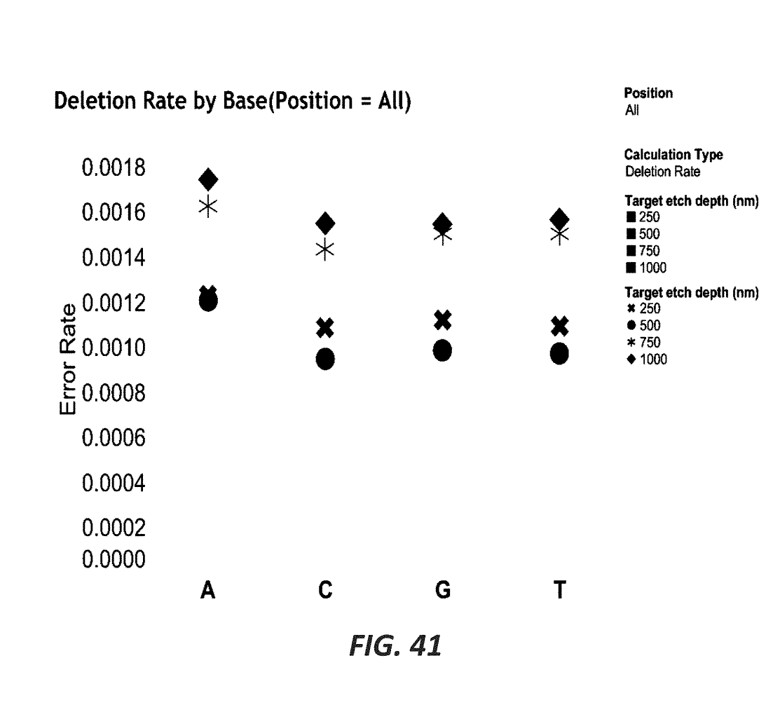

Microfluidic devices were manufactured to have a textured surface with dimensions according to the designs D1, D2, and D3 of Table 10 outlined in Example 12 at etch depths of 250 nm, 500 nm, 750 nm, and 1000 nm Polynucleotides 92 bases in length were synthesized on the different surface types. FIG. 41 illustrates different deletion rates of different bases of the oligonucleotides, depending on the etching depth of the recesses in the microfluidic device. For bases A, C, G, and T, etch depths of 1000 nm provided oligonucleotides with a deletion rate of 14/10,000 to 18/10,000. An etch depth of 750 nm provided oligonucleotides with a slightly lower deletion rate at 14/10,000 to 17/10,000. Etch depths of 500 nm and 250 nm provided oligonucleotides with a deletion rate of 10/10,000 (or 1/1000) to 12/10,000. Overall, etch depths of 250 nm and 500 nm resulted lower deletion rates than at etch depths of 750 nm and 1000 nm.

### Example 16

### Deletion Rate by Texture Condition or Etch Depth

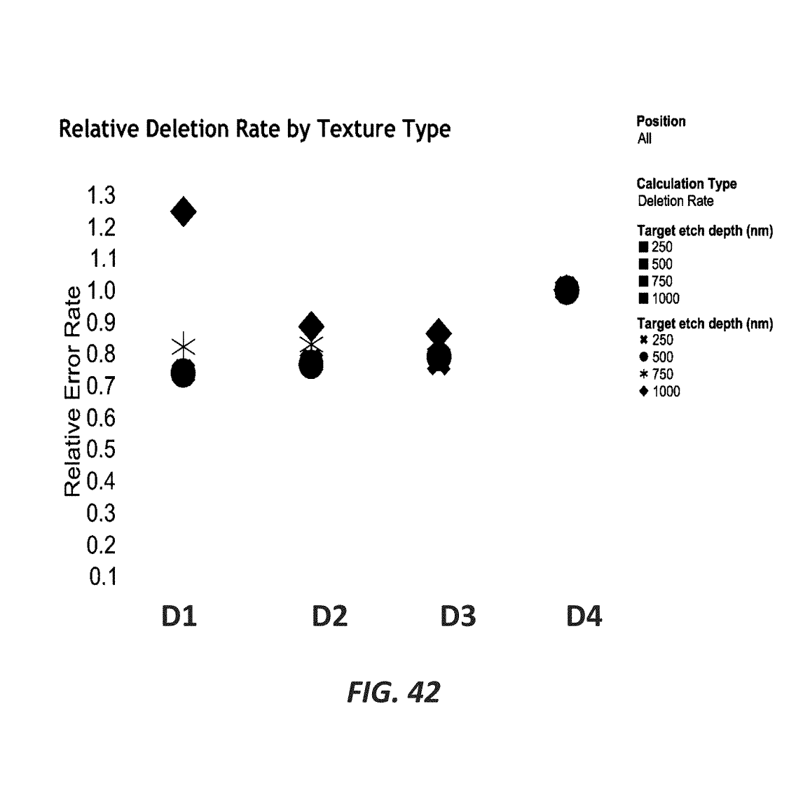

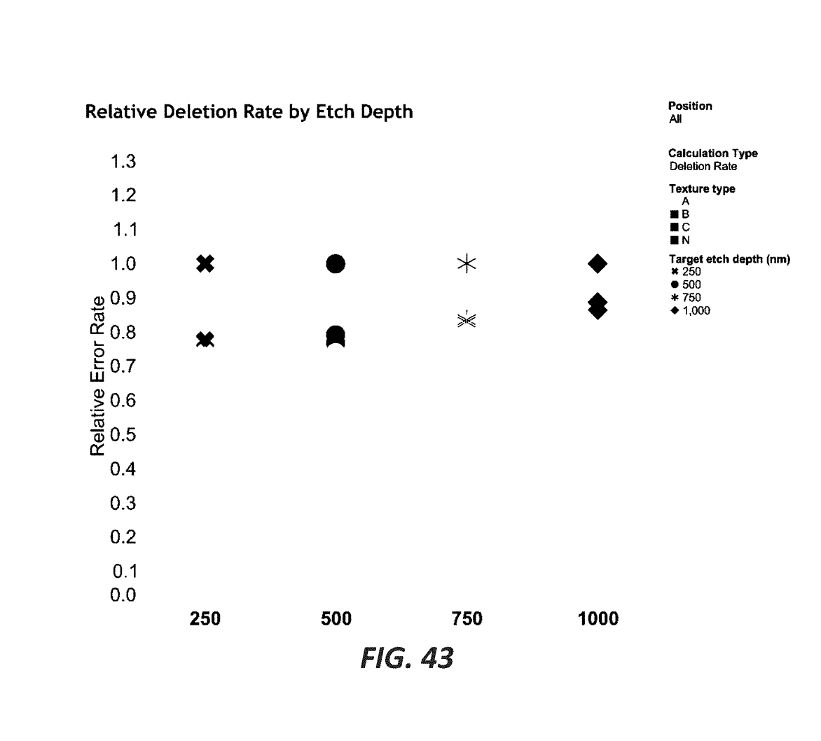

Microfluidic devices were manufactured to have a textured surface with dimensions according to the designs D1, D2, and D3 of Table 10 outlined in Example 12 at etch depths of 250 nm, 500 nm, 750 nm, and 1000 nm. Polynucleotides 92 bases in length were synthesized on the different surface types. FIG. 42 and FIG. 43 illustrate different relative deletion rates of the texture conditions when compared to D4, the untextured design.

### Example 17

### Insertion Rate by Base or Texture Condition

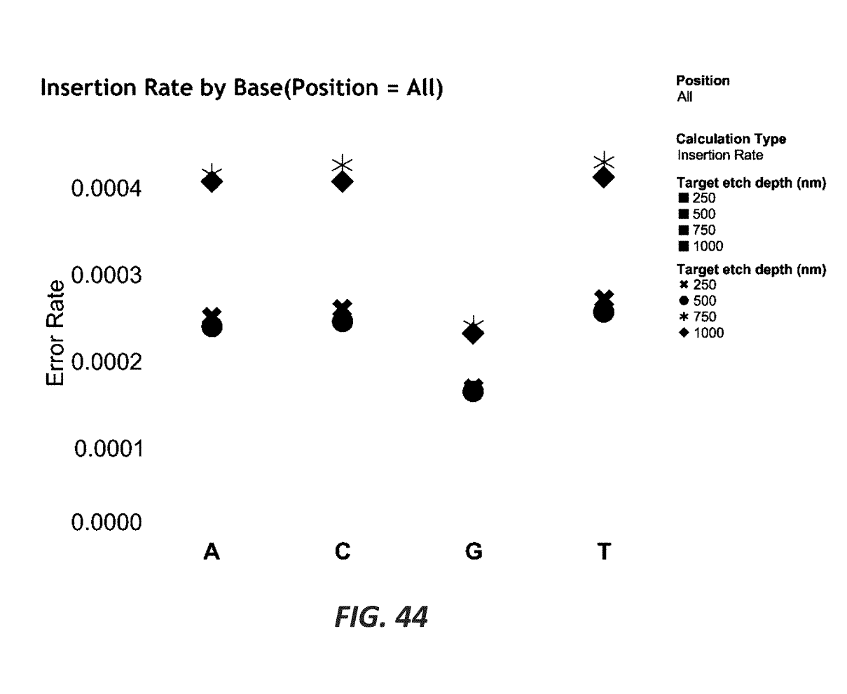

Microfluidic devices were manufactured to have a textured surface with dimensions according to the designs D1, D2, and D3 of Table 9 outlined in Example 12 at etch depths of 250 nm, 500 nm, 750 nm, and 1000 nm. Polynucleotides 92 bases in length were synthesized on the different surface types. FIG. 44 illustrates different insertion rates of different bases of the oligonucleotides, depending on the etching depth of the recesses in the microfluidic device. For bases A, C, and T, etch depths of 750 nm and 1000 nm provided oligonucleotides with insertion rates of approximately 4/1000, and etch depths 250 nm and 500 nm provided oligonucleotides with insertion rates of approximately 2/1000. For base G, etch depths of 750 nm and 1000 nm provided oligonucleotides with an insertion rate of approximately 2/1000, and etch depths 250 nm and 500 nm provided oligonucleotides with an insertion rate of approximately 1.5/1000.

### Example 18

### Relative Insertion Rate by Texture Condition

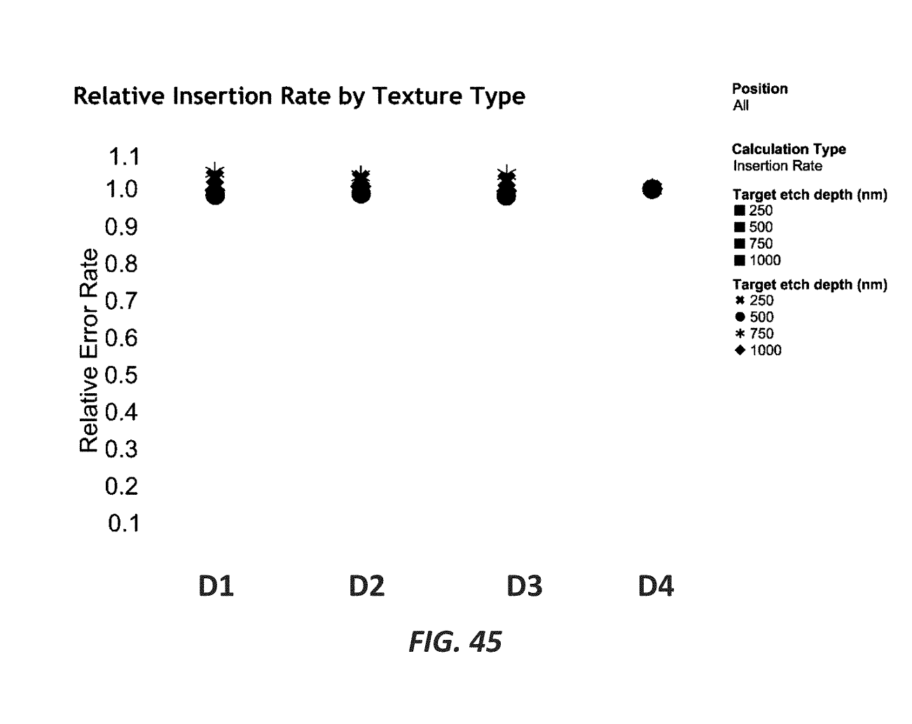

Microfluidic devices were manufactured to have a textured surface with dimensions according to the designs D1, D2, and D3 of Table 10 outlined in Example 12 at etch depths of 250 nm, 500 nm, 750 nm, and 1000 nm. Polynucleotides 92 bases in length were synthesized on the different surface types. FIG. 45 illustrates different relative insertion rates of the texture conditions when compared to D4, the untextured design. The insertion rates of the textured surfaces of D1, D2, and D3 were similar to the insertion rate of the untextured surface, with relative insertion rates about 1.0.

### Example 19

### Array of 256 Clusters

A microfluidic device was manufactured. Each device was a 200 mm wafer, double-side polished. A SOI wafer had 21 chips arranged in a 200 mm wafer. Each chip is 32 mm×32 mm in size, and comprised a 16×16 array of clusters. A total of 256 clusters were present in the array. 121 reaction sites are located in a single cluster, providing 30,976 individually addressable oligo sites per chip. Each cluster pitch was 1.125mm. Each of the reaction sites are about 50 μm in diameter.

### Example 20

### Fiducial Marks

A silicon dioxide is prepared having a 16×16 array of clusters. Each cluster includes groups of 4 loci, wherein each locus is in close proximity to a one of three fiducial marks having a plurality of lines, wherein the line weight is listed in Table 11. Each fiducial design is in the shape of a plus 805. One of the regions includes a plurality of fiducial marks in close proximity 810.

TABLE 11

| Design | Width w (um) | Pitch p (um) |
|---|---|---|
| 1 | 0.2 | 0.4 |
| 2 | 0.3 | 0.6 |
| 3 | 0.4 | 0.8 |

Each locus is coated with an organo-silicon containing molecule that binds the surface and couples to nucleoside phosphoramidite (e.g., HAPS or GOPS). Polynucleotide synthesis is perform on the device and measurements are taken using the fiducial marks to calibrate align the surface with other components of a system.

### Example 21

### Global Alignment Marks

A silicon device is prepared having a 16×16 array of clusters. Global alignment marks are used to aligning the surface 1000 with other components of a system. Global alignment marks 1005, 1010, 1015, 1020, 1035, and 1040 are located at positions on a substantially planar substrate portion of the surface 1000 and near an edge of the device. Detailed circular mark 1025 and plus sign mark 1045 are shown in an expanded view in FIG. 10.

### Example 22

### Coating a Textured Surface with Diluted Activating Agent

A device for polynucleotide synthesis is manufactured. Each device is 200 mm, double-side polished. A SOI wafer has 21 chips are arranged in a 200 mm wafer. Each chip is 32 mm×32 mm in size, and comprised a 16×16 array of clusters. A total of 256 clusters are present in the array. 121 reaction sites are located in a single cluster, providing 30,976 individually addressable oligo sites per chip. Each cluster pitch is 1.125 mm. Each of the reaction sites are about 50 μm in diameter. The surface of each chip is textured by methods as described in Example 12 to include recesses with one of the texture designs listed in Table 1.

The devices for polynucleotide synthesis are individually cleaned by treatment with oxygen plasma. Directly after cleaning, the surface is coated with AZ resist and then baked at 90 degrees Celsius for 7 minutes. The surface is cleaned again by treatment with oxygen plasma, fluorinated in a YES CVD system (depositing (tridecafluoro-1,1,2,2-tetrahydrooctyl)-trichlorosilane), and is stripped. The surface is treated with 100% propyltrimethoxysilane at 3.5 Torr for 15 hours at chamber temperature of 100 degrees Celsius. The surface is treated with water for 30 minutes, followed by a second deposition step. In the second deposition step, a mixture of 0.05% GOPS and 99.95% propyltrimethoxysilane is deposited on the surface and treated at 3.5 Torr for 1.5 hours at chamber temperature of 100 degrees Celsius. Lastly, the surface is activated in water for 30 minute at room temperature. The reaction sites on the surface of each device comprise diluted GOPS. Each of the reaction sites are surrounded by surface coated with tridecafluoro-1,1,2,2-tetrahydrooctyl)-trichlorosilane.

### Example 23

### Alternative Oligonucleotide Synthesis Protocol

A similar protocol is followed according to Example 9, wherein the protocol involves additional acetonitrile washes. An extra acetonitrile wash flow is performed directly before the deblocking process. An extra acetonitrile wash flow is also performed directly after the deblocking process.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A device for polynucleotide synthesis, the device comprising:
a solid support comprising a surface;
a plurality of loci on the surface, wherein each of the loci comprises:
an inner region, wherein the inner region comprises a plurality of recesses or protrusions; and
an outer region that comprises a plurality of first molecules, wherein the outer region spans and extends beyond the inner region, wherein each of the first molecules binds to the surface and comprises a reactive group capable of binding to a nucleoside, and wherein the outer region of each of the loci is non-overlapping with the outer region for another of the loci.

2. The device of claim 1, wherein the plurality of loci are arranged in clusters.

3. The device of claim 2, wherein each cluster comprises 50 to 500 loci.

4. The device of claim 1, wherein the outer region has a diameter of up to 100 um.

5. The device of claim 1, wherein the inner region has a diameter of about 55 um.

6. The device of claim 1, wherein the inner region has a diameter 80% to 95% shorter than a diameter of the outer region.

7. The device of claim 1, wherein the inner region has a diameter 2 um to 20 um shorter than a diameter of the outer region.

8. The device of claim 1, wherein each of the recesses or protrusions has an etch depth of 100 um to 1000 nm.

9. The device of claim 1, wherein each of the recesses or protrusions has a width of 100 um to 500 um.

10. The device of claim 1, wherein each of the recesses or protrusions has a pitch length of about 2 to 3 times a width of the recesses or protrusions.

11. The device of claim 1, wherein each of the recesses or protrusions has a depth of about 60% to 125% of a pitch length.

12. The device of claim 1, wherein each of the recesses or protrusions has a pitch of up to 1 um.

13. The device of claim 1, wherein the solid support has a tensile strength of 1 MPa to 300 MPa.

14. The device of claim 1, wherein the solid support has a tensile strength of 1 MPa to 10 MPa.

15. The device of claim 1, wherein the solid support has a stiffness of 1 GPa to 500 GPa.

16. The device of claim 1, wherein the solid support has a stiffness of 1 GPa to 10 GPa.

17. The device of claim 1, wherein the solid support comprises nylon, nitrocellulose, or polypropylene.

18. The device of claim 1, wherein the solid support comprises silicon, silicon dioxide, silicon nitride, polytetrafluoroethylene, polypropylene, polystyrene, polycarbonate, gold, or platinum.

19. The device of claim 1, wherein each of the first molecules is a silane.

20. The device of claim 19, wherein the silane is an aminosilane.

21. The device of claim 20, wherein each of the first molecules is N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane, 3-iodo-propyltrimethoxysilane, or octylchlorosilane.

22. The device of claim 1, further comprising a plurality of second molecules, wherein the plurality of second molecules is located on the surface in a region surrounding the outer region of each of the loci, and wherein each of the second molecules binds to the surface and lacks a reactive group capable of binding to the nucleoside.

23. The device of claim 22, wherein each of the second molecules is a fluorosilane.

24. The device of claim 23, wherein the fluorosilane is (tridecafluoro-1,1,2,2-tetrahydrooctyl)trichlorosilane, perfluorooctyltrichlorosilane, perfluorooctyltriethoxysilane, or perfluorooctyltrimethoxychlorosilane.

25. A method for polynucleotide synthesis, comprising:
(a) providing predetermined sequences for polynucleotides;
(b) providing the device of claim 1; and
(c) synthesizing the polynucleotides.

26. A method for gene synthesis, comprising:
(a) providing predetermined sequences for polynucleotides;
(b) providing the device of claim 1;
(c) synthesizing the polynucleotides; and
(d) assembling the polynucleotides to form a plurality of genes.


SEQUENCE LISTING (13 sequences)

SEQ ID NO: 1 - DNA, 62 bp, Artificial Sequence
  Description of Artificial Sequence Synthetic oligonucleotide
  modified_base (51)..(52): Thymidine-succinyl hexamide CED phosphoramidite
agacaatcaaccatttggggtggacagccttgacctctagacttcggcattttttttttttt

SEQ ID NO: 2 - DNA, 112 bp, Artificial Sequence
  Description of Artificial Sequence Synthetic polynucleotide
  modified_base (101)..(102): Thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, Artificial Sequence
  Description of Artificial Sequence Synthetic primer
atgcggggttctcatcatc

SEQ ID NO: 4 - DNA, 20 bp, Artificial Sequence
  Description of Artificial Sequence Synthetic primer
cgggatccttatcgtcatcg

SEQ ID NO: 5 - DNA, 931 bp, Artificial Sequence
  Description of Artificial Sequence Synthetic polynucleotide
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatc

SEQ ID NO: 6 - DNA, 163 bp, Artificial Sequence
  Description of Artificial Sequence Synthetic polynucleotide
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcc

SEQ ID NO: 7 - DNA, 175 bp, Artificial Sequence
  Description of Artificial Sequence Synthetic polynucleotide
gataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttggga

SEQ ID NO: 8 - DNA, 176 bp, Artificial Sequence
  Description of Artificial Sequence Synthetic polynucleotide
cccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgg

SEQ ID NO: 9 - DNA, 176 bp, Artificial Sequence
  Description of Artificial Sequence Synthetic polynucleotide
gccgctcatccgccacatatcctgatcttccagataactgccgtcactccagcgcagcaccatcaccgcgaggcggttttctccggcgcgtaaaaatgcgctcaggtcaaattcagacggcaaacgactgtcctggccgtaaccgacccagcgcccgttgcaccacagatgaaacg

SEQ ID NO: 10 - DNA, 177 bp, Artificial Sequence
  Description of Artificial Sequence Synthetic polynucleotide
aggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagttt

SEQ ID NO: 11 - DNA, 178 bp, Artificial Sequence
  Description of Artificial Sequence Synthetic polynucleotide
gatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacg

SEQ ID NO: 12 - DNA, 27 bp, Artificial Sequence
  Description of Artificial Sequence Synthetic primer
atgaccatgattacggattcactggcc

SEQ ID NO: 13 - DNA, 27 bp, Artificial Sequence
  Description of Artificial Sequence Synthetic primer
gatagagattcgggatttcggcgctcc